SEQUENCE LISTING (10 sequences)

SEQ ID NO: 1 - DNA, 20 bp, Artificial Sequence
  Primer
tttgcccgagaaagactagc

SEQ ID NO: 2 - DNA, 20 bp, Artificial Sequence
  Primer
catttggcccttcgattaga

SEQ ID NO: 3 - DNA, 19 bp, Artificial Sequence
  Primer
tgcgcaagctttccaagat

SEQ ID NO: 4 - DNA, 20 bp, Artificial Sequence
  Primer
cagcgagttggtgagcatga

SEQ ID NO: 5 - DNA, 24 bp, Artificial Sequence
  Primer
gacaactggtggcagatttcgctt

SEQ ID NO: 6 - DNA, 24 bp, Artificial Sequence
  Primer
agccacaaagaaaggagttggacc

SEQ ID NO: 7 - DNA, 21 bp, Artificial Sequence
  Primer
atctgcaccggcccagctact

SEQ ID NO: 8 - DNA, 21 bp, Artificial Sequence
  Primer
ccaccgcgaaggccccaaata

SEQ ID NO: 9 - DNA, 20 bp, Artificial Sequence
  Primer
aaacaccggagcggacaggc

SEQ ID NO: 10 - DNA, 20 bp, Artificial Sequence
  Primer
ggtcgcggtcagacgtggtg

(19) United States
(12) Patent Application Publication
(10) Pub. No.: US 2022/0251504 A1
(43) Pub. Date: Aug. 11, 2022

(54) FUNCTIONAL ASTROCYTES DERIVED FROM PLURIPOTENT STEM CELLS AND METHODS OF MAKING AND USING THE SAME

(71) Applicant: New York Stem Cell Foundation, Inc., New York, NY (US)

(72) Inventors: Valentina Fossati, New York, NY (US); Matthew Zimmer, New York, NY (US); Lilianne Barbar, New York, NY (US); Tanya Jain, New York, NY (US)

(21) Appl. No.: 17/614,916

(22) PCT Filed: May 29, 2020
(86) PCT No.: PCT/US2020/035391

§ 371 Date: Nov. 29, 2021

Related U.S. Application Data
(60) Provisional application No. 62/912,497, filed on Oct. 8, 2019.
(60) Provisional application No. 62/864,750, filed on Jun. 21, 2019.
(60) Provisional application No. 62/854,207, filed on May 29, 2019.

(51) Int. Cl. C12N 5/079; A61K 35/30

(52) U.S. Cl. CPC C12N 5/0622; A61K 35/30; C12N 2501/58; C12N 2501/12; C12N 2501/135; C12N 2501/105

(57) ABSTRACT

Described is the efficient and robust generation and isolation of functional astrocytes from pluripotent stem cells (PSCs). The methodology provided recapitulate the major steps of oligodendrocyte differentiation into mixed cell populations and subsequent isolation of astrocytes from the mixed cell populations.

### CROSS-REFERENCE TO RELATED APPLICATIONS

This application is a U.S. National Stage of International Application No. PCT/US2020/035391, filed May 29, 2020, which claims the benefit of priority of U.S. Provisional Patent Application No. 62/854,207, filed May 29, 2019, U.S. Provisional Patent Application No. 62/864,750, filed Jun. 21, 2019, and U.S. Provisional Patent Application No. 62/912,497, filed Oct. 8, 2019, the contents of each of which are hereby incorporated by reference in their entireties.

### STATEMENT AS TO RIGHTS TO INVENTIONS MADE UNDER FEDERALLY SPONSORED RESEARCH AND DEVELOPMENT

This invention was made with government support under 1R21 NS11186-01 awarded by the National Institute of Neurological Disorders and Stroke (NINDS) of the National Institute of Health (NIH). The government has certain rights in the invention.

### INCORPORATION OF SEQUENCE LISTING

The material in the accompanying sequence listing is hereby incorporated by reference into this application. The accompanying sequence listing text file, named NYS_015WOUS_SEQ_L.txt, was created on Nov. 22, 2021, and is 2,322 bytes. The file can be accessed using Microsoft Word on a computer that uses Windows OS.

### BACKGROUND OF THE INVENTION

### Field of the Invention

The present invention relates generally to cell culture, and more particularly to a composition and method for generating and isolating CD49f-positive astrocytes.

### Background Information

Astrocytes play a critical role in the central nervous system (CNS) by maintaining brain homeostasis, providing metabolic support to neurons, regulating connectivity of neural circuits, and controlling blood flow as an integral part of the blood-brain barrier. They also undergo a pronounced transformation called reactive astrogliosis following injury and in disease. Accumulating evidence has implicated astrocytes, such as A1 reactive astrocytes, in the onset and progression of many neurological diseases, prompting increased efforts to identify novel astrocyte targets for therapeutic intervention.

Studies of astrocyte biology have often relied on reductionist cell culture models, of which many have been produced since the 1970s. The first method to purify primary astrocytes from rodent brains was based on selective adhesion to tissue culture plates, which eliminates most non-astrocytes, but still retains contaminating cells—largely microglia and oligodendrocyte lineage cells. Furthermore, astrocytes selected with this method are primarily immature, proliferating cells, and require serum to grow in vitro, which induces a reactive pathological state. While these methods have been extremely powerful for understanding important astrocyte functions, due to inducing a baseline pathological state, they have had limited success in investigating disease states of astrocytes.

More recently, immunopanning methods that take advantage of the cell surface antigens Integrin Beta-5 (encoded by Itgb5) to purify rodent astrocytes and GlialCAM (or HepaCAM) adhesion protein to purify human astrocytes (in rodents, HepaCAM is also highly expressed by oligodendrocyte progenitor cells) have become more commonly used. Commercial magnetic-activated cell sorting based on GLAST (Slc1a3) and ASCA-2 has also been widely adopted. All these methods allow isolation of post-mitotic astrocytes, and in the case of immunopanning, allow maintaining cells in serum-free conditions. Nonetheless, these methods have been largely focused on rodent cells, whereas access to human primary CNS cells has been largely limited by the availability of brain specimens. Therefore, knowledge of astrocyte biology has been mainly built from rodent models, either in vivo or in vitro. Recently, mounting evidence has revealed important astrocyte contributions to the development and progression of neurological diseases, such as Alzheimer's disease (AD), Parkinson's disease (PD) and progressive multiple sclerosis (MS), for which optimal astrocyte-specific animal models are lacking. Moreover, it is now clear that human astrocytes have several distinct features from their rodent counterparts, which could well be driving pathogenic mechanisms of human diseases. Taken together, there is an urgent need for better human in vitro modeling of astrocyte (dys)function.

Human induced pluripotent stem cell (hiPSC) technology has emerged as a powerful tool to generate human astrocytes and other CNS cells in vitro, starting from skin fibroblasts or peripheral blood mononuclear cells of patients and healthy individuals. Protocols to differentiate hiPSCs into astrocytes use either a specific gradient of patterning agents to mimic embryonic development or overexpression of transcription factors. Many of these protocols require a few consecutive passages to eliminate neuronal cells and achieve a mature state. Alternative 3D cultures of CNS organoids generate neural progenitor cells, neurons, oligodendrocyte lineage cells, astrocytes, and can incorporate microglia. With organoids being increasingly utilized to model CNS diseases, methods for purifying specific cell types are becoming highly desirable for downstream analyses. The GlialCAM marker used for purifying adult primary astrocytes via immunopanning is not expressed in hiPSC-derived astrocytes until 100 days in culture, prioritizing the need for a novel method to isolate astrocytes at earlier time points.

Thus, because of the important role of astrocytes in the CNS, there is a need for new methods of isolating astrocytes to investigate astrocyte function.

### SUMMARY OF THE INVENTION

Provided herein, are methods of generating, isolating and purifying astrocytes, such as those differentiated from stem cells and hiPSCs. Methods of purifying astrocytes include use of astrocyte cell-surface marker CD49f that is expressed in fetal and adult brains from healthy and diseased individuals.

Also provided herein are single-cell and bulk transcriptome analyses of CD49f⁺ hiPSC-astrocytes. Isolated CD49f⁺ hiPSC-astrocytes can perform key astrocytic functions in vitro, including trophic support of neurons, glutamate uptake, and phagocytosis. Notably, CD49f⁺ hiPSC-astrocytes respond to inflammatory stimuli, acquiring an A1-like reactive state, in which they display impaired phagocytosis and glutamate uptake and fail to support neuronal maturation. Importantly, conditioned medium from human reactive A1-like astrocytes is toxic to human and rodent neurons. CD49f⁺ hiPSC-astrocytes provided herein are thus a valuable resource for investigating human astrocyte function and dysfunction in health and disease.

In an embodiment, the invention provides a method for isolating an astrocyte from a mixed population of cells. The method includes: a) selecting for a CD49f+ cell from the mixed population; and b) sorting and isolating the CD49f+ cell from the mixed population, wherein the CD49f+ cell is a CD49f+ astrocyte, thereby isolating the astrocyte.

In another embodiment, the invention provides a method of generating and isolating an astrocyte. The method includes: a) generating a mixed population of cells by culturing a stem cell (SC) under conditions to induce neuronal differentiation; b) selecting for a CD49f+ cell from the mixed population of cells; and c) isolating the CD49f+ cell from the mixed population of cells, wherein the CD49f+ is a CD49f+ astrocyte, thereby generating and isolating the astrocyte.

In yet another embodiment, the invention provides a kit. The kit includes: a) an antibody that selectively binds CD49f; and b) one or more reagents for generating, culturing and/or isolating a CD49f+ astrocyte.

In still another embodiment, the invention provides a method of co-culturing an astrocyte isolated via a method of the invention with a neuronal cell. In various aspects, co-culturing generates an organoid.

In another embodiment, the invention provides a method of treating a neurological disease or disorder in a subject. The method includes administering to the subject an effective amount of a CD49f+ astrocyte isolated via a method of the invention, or an organoid produced by co-culturing an isolated astrocyte with a neuronal cell, thereby treating the neurological disease or disorder in the subject.

In yet another embodiment, the invention provides a non-human mammal including an astrocyte isolated via a method of the invention.

### BRIEF DESCRIPTION OF THE FIGURES

FIG. 1 shows a timeline of oligodendrocyte differentiation following Option A (FIG. 1A) and Option B (FIG. 1B). Triangles represent the recommended time points to evaluate the expression of stage-specific markers through immunofluorescence. SB: SB431542; LDN: LDN193189; SAG: Smoothened Agonist; T3: triiodothryonine; RA: all trans retinoic acid; PDGF: platelet derived growth factor; HGF: hepatocyte growth factor; IGF-I: insulin like growth factor-1; NT3: neurotrophin 3; AA: ascorbic acid.

FIG. 2 shows the requirement for RA and SHH to derive OLIG2⁺ progenitor cells. FIG. 2A shows live imaging and flow cytometric quantification of OLIG2-GFP cells at day 14 of differentiation under different conditions for RA and SHH. RA at a concentration of 100 nM from day 0 results in the highest yield of GFP⁺ cells. FIG. 2B shows a comparison between the addition of SHH or SAG at day 8 to the best RA condition, via live imaging and fluorescent activated cell sorting (FACS)-analysis. Negative: human embryonic stem cell (hESC) line RUES1. FIG. 2C shows temporal gene expression profile of PAX6, OLIG2 and NKY2.2 under optimal RA and SHH conditions. Error bars are SEM (N=3). Comparable results are obtained using RUES1 cells and the OLIG2-GFP reporter line. FIG. 2D shows an assessment of sphere-formation for unsorted cells, sorted GFP⁺ cells, or GFP-cells. Only GFP⁺ cells form spheres, providing enrichment to the GFP⁺ population.

FIG. 3 shows SHH and dual SMAD inhibition requirements for OLIG2⁺ progenitors. FIG. 3A shows mRNA levels for OLIG2, PTCH1 and SHH at day 14 of differentiation between cultures with RA from d0 and SHH/SAG from d8, compared to cells that were exposed only to RA from d0. Endogenous SHH levels are higher in the samples that were not exposed to SHH/SAG. Error bars are SEM (N=3). FIG. 3B shows FACS-analysis for O4⁺ cells at day 75 of differentiation between cells that were exposed to RA only, or RA and SAG, in the first 12 days of differentiation. A 56% decrease in the O4⁺ population was observed in the cells that were not provided with SAG in the initial steps of differentiation. Negative Control: APC-conjugated secondary antibody only. FIG. 3C shows live imaging and FACS-analysis at day 14 of differentiation under different SMAD inhibitors. A substantial population of GFP⁺ cells is present only under the dual inhibition of SMAD proteins, indicating a synergistic effect between RA and the dual inhibition of SMAD. DSi: Dual SMAD inhibition; SB: SB431542; LDN: LDN189193.

FIG. 4 shows generation of oligodendrocytes from human pluripotent stem cells. FIG. 4A shows a diagram of the differentiation protocol from human pluripotent stem cells (hPSCs) to mature oligodendrocytes. FIG. 4B-4M are sequential steps of in vitro oligodendrocyte differentiation of RUES1 cells showing: PAX6⁺ neural stem cells at day 8 (B), phase contrast of the multilayered structures at day 12 (C), OLIG2⁺NKX2.2⁺ pre-OPC at day 18 (D), SOX10⁺OLIG2⁺ early OPCs (E), live imaging of O4⁺ late OPCs (F), cropped image of O4⁺ cells to highlight the ramified processes (G), O4⁺ OPCs co-expressing OLIG2 (H), O4⁺ OPCs co-expressing SOX10 (I), sorted O4⁺ OPCs co-expressing SOX10 and NG2 (J), terminally differentiated MBP⁺ oligodendrocytes at low (K), and higher (×64) magnification (L), MAP2⁺ and GFAP⁺ cells in the oligodendrocyte cultures (M). PSC: pluripotent stem cell; NSC: neural stem cell; OPC: oligodendrocyte progenitor cell; OL: oligodendrocyte; pO/L: poly-L-ornithine/Laminin.

FIG. 5 shows proliferative oligodendrocytes and other neural cell-types. FIG. 5A shows representative immunofluorescence staining at d50 of differentiation for OLIG2, SOX10 and Ki67. There is an almost complete co-localization between OLIG2 and SOX10. FIG. 5B shows temporal quantification of Ki67⁺ cells showing the percentage of OLIG2⁺ and SOX10⁺ progenitors that proliferate between d30 and d50 of differentiation. Error bars are SEM (N=2). FIG. 5C shows an immunofluorescence image of a culture at d73 showing a high number of O4⁺ cells, but hardly any cells co-express Ki67. FIG. 5D shows the percentage of O4⁺ cells that co-express MBP 4 weeks after growth-factor removal (Glial Medium). Error bar is SEM (N=4). FIG. 5E shows the percentage of MAP2⁺ neurons and GFAP⁺ astrocytes to total number of cells in three independent experiments at d78-d88 of differentiation. Error bars are SEM (N=3).

FIG. 6 shows live O4 imaging at day 53 (FIGS. 6A, 6D), day 63 (FIGS. 6B, 6E), and day 73 (FIGS. 6C, 6F) of differentiation following the Option B protocol. FIGS. 6A-6C show representative fields at low magnification; the number of O4⁺ cells increase with time. FIGS. 6D-6F show higher magnification images of FIG. 6A-6C, respectively, to highlight the morphology of the cells. FIG. 6G shows representative examples of O4⁺ cell frequencies calculated by flow cytometry at different time points using the Option B (“fast”) protocol (day 55, day 63 and day 75) and the Option A (“original”) protocol. Scale bars: 500 μm (FIGS. 6A-6C); 200 μm (6D-6F).

FIG. 7 shows the fundamental steps of hPSC differentiation to oligodendrocytes. FIG. 7A shows PAX6⁺ neural stem cells at day 8 of differentiation (PAX6; nuclei are stained with DAPI). FIG. 7B shows typical morphology of the culture at day 12, depicting three-dimensional structures. FIG. 7C shows expression of OLIG2 and NKX2.2 at day 12 as shown through immunofluorescent analysis (OLIG2; NKX2.2; nuclei are stained with DAPI). FIG. 7D shows spheres selection: arrows indicate the good spheres, which are round-shape, golden/brown in color, with darker core and with a diameter between 300 μm and 800 μm. The exclamation mark indicates a pair of joined spheres that can be broken into single spheres by gentle pipetting. Aggregates that should be avoided are small and transparent (arrowheads) or very large and irregular in shape, usually derived by mechanical digestion (star). FIG. 7E shows immunofluorescent staining of progenitor cells at day 56, co-expressing NKX2.2, SOX10 and OLIG2. FIG. 7F shows O4 live staining showing the highly ramified morphology of the cells. FIG. 7G shows MBP⁺ oligodendrocytes at the end of the differentiation (nuclei are stained with DAPI). FIG. 7H shows morphology of MBP⁺ oligodendrocytes at higher magnification (64×). MAP2⁺ and GFAP⁺ cells are also present in the culture. FIG. 7I shows that purified O4⁺ cells 24 hours after sorting still retain the typical ramified morphology. Scale bars: 500 μm (FIG. 7A, 7B, 7G); 200 μm (FIG. 7C, 7E, 7F, 7I); 1 mm (FIG. 7D).

FIG. 8 shows the generation and characterization of primary-progressive multiple sclerosis (PPMS) iPSC lines. FIG. 8A shows mRNA/miRNA reprogramming of PPMS lines, showing representative skin fibroblasts at day 7, iPSC-like colonies evident at day 12, and TRA-1-60⁺ colonies at day 15 of reprogramming. FIG. 8B shows immunofluorescence of PPMS-iPSC 102 for pluripotency markers. FIG. 8C shows immunofluorescence after in vitro spontaneous differentiation through embryoid bodies for endodermal marker AFP, mesodermal marker αSMA and ectodermal marker βIII-tubulin. Nuclei are stained with DAPI. FIG. 8D shows H&E stained sections of in vivo teratoma formation after injection of PPMS iPSCs into immunodeficient mice, showing representative structures from glandular tissue (endoderm), cartilage (mesoderm) and pigmented epithelium (ectoderm). FIG. 8E shows pluripotency gene expression of undifferentiated PPMS-iPSC and parental fibroblasts, relative to a hESC line. ANPEP gene is specific for fibroblasts. FIG. 8F shows cytogenetic analysis; all PPMS-iPSC lines show a normal karyotype.

FIG. 9 shows characterization of teratoma formation in iPSC lines. Representative pictures from endoderm (FIG. 9A), ectoderm (FIG. 9B), and mesoderm (FIG. 9C) of teratoma formation in vivo after transplantation of cells from iPSC line 102 into immunodeficient mouse.

FIG. 10 shows that PPMS-iPSCs generate OPCs and mature oligodendrocytes in vitro. FIG. 10A-10I are sequential steps of in vitro oligodendrocyte differentiation of PPMS iPSC showing: PAX6⁺ cells at day 8 (A), multilayered structures in phase contrast at day 12 (B), OLIG2⁺ and NKX2.2⁺ cells at day 12 (C), SOX10⁺OLIG2⁺ early OPCs (D), live imaging of O4⁺ late OPCs at day 73 (E), cropped image of O4⁺ cells to highlight the ramified processes (F), terminally differentiated MBP⁺ oligodendrocytes in low (G), and higher (×64) magnification (H), MAP2⁺ cells in the oligodendrocyte cultures (I). FIG. 10J shows quantification of O4⁺ cells after 75 days of differentiation from RUES1 and PPMS iPSCs via FACS analysis. Gates are based on secondary Ab-APC only for O4 staining and PE-conjugated isotype control for PDGFRα staining (Negative). Total O4⁺ cells frequency (O4 & ⁺⁺) is shown in parentheses.

FIG. 11 shows characterization of cells before transplantation for the in vivo studies. FIG. 11A shows FACS plots showing pluripotency markers in an iPSC line grown on MEFs, and the lack of the same markers in cells at d81 of differentiation. FIG. 11B is a plot showing the more stringent gate used for sorting O4⁺ cells before isolation and cryopreservation for the in vivo transplantations. FIG. 11C shows a brightfield image of O4⁺ sorted cells plated after cryopreservation, during the recovery period of 48 hours. FIG. 11D shows live O4 staining in sorted OPCs, showing the retention of O4 and the proper ramified morphology.

FIG. 12 shows PPMS-derived OPCs engraft and differentiate to myelinogenic oligodendrocytes in vivo. FIG. 12A shows human PPMS iPSC-derived O4⁺ OPCs transplanted into neonatal shiverer/rag2 mice. At 16 weeks, human cells exhibited dense engraftment in the corpus callosum (hNA). A large number had differentiated as MBP-expressing oligodendrocytes and were distributed diffusely throughout corpus callosum. FIG. 12B is a confocal image showing co-localization of mouse axons (neurofilament; NF) and MBP⁺ human oligodendrocytes. FIG. 12C and FIG. 12D show electron micrographs of myelinated axons exhibiting characteristic compact myelin with alternating major dense (arrowheads) and intraperiod lines. FIG. 12E shows that transplanted human cells retained progenitor characteristics with ˜80% of hNA+ cells expressing OLIG2 (16 weeks). FIG. 12F shows that at 16 weeks, individual NG2 cells had begun to migrate into the overlying cerebral cortex. FIG. 12G shows that iPSC-derived O4-sorted OPCs demonstrated limited bipotentiality in vivo: only a few GFAP⁺ astrocytes were found in proximity to the lateral ventricle.

FIG. 13 shows demographic information of MS patients and healthy individuals from which iPSC lines have been derived.

FIG. 14 shows reprogramming of skin fibroblasts to iPSCs. FIG. 14A shows that fibroblasts with apparent changes in their morphology are visible at d3 of reprogramming. FIG. 14B shows transfection efficiency at d7, evaluated using nGFP mRNA. FIG. 14C shows iPSC-like colonies at d12. FIG. 14D shows an iPSC clone expanded in feeder-free conditions.

FIG. 15 shows characterization of iPSC lines using nanostring analysis for pluripotency. FIG. 15A shows nanostring analysis of 3 hESC lines, 2 fibroblasts lines, and 5 iPSC lines derived from MS patients or healthy controls; iPSC lines are indistinguishable from hESC lines. FIG. 15B shows that spontaneous embryoid body differentiation in vitro gives rise to cells from all germ layers. From left to right: AFP⁺ endodermal cells, Tuji1⁺ ectodermal cells, aSMA⁺ mesodermal cells.

FIG. 16 shows an approach to understanding multiple sclerosis.

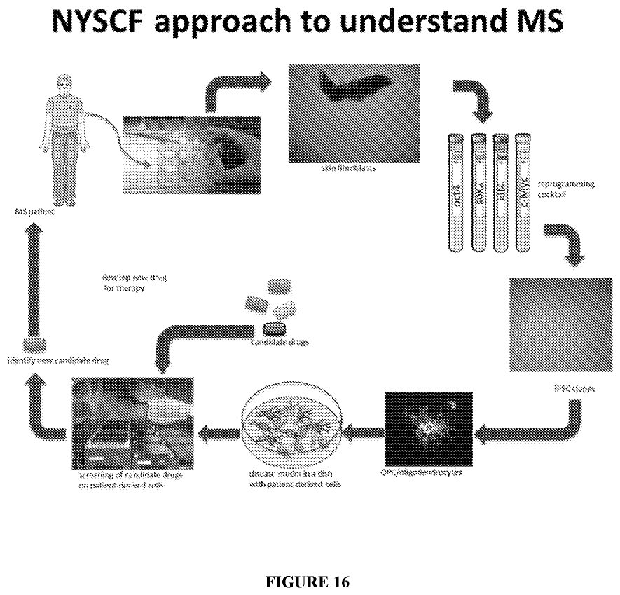

FIG. 17 shows that CD49f surface antigen purifies hiPSC-derived astrocytes. FIG. 17A shows a schematic of a hiPSC-astrocyte differentiation protocol depicting the major steps leading to the CD49f sort. FIG. 17B shows the top hits for astrocyte markers identified from the Lyoplate screen. FIG. 17C shows representative flow-cytometry contour plots (with outliers shown) of the CD49f sort of hiPSC-cells generated from 3 hiPSC lines from three individuals using the astrocyte differentiation protocol in FIG. 17A. FIG. 17D shows a bar chart with individual data points plotted as circles, where dots represent 3 hiPSC lines, displaying the consistent proportion of CD49f+ cells obtained from independent differentiations for each line. Error bars show mean±standard deviation (n=65). At least 5 independent differentiations per line were performed. FIG. 17E shows representative contour plots (with outliers shown) of the EpCAM sort of hiPSC-cells generated from 3 lines. FIG. 17F shows representative immunofluorescence images of CD49f+ and CD49f− cells 24 hours post-sort showing GFAP+ astrocytes, MAP2+ neurons, O4+ immature oligodendrocytes, and total DAPI cells. Scale bar, 100 μm. FIG. 17G shows that a vast majority of sorted CD49f+ cells are also GFAP+, while almost no CD49f− cells are GFAP+ cells. Dots correspond to 3 different lines. Error bars show mean±standard deviation (n=3 independent lines). FIG. 17H shows representative images of individual magnified CD49f+ astrocytes cultured at low density and stained with GFAP, showing their morphological heterogeneity. Each cell was cropped and placed in the image. FIG. 17I shows representative immunofluorescence images of hiPSC-derived CD49f+ astrocytes and primary rat astrocytes stained with GFAP and DAPI, showing that human cells are larger in size. Scale bar, 100 μm. FIG. 17J Bar graph showing the median of the cell area in μm² of GFAP⁺ hiPSC-derived CD49f⁺ astrocytes (n=1885 cells from 3 lines with 1-2 replicates each) is significantly greater than that of primary rat astrocytes (n=1293 cells from 6 replicates). Error bars represent the interquartile range. p-value was calculated using a two-tailed, unpaired t-test.

FIG. 18 shows immunofluorescence and transcription profiling of CD49f+ hiPSC-derived astrocytes, confirming expression of canonical markers (see also FIG. 27). FIG. 18A shows immunofluorescence images of CD49f⁺ astrocytes showing expression of astrocyte markers, and total DAPI cells. Scale bar, 200 μm. FIG. 18B shows immunofluorescence images of CD49f⁺ astrocytes showing CD49f⁺, GFAP⁺, AQP4⁺ and total DAPI cells. Arrows indicate cells that are CD49f⁺/AQP4⁺/GFAP. Scale bar, 50 μm. FIG. 18C shows percentages of CD49f+ cells across different CD49f+ hiPSC-astrocyte lines that are also GFAP+, AQP4+, and triple positive for GFAP, AQP4, and CD49f. Dots correspond to 3 different lines. Error bars show mean±standard deviation (n=5, 1-2 technical replicates per line). FIG. 18D shows RNA-Seq expression levels of immature astrocyte markers in human CD49f⁺ astrocytes, expressed in transcripts per million (TPM). Dots correspond to 3 different lines. Error bars show mean±standard deviation (n=9, 3 replicates per line). FIG. 18E shows mRNA expression levels of mature astrocyte markers in human CD49f⁺ astrocytes, expressed in transcripts per million (TPM). Error bars are as in d). FIG. 18F shows that hierarchical clustering of RNA-Seq data indicates that CD49f⁺ hiPSC-astrocytes (black) closely resemble primary and iPSC-derived astrocytes from independent studies and are distinct from other brain cell types (GEO: GSE73721; GEO: GSE97904. Analysis is based on transcriptome-wide expression. CD49f⁺ samples consist of 3 different lines in 3 replicates each.

FIG. 19 shows single-cell transcriptome data confirming that CD49f⁺ sorting strategy from heterogeneous hiPSC-derived cultures enriches for mature astrocytes. All data from one control line (line 3, n=1). See also FIGS. 28, 29, and 30. FIG. 19A shows tSNE plots of single-cell RNA-Seq data from unsorted (left, n=7,744), CD49f⁺ (middle, n=9,047), and CD49f⁻ sorted (right, n=5,057) cells. In total, 12 clusters were identified. FIG. 19B shows quantification of cell type proportions from unsorted, CD49f⁺, and CD49f⁻ sorted cells based on tSNE analysis. CD49f⁺ sorted cells are mostly astrocytes. FIG. 19C shows tSNE feature plots of CD49f (ITGA6), mature astrocyte (GFAP, AQP4), and immature astrocyte (NUSAP1) transcripts from unsorted, CD49f⁺ sorted, and CD49f⁻ sorted cells, showing that CD49f⁺ cells express primarily mature astrocyte markers. FIG. 19D shows a heatmap of cell type-specific transcript expression across identified clusters. FIG. 19E shows a tSNE plot of all astrocytes from unsorted, CD49f⁺, and CD49f⁻ sorted cells (n=12,061 astrocytes). After subsetting and reintegrating only astrocytes from the initial clustering scheme, 2 immature and 4 mature astrocyte clusters, and 1 astrocyte-like cluster were identified. FIG. 19F shows a heatmap of top differentially expressed genes identified across all astrocyte-related clusters. FIG. 19G shows the top ten differentially expressed genes for each astrocyte-related cluster. Abbreviations: Imm.=immature; Oligo=oligodendrocyte; OPC=oligodendrocyte progenitor cell.

FIG. 20 shows that CD49f-sort from human fetal brain enriches for astrocytes, and that CD49f is localized in astrocytes in slides from human adult brains. See also FIG. 31. FIG. 20A shows representative immunofluorescence images showing vimentin, CD49f, and DAPI in CD49f⁺ and CD49f⁻ sorted cells from human fetal brain tissue. Scale bar, 100 μm. FIG. 20B shows tSNE plots of single-cell RNA-Seq data from unsorted (left, n=11,817), CD49f⁺ (middle, n=6,069), and CD49f⁻ sorted (right, n=4,409) cells from fetal brain tissue. In total, 18 clusters were identified. All data are from an 18-week-old human fetus (n=1). FIG. 20C shows quantification of cell type proportions from unsorted, CD49f⁺, and CD49f⁻ sorted cells from fetal brain tissue. CD49f⁺ cells are highly enriched in astrocytes and immature astrocytes. FIG. 20D shows tSNE feature plots highlighting in cells that express CD49f (ITGA6), mature astrocyte (GFAP), and immature astrocyte (NUSAP1, C3) transcripts from unsorted, CD49f⁺, and CD49f⁻ sorted cells from fetal brain tissue. FIG. 20E shows representative immunofluorescence images showing GFAP⁺, AQP4⁺, and CD49f⁺ cells with DAPI nuclei in cryosections from the subventricular zone of an adult brain from healthy individual. Arrows indicate cells that are CD49f⁺, AQP4⁺, and GFAP⁺. Scale bar, 10 μm. FIG. 20F shows representative immunofluorescence images showing CD49f⁺ and GFAP⁺ cells with DAPI nuclei in cryosections from the prefrontal cortex of an Alzheimer's disease patient. Arrows indicate cells that are CD49f⁺ and GFAP⁺. Arrowheads indicate CD49f⁺ endothelial cells. Scale bar, 10 μm. Abbreviations: Endo.=endothelial cell: Imm.=immature: OPC=oligodendrocyte progenitor cell.

FIG. 21 shows that CD49f⁺ hiPSC-derived astrocytes provide neuronal support and exhibit other astrocytic functions in vitro. See also FIG. 32. FIG. 21A shows representative recordings of firing patterns and spontaneous excitatory post-synaptic currents (sEPSCs) measured in hiPSC-neurons at day 50 when cultured alone, or with CD49f⁺ hiPSC-astrocytes during days 33-50. Neurons co-cultured with astrocytes exhibited more mature firing patterns and a greater number of sEPSCs. FIG. 21B shows that CD49f⁺ hiPSC-astrocytes enhance electrophysiological properties of hiPSC-neurons in co-culture. Bar graphs show the maximum number of evoked spikes per 1 second stimulus (n=8/18), the maximum firing frequency in hertz (Hz) (n=5/15), the amplitude adaptation ratio between first and last action potential (n=5/15), the maximum action potential height (mV) (n=8/17), and the frequency of spontaneous excitatory post-synaptic currents (Hz) (n=5/9) in hiPSC-neurons at day 50 when cultured alone vs. with astrocytes during days 33-50. Dots correspond to 3 different lines. Each dot represents an independent cell. n=neurons alone/neurons co-cultured with astrocytes. Error bars show mean±standard deviation. p-values were calculated using a two-tailed, unpaired t-test. FIG. 21C shows representative immunofluorescence images showing MAP2⁺ neurons, GFAP⁺ astrocytes, and DAPI nuclei in hiPSC-derived neurons at 40 days in vitro cultured alone or with CD49f⁺ hiPSC-astrocytes for one week. Scale bar, 50 μm. FIG. 21D shows the average size of MAP2⁺ cells. Dots correspond to 3 different CD49f⁺ hiPSC-astrocyte lines. Error bars show mean±standard deviation (n=3 technical replicates). p-values were calculated using a two-tailed, unpaired t-test. FIG. 21E shows that CD49f⁺ astrocytes take up glutamate. Bar graphs show percent of glutamate taken up by CD49f⁺ hiPSC-astrocytes after incubation with 100 μM glutamate for 3 hours, compared to wells without cells (media only). Dots correspond to 3 different astrocyte lines. Error bars show mean±standard deviation (n=4 technical replicates). p-values were calculated using a one-way ANOVA with Dunnett's correction for multiple comparisons. FIG. 21F shows shows that hiPSC-astrocytes show spontaneous Ca2⁺ transients. Nine representative traces of spontaneous [Ca²⁺]ᵢ transients from 3 independent CD49f⁺ hiPSC-astrocyte lines loaded with the Ca²⁺ indicator Rhod-3/AM are shown. Each line is represented by a different color (lines 1,2,3). FIG. 21G shows that CD49f⁺ hiPSC-astrocytes respond to ATP. Representative traces of [Ca²⁺]ᵢ transients from nine astrocytes from one iPSC line loaded with the Ca²⁺ indicator Rhod-3/AM following 100 μM ATP application. FIG. 21H shows that CD49f⁺ hiPSC-astrocytes secrete proinflammatory cytokines when stimulated for 24 hours with TNF α, IL-1α, and C1q, or TNF α and IL-1β. Bar charts with individual data points plotted as dots show concentration of cytokines secreted in the supernatant of CD49f⁺ astrocytes with and without stimulation. Concentrations are expressed in pg/ml and normalized to 1,000 cells. Dots correspond to 3 lines (n=6, 2 technical replicates per line). Error bars show mean±standard deviation. p-values were calculated using a one-way ANOVA with Dunnett's correction for multiple comparisons.

FIG. 22 shows that CD49f⁺ hiPSC-derived astrocytes can be stimulated in vitro to take on an A1-like reactive state that loses key astrocytic functions. See also FIG. 33. FIG. 22A shows representative immunofluorescence images showing the reactive marker C3 in CD49f⁺ astrocytes upon stimulation with TNFα, IL-1α, and C1q. Cells are also stained for GFAP and DAPI. White dashed boxes indicate the areas of the magnified images on the right to highlight changes in morphology. Scale bar, 50 μm. FIG. 22B shows the percentage of C3⁺ cells in unstimulated vs. stimulated CD49f⁺ astrocytes as in a). Dots correspond to 3 different lines. Error bars show mean t standard deviation (n=4 independent lines). p-values were calculated using a two-tailed, paired t-test. FIG. 22C shows the cell radial mean, in arbitrary unit (A.U.) across different lines in unstimulated vs. stimulated CD49f⁺ astrocytes, depicting the shift in morphology between the two conditions. Dots correspond to 3 different lines. Error bars show mean±standard deviation (n=3 independent lines). p-values were calculated using a two-tailed, paired t-test. FIG. 22D shows that CD49f⁺ hiPSC-derived astrocytes upregulate the A1 reactive transcripts previously identified in mouse astrocytes. Heat map shows expression levels of reactive transcripts (pan-reactive, A1 astrocytes or A2 astrocytes) in A1-like vs. unstimulated (A0) CD49f⁺ hiPSC-astrocytes. FIG. 22E shows that glutamate uptake is reduced in CD49f⁺ A1-like astrocytes. Percent of glutamate taken up by A0 vs. A1 astrocytes and compared to wells without cells (media only). Error bars show mean±standard deviation (n=2-4 technical replicates). p-values were calculated using multiple t-tests with Holm-Sidak's correction for multiple comparisons. FIG. 22F shows that relative mRNA expression of genes encoding glutamate receptors (GRIN2b, GRIK1, GRIA1), quantified via qPCR analysis in two independent experiments, is decreased in CD49f stimulated astrocytes (A1) vs. unstimulated (A0). Dots correspond to 3 lines. Error bars show mean±standard deviation (n=6, 2 replicates per line). p-values were calculated using a two-tailed, paired t-test. FIG. 22G shows representative images of A0 vs. A1 astrocytes engulfing pHrodo-synaptosomes, showing reduced phagocytic capacity in A1 astrocytes. White dashed boxes indicate the areas of the magnified images on the right. Scale bar, 200 μm. FIG. 22H shows time course analysis and quantification of A0 vs. reactive A1 astrocytes engulfing pHrodo-synaptosomes. The average degree of engulfment (normalized to confluence) with standard error of the mean (n=9-12 replicates per line), indicates a reduced phagocytic capacity in A1 astrocytes. p-values were calculated using a two-way ANOVA. FIG. 22I shows that relative mRNA expression of genes encoding phagocytic receptors (MERTK, MEGF10) and bridging molecule GAS6, quantified via qPCR analysis, is decreased in CD49f⁺ A1 vs. A0. Dots correspond to 3 lines. Error bars show mean±standard deviation (n=6, 2 replicates per line). p-values were calculated using a two-tailed, paired t-test. FIG. 22J shows that human A1-like astrocytes have a stronger ATP response than unstimulated astrocytes. Area under the curve of ΔF/F traces for one minute following 100 μM ATP application of hiPSC-derived CD49f⁺ A0 (n=36 cells) and A1 (n=31 cells) astrocytes loaded with the Ca²⁺ indicator Rhod-3/AM. Unit is in arbitrary fluorescence units (AFUs). Dots correspond to 2 lines. Error bars show mean±standard deviation. p-values were calculated using a two-tailed, unpaired t-test. FIG. 22K shows that relative mRNA expression of ITGA6, quantified via qPCR analysis does not significantly differ between A0 vs. A1 astrocytes in two independent experiments. Dots correspond to 3 different lines. Error bars show mean±standard deviation (n=6, 2 replicates per line). p-values were calculated using a two-tailed, paired t-test. FIG. 22L shows that CD49f protein levels do not significantly differ between A0 vs. A1 astrocytes. Representative western bands and electropherograms for CD49f and b-actin, and quantification of protein levels for CD49f in A0 and A1 astrocytes, normalized to b-actin. Error bars show mean±standard deviation (n=5, 1-2 replicates per line). Dots correspond to 3 different lines. p-values were calculated using a two-tailed, paired t-test.

FIG. 23 shows that A1-like reactive CD49f⁺ hiPSC-derived astrocytes impair neuronal maturation and connectivity and are neurotoxic. See also FIG. 34. FIG. 23A shows a schematic of neuron co-culture experiment with A0 and A1 hiPSC-astrocytes. FIG. 23B shows representative recordings of firing patterns measured in hiPSC-neurons at day 51 when cultured with A0 or A1 astrocytes during days 33-51. Neurons co-cultured with A1 astrocytes exhibited less mature firing patterns. FIG. 23C shows that A1 astrocytes provide markedly lower enhancements of neuronal electrophysiological properties in co-culture than A0 astrocytes. Bar graphs with individual data points plotted show the maximum number of evoked spikes per 1s stimulus (n=28/32), the half-width of the first spike (ms) (n=28/32), the amplitude adaptation ratio between first and last action potential (n=27/22), and the maximum action potential height (mV) (n=26/32) in hiPSC-neurons at day 51 when cultured with A0 or A1 astrocytes during days 33-51. Dots correspond to 3 different lines. Each dot represents an independent cell from which we recorded. n=neurons co-cultured with A0 astrocytes/neurons co-cultured with A1 astrocytes. Error bars show mean±standard deviation. p-values were calculated using a two-tailed, unpaired t-test. FIG. 23D shows representative recordings of spontaneous excitatory post-synaptic currents (sEPSCs) measured in hiPSC-neurons at day 51 when cultured with A0 or A1 astrocytes during days 33-51. Neurons co-cultured with A1 astrocytes exhibited a smaller number of sEPSCs. Representative traces of sEPSCs are each from a different cell. FIG. 23E shows the frequency of spontaneous excitatory post-synaptic currents (Hz) (n=15 co-cultured with A0 astrocytes; 15 co-cultured with A1 astrocytes) in hiPSC-neurons at day 51 when cultured with A0 or A1-like astrocytes during days 33-51. Dots correspond to 3 different lines. Each dot represents an independent cell from which currents were recorded. Error bars show mean±standard deviation. p-values were calculated using a two-tailed, unpaired t-test. FIG. 23F shows that relative mRNA expression of genes encoding synaptogenic factors, quantified via qPCR analysis, is decreased in A1 vs. A0 astrocytes. Dots correspond to 3 different lines. Error bars show mean±standard deviation (n=6, 2 replicates per line). p-values were calculated using a two-tailed, paired t-test. FIG. 23G shows a schematic of A0 vs. A1 astrocyte conditioned media experiment on hiPSC-neurons. FIG. 23H shows representative images of neurons treated with A0 or A1 astrocyte conditioned media (CM) for 3 days, subjected to a caspase 3/7 apoptosis assay and cell nuclei: neurons exposed to A1 CM show increased apoptosis. Scale bar, 100 μm. FIG. 23I shows time course apoptosis analysis and quantification of the percentage of caspase 3/7⁺ neurons during treatment with A0 or A1 astrocyte conditioned media (CM). Error bars represent the standard error of the mean (n=12-24 replicates per line), indicating a neurotoxic effect of A1 CM on neurons. p-values were calculated using a two-way ANOVA. FIG. 23J shows time course apoptosis analysis and quantification of the percentage of caspase 3/7⁺ apoptotic neurons during stimulation with TNFα, IL-1α, and C1q, demonstrating no direct effects of the cytokine cocktail on neuronal apoptosis. Error bars represent the standard error of the mean (n=12 replicates). p-values were calculated using a two-way ANOVA.

FIG. 24 shows that maturation state affects CD49f⁺ hiPSC-derived astrocytes response to stimulation with TNFα, IL1α, and C1q. FIG. 24A shows tSNE plots of single-cell RNA-Seq data from unstimulated (A0, n=5,881) and TNFα, IL-1α, and C1q stimulated (A1, n=6,701) astrocytes, shown by cluster (left) and by treatment type (right). Data from two iPSC lines (lines 1 and 3, n=2). FIG. 24B shows quantification of cell type proportions from unstimulated (A0) and A1 astrocytes. FIG. 24C shows a dot plot of pan, A1-specific, and A2-specific astrocyte transcripts in A0 and A1 astrocytes, highlighting a stronger gene expression response in mature astrocytes. Dot size represents the percentage of cells that express a transcript, and color intensity represents the expression level of a transcript. FIG. 24D shows tSNE feature plots of reactive astrocyte transcripts. FIG. 24E shows that GFAP protein levels are stable in A0 and A1 CD49f⁺ hiPSC-astrocytes. Western blots for GFAP and b-actin, and quantification of protein levels, normalized to b-actin. Error bars show mean±standard deviation (n=5, 1-2 replicates per line). Dots correspond to 3 different lines. p-values were calculated using a two-tailed, paired t-test. FIG. 24F shows that TIMP1 level increases in CD49f⁺ hiPSC-astrocytes stimulated to A1. Western blots for TIMP1 and b-actin, and protein quantification, normalized to b-actin. Error bars show mean±standard deviation (n=5, 1-2 replicates per line). Dots correspond to 3 different lines. p-values were calculated using a two-tailed, paired t-test. Abbreviations: Imm.=immature; Trans.=transitioning.

FIG. 25 shows gating strategies for CD49 FACS. FIG. 25A shows representative flow-cytometry contour plot (with outliers shown) of the PI-stained negative control for the CD49f sort from mixed cultures. FIG. 25B shows plots for side scatter and forward scatter, duplets exclusions and live gates depicting the gating strategy for the CD49f sort from mixed cultures for 3 lines. FIG. 25C shows a representative flow-cytometry contour plot (with outliers shown) of the PI-stained negative control for the HepaCAM sort. FIG. 25D shows plots for side scatter and forward scatter, duplets exclusions and live gates depicting the gating strategy for the HepaCAM sort.

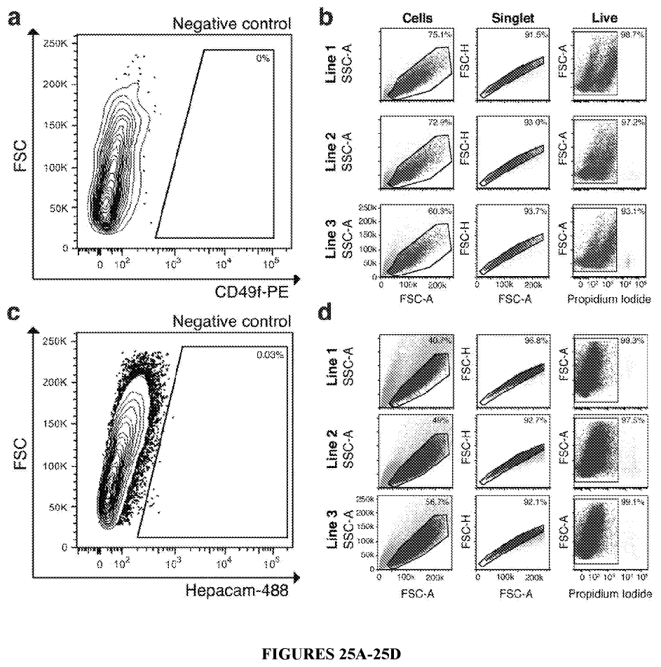

FIG. 26 shows that CD49f allows astrocyte isolation from cortical organoids. FIG. 26A shows representative immunofluorescence images of CD49f⁺, AQP4⁺, and GFAP⁺ cells with DAPI nuclei, showing that astrocytes are CD49f-positive in cryosections of hiPSC-derived oligocortical organoids. Scale bar, 10 μm. FIG. 26B shows a schematic of experimental design depicting astrocyte immunostaining and isolation from oligocortical organoids. FIG. 26C shows representative flow-cytometry contour plot (with outliers shown) of the CD49f sort from hiPSC-oligocortical organoids. FIG. 26D shows representative immunofluorescence images of GFAP⁺, AQP4⁺, with DAPI nuclei in the CD49f⁺ and CD49f⁻ fractions of the sort from oligocortical organoids, showing that astrocytes are enriched in the CD49f⁺ fraction. Scale bar, 100 μm. FIG. 26E shows percentages of GFAP⁺ cells and AQP4⁺ cells in the unsorted, CD49f⁺, and CD49f⁻ fractions of the sort, showing that AQP4⁺ and GFAP⁺ astrocytes are enriched in the CD49f⁺ fraction of the sort. Error bars show mean f standard deviation (n=1-2 independent differentiations). FIG. 26F shows representative flow-cytometry contour plot (with outliers shown) of the PI-stained negative control for the CD49f sort from dissociated oligocortical organoids. FIG. 26G shows plots for side scatter and forward scatter, duplets exclusions and live gates depicting the gating strategy for the CD49f sort dissociated oligocortical organoids.

FIG. 27 shows that CD49f⁺ hiPSC-astrocytes transcriptionally resemble ventral spinal cord astrocytes. See also FIG. 2. FIG. 27A shows hierarchical clustering of RNA-Seq data showing that CD49f⁺ iPSC-astrocytes (black) cluster closely with spinal cord-patterned iPSC-derived astrocytes from an independent study and away from forebrain-patterned astrocytes (GEO: GSE133489). Analysis is based on transcriptome-wide expression. CD49f⁺ samples consist of 3 different lines and 3 technical replicates per line. FIG. 27B shows a heat map of regional transcripts from RNA-seq data (OTX2, forebrain; NKX2-1, ventral forebrain; FOXG1, dorsal forebrain; HOXA3, spinal cord; NKX2-2, ventral spinal cord), as transcripts per million in CD49f⁺ hiPSC-astrocytes. The expression of ventral spinal cord markers is consistent with sonic hedgehog and retinoic acid patterning, used in the differentiation protocol provided herein. Samples consist of 3 lines and three technical replicates per line.

FIG. 28 shows that CD49f⁺ hiPSC-astrocytes do not express oligodendrocyte progenitor cell markers. Cells expressing oligodendrocyte progenitor cell markers PDGFRA and CSPG4 are enriched in the CD49f⁻ sorted fraction and do not overlap with ITGA6 expression. tSNE feature plots of CD49f (ITGA6) and OPC (PDGFRA, CSPG4) transcripts from unsorted, CD49f⁺ sorted, and CD49f⁻ sorted cells. All data from one control line (line 3, n=1).

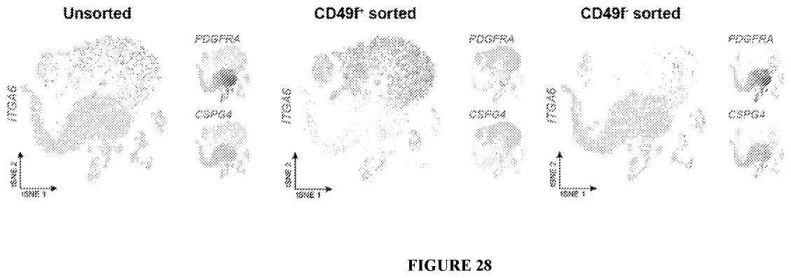

FIG. 29 shows that CD49f⁺ hiPSC-astrocytes express human-specific transcripts. tSNE feature plots of CD49f (ITGA6) and human-specific astrocyte transcripts LRRC3B, HSSD17B6, FAM198B, RYR3, STOX1, MRV11 from unsorted, CD49f⁺ sorted, and CD49f⁻ sorted cells. Cells expressing human-specific astrocyte transcripts are enriched in the CD49f⁺ fraction of the sort and overlap with ITGA6 expression. All data from one control line (line 3, n=1).

FIG. 30 shows that CD49f⁺ hiPSC-astrocytes express late pseudotime markers. FIG. 30A shows a tSNE plot of all astrocytes from unsorted, CD49f⁺ sorted, and CD49f⁻ sorted cells (n=12,061 astrocytes). FIG. 30B shoes a heatmap and corresponding tSNE feature plots of markers classified as early, middle, and late pseudotimes. Immature astrocytes (enriched in CD49f⁻ fraction) have higher expression of early pseudotime transcripts, while mature astrocytes (enriched in CD49f⁺ fraction) have higher expression of middle and late pseudotime transcripts. Abbreviations: Imm.=Immature.

FIG. 31 shows that CD49f sorting strategy fails to purify astrocytes from Aldh1/1_{eGFP} mouse whole brain. See also FIG. 4. FIG. 31A shows representative FACS plots of isolated cells from Aldh1/1^{eGFP} mouse whole brain. Cells were stained with CD49f antibody, isotype control, or null-stained control, and sorted. The sorting strategy removed red blood cells (not shown in these plots), followed by doublets, debris, and live gating (DAPI). FIG. 31B shows that compared to isotype control, CD49f⁺ cells yielded a distinct population of CD49f⁺ (population 2; P2), which were Aldh1/1^{eGFP} negative. FIG. 31C shows immunofluorescent analysis of sorted cells. P1 (Aldh1/1^{eGFP+} cells) were negative for CD49f, while P2 (CD49f⁺ cells) did not stain for the astrocyte reporter Aldh1/1^{eGFP}, suggesting that CD49f sorting strategy does not enrich for astrocytes from a mouse whole brain. P3 was negative for both markers (Aldh1/1^{eGFP-}CD49f⁻). Fluorescent images are also merged with phase images. FIG. 31D shows PCR analysis for oligodendrocytes (Mog), neurons (Snap25), microglia (Tmem119) and endothelial (Cd31) markers, showing that sorted cells from the CD49f⁺ fraction express Tmem119 and Cd31, but not GFAP.

FIG. 32 shows that IL-1α is secreted following IL-1β and TNFα stimulation. See also FIG. 5. CD49f⁺ hiPSC-derived astrocytes stimulated with TNFα and IL-1β secrete IL-1α. Concentration of IL-1α secreted in the supernatant of CD49f⁺ astrocytes that were left unstimulated or stimulated for 24 hours with TNFα and IL-1β. Concentrations are expressed in pg/ml and normalized to 1,000 cells. Different dots correspond to 3 different lines (n=6, 2 technical replicates per line). Error bars show mean±standard deviation. p-values were calculated using a paired two-tailed t-test.

FIG. 33 shows that CD49f⁺ hiPSC-astrocytes stimulated with TNFα, IL-1α, and C1q express A1 signature genes. See also FIG. 6. FIG. 33A shows that mRNA expression levels from RNA-Seq data as transcripts per million (TPM) of A1-specific transcripts (C3, SERPING1, GBP2, FBLN5, GGTA1P, FKBP5, PSMB8, SRGN, AMIGO2, UGT1A1) are upregulated in astrocytes stimulated with TNFα, IL-1α, and C1q (A1) compared to unstimulated astrocytes (A0). Different dots correspond to 3 different lines. Error bars show mean±standard deviation (n=3 independent lines). p-values were calculated using a two-tailed, paired t-test. FIG. 33B shows mRNA expression levels from RNA-Seq data as transcripts per million (TPM) of known astrocyte glutamate transporters (SLC1A3, SLC1A2) in unstimulated astrocytes (A0) and astrocytes stimulated with TNFα, IL-1α, and C1q (A1). Different dots correspond to 3 lines. Error bars show mean±standard deviation (n=3 independent lines). p-values were calculated using a two-tailed, paired t-test. FIG. 33C shows Western blots and electropherograms for EAAT2 and 13-actin, and quantification of protein levels for EAAT2 in A0 and A1-like astrocytes, normalized to 13-actin, from two independent experiments. EAAT2 protein levels are unchanged in A1-like astrocytes vs. A0 astrocytes. Different dots correspond to 3 lines. Error bars show mean±standard deviation (n=5, 1-2 replicates per line). p-values were calculated using a two-tailed, paired t-test. FIG. 33D shows mRNA expression levels from RNA-Seq data as transcripts per million (TPM) of lysosomal activity markers (LAMP1, LAMP2, RAB7A) in unstimulated astrocytes (A0) and astrocytes stimulated with TNFα, IL-1α, and C1q (A1). All markers are significantly downregulated upon A1 stimulation. Different dots correspond to 3 lines. Error bars show mean f standard deviation (n=3 independent lines). p-values were calculated using a two-tailed, paired t-test. FIG. 33E shows a dose-response curve of rodent astrocytes to ATP treatment. Error bars show mean±standard error of the mean. There was no alteration in response (no shift in curve) in response to ATP in rodent A1-reactive astrocytes when compared to control.

FIG. 34 shows that TNFα, IL-1α, and C1q are not toxic to neurons, while conditioned medium from A1-like CD49f⁺ hiPSC-astrocytes is toxic to mouse neurons. See also FIG. 7. FIG. 34A shows a schematic of neuronal cultures stimulated with inflammatory cytokines. FIG. 34B shows that direct treatment with TNFα, IL-1α, and C1q, in the absence of astrocytes, has no effect on the electrophysiological properties of hiPSC-neurons. Bar graphs showing the maximum number of evoked spikes per 1 second stimulus (n=10;6), the maximum firing frequency in hertz (Hz) (n=4;5), the half-width of the first spike (ms) (n=10;6), the maximum action potential height (mV) (n=10;6), the amplitude adaptation ratio between first and last action potential (n=3;5), and the sEPSC frequency (Hz) (n=5;3) in hiPSC-neurons at day 53 when cultured with or without TNFα, IL-1α, and C1q during days 33-53. Each dot represents an independent cell from that was recorded from. n=neurons co-cultured without cytokine cocktail; neurons co-cultured with cytokine cocktail. Error bars show mean±standard deviation. p-values were calculated using a two-tailed, unpaired t-test. FIG. 34C shows representative images of mouse neurons treated with A0 or A1 astrocyte conditioned media (CM) for 60 hours, showing caspase 3/7 and phase. A1 conditioned medium increases apoptosis in mouse neuronal cultures. Scale bar, 100 μm. Abbreviations: CM=conditioned medium. FIG. 34D shows time course analysis and quantification (60 hours) of apoptotic caspase 3/7⁺ cells in mouse neuronal cultures after stimulation with A0 or A1 CD49f⁺ hiPSC astrocyte conditioned media. Error bars represent the standard error of the mean (n=6-12 replicates per line), indicating a neurotoxic effect of A1 conditioned medium. p-values were calculated using a two-way ANOVA. FIG. 34E shows time course analysis and quantification (60 hours) of the caspase 3/7 integrated intensity (normalized to confluence) in mouse neurons during treatment with TNFα, IL-1α, and C1q. Error bars represent the standard error of the mean (n=6 replicates per line), demonstrating no direct effect of the cytokine cocktail on neuronal apoptosis. p-values were calculated using a two-way ANOVA.

FIG. 35 shows that iPSC cell lines undergo a rigorous quality check. The CoA for iPSC line 051121-01-MR-017 describing results of quality check including a sterility check, mycoplasma testing, karyotyping, and a pluripotency check is shown.

### DETAILED DESCRIPTION OF THE INVENTION

iPSC lines generated using a fully automated reprogramming platform that has been demonstrated to reduce line-to-line variability were used. A differentiation protocol developed to generate oligodendrocytes within mixed cultures, which also include neurons and astrocytes in serum-free conditions, was leveraged.

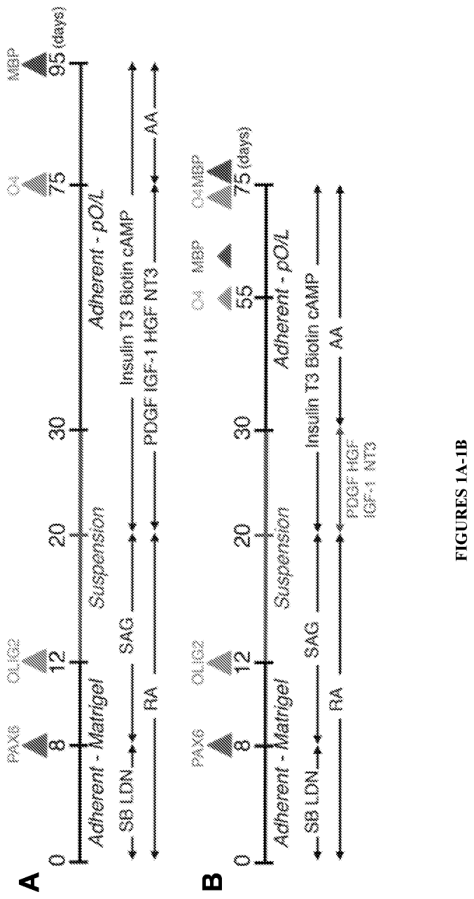

A robust, fast, and reproducible differentiation protocol to generate mixed populations of human oligodendrocytes and astrocytes from PSCs using a chemically defined, growth factor-rich medium was developed. The protocol provided herein mimics oligodendrocyte differentiation during development. Within 8 days PSCs differentiate into PAX6⁺ neural stem cells, which give rise to OLIG2⁺ progenitors by day 12. OLIG2⁺ cells become committed to the oligodendrocyte lineage by co-expressing NKX2.2 around day 18, and then differentiate to early OPCs by up-regulating SOX10 and PDGFRα around day 40. Late OPCs expressing the sulfated glycolipid antigen recognized by O4 antibody (O4+) appear around day 50, and reach 40-70% of the cell population by 75 days of differentiation. O4⁺ oligodendrocyte progenitor cells can be isolated by cell sorting for myelination studies, or can be terminally differentiated to mature MBP⁺ oligodendrocytes. The timeline of the differentiation protocol provided herein is shown in FIG. 1.

A wealth of evidence implicates astrocytes in CNS disease pathology, and efforts to identify novel therapeutic targets have increasingly focused on astrocytes. Given that many of today's incurable diseases, such as AD, PD, and MS, are specific to humans and critical interspecies differences are evident, human patient-specific models are a necessary tool to complement traditional animal models for elucidating pathogenic mechanisms and developing effective treatments. Here, hiPSC-astrocytes purified using the surface marker CD49f were demonstrated to be a compelling tool for modeling primary human astrocytes and for studying astrocytic function and dysfunction in vitro. CD49f is a laminin receptor and has been previously reported as a marker for stem cells, including glioblastoma and other cancer stem cells. As described herein, CD49f can also be used for enriching primary fetal human astrocytes and is present in astrocytes from adult human brains in both healthy individuals and neurological disease patients. Importantly, CD49f is a reactivity-independent marker (expressed in both unstimulated and reactive astrocytes), making it ideal for purification strategies in the study of neurodegenerative disease. Established markers for isolating rodent cells, such as HepaCAM, are ineffective for hiPSC-astrocytes, except following prolonged culture to enable complete maturation—just as many available differentiation protocols do not achieve a maturation state equivalent to in vivo adult cells. Indeed, transcriptomic analysis of hiPSC-astrocytes described herein showed expression of both immature and mature markers, but single-cell analysis determined that the differentiation yields distinct populations of both immature and mature astrocytes. It should be noted that, due to the patterning during the initial differentiation step with the caudalizing and ventralizing agents retinoic acid and sonic hedgehog, the transcriptomic profile of hiPSC-astrocytes is very similar to that of ventral spinal cord astrocytes. In line with findings about regionally specified astrocytes, data described herein suggest that regional heterogeneity exists, stemming from cell-intrinsic developmental differences and can be recapitulated in vitro, further emphasizing the usefulness of this resource. Nonetheless, it is important to point out that CD49f is not a spinal cord—specific marker.

Astrocytes from cortical organoids and from fetal brain were successfully isolated, as described herein, and CD49f⁺ astrocytes were identified in sections of the subventricular zone and pre-frontal cortex of adult brains. Additionally, independent transcriptomic analyses showed that ITGA6 expression is higher in human forebrain astrocytes than in spinal cord astrocytes.

CD49f⁺ astrocytes generated through the differentiation protocol provided herein achieve within 75 days a maturation stage comparable to that of astrocytes derived from organoids at much later time points, making the strategy described herein optimal for functional studies in vitro. However, it is difficult to directly compare these two protocols, as they use different media formulations and patterning agents, and because the cells are grown in a 2D vs. 3D format. Interestingly, scRNA-seq analysis also revealed that CD49f⁺ astrocytes consist of multiple astrocyte subclusters with varying expression of genes involved in lipid biosynthesis, neurotransmitter uptake, gliogenesis, antigen presentation, neural development, cell motility and many other important biological processes. Further investigations into these subclusters may provide valuable insights into the functional heterogeneity of astrocytes.

Functional assays confirmed CD49f⁺ astrocyte cultures as a strong platform for disease modeling and for investigating neuronal support, engulfment of debris, glutamate uptake, response to inflammatory stimuli, and neurotoxicity. In the context of neuroinflammation, CD49f⁺ astrocytes were shown to respond to pro-inflammatory stimuli by secreting typical chemokines and cytokines. Interestingly, major differences between stimulation with TNFα, IL-1α, C1q (driving the A1 phenotype vs. TNFα and IL-1β, which are typically released by microglia in neurodegenerative diseases, were not observed. The similarity in cytokine release after stimulation with either cocktail is likely explained by the dominant effect of TNFα, present in both, and by the fact that IL-1β is released by astrocytes upon stimulation with TNFα and IL-1β, triggering autocrine signaling. Transcriptomic profiles of hiPSC-derived A0 and A1-like reactive astrocytes are available as a resource through a searchable online database (nyscfseq.appspot.com). This transcriptomic analysis revealed that human A1-like reactive astrocytes largely conserve the A1 signature identified in rodent cells and indicates loss of function related to phagocytosis and glutamate uptake. This underscores the value of methods provided herein for isolating and generating patient-specific astrocytes to better understand the pathogenic mechanisms linked to human A1 neurotoxicity in neurological diseases. Furthermore, single-cell RNA-Seq analysis highlighted differences in response to TNFα, IL-1α, C1q treatment based on the developmental stages of hiPSC-astrocytes (immature, transitioning, mature). This likely reflects a difference in the response of astrocytes to infection/injury/disease across different stages of development, which can be further investigated thanks to the hiPSC-based platform described herein.

As discussed herein, CD49f was determined to be a reactivity-independent, astrocyte-specific cell surface antigen that is present at all stages of astrocyte development in hiPSC-derived cultures. Astrocytes isolated with this marker recapitulate in vitro critical physiological functions, and following inflammatory stimulation become reactive, dysfunctional, and toxic, triggering neuronal death—opening a window for the study of their role in neurodegenerative diseases. hiPSC-derived CD49f⁺ astrocytes can be used to advance a “clinical trials in a dish” approach to drug discovery, and incorporating hiPSC-based models in the preclinical phase of drug development will improve the success of drug discovery for neurological diseases with a high unmet need. The strategy to purify hiPSC-derived astrocytes using CD49f provided herein will facilitate disease modeling with patient-specific to better understand pathogenic mechanisms of astrocyte reactivity in infection, injury, and developmental and degenerative diseases.

Signaling pathways for manipulation to generate mixed oligodendrocytes/astrocyte populations were selected based on knowledge gained from studies of rodent spinal cord embryonic development. In vitro, RA and SHH signaling mimic the pMN environment, inducing differentiation of the PSCs to OLIG2 progenitor cells. In cultures provided herein, SHH signaling is activated through a Smoothened agonist instead of the human recombinant SHH protein. Combining RA and SHH signaling with activin/nodal/TGFβ inhibition (through SB431542) and BMP4 inhibition (through LDN193189), generated the highest yield of OLIG2⁺ progenitors. Due to the synergistic action of SB431542 and LDN193189, more than 70% of the cells express OLIG2 at day 12. This is a key difference between the protocol provided herein. Inhibition of both activin/nodal/TGFβ signaling and BMP signaling is also referred to as “dual SMAD inhibition.”

There are several other important differences between methods provided herein and previously published protocols. For example, neural induction in methods provided herein was begun with dual SMAD inhibition in adherent, as opposed to suspension, cultures. Using this approach, methods provided herein started with only 10,000 cells/cm² at day 0, and yet achieved a great expansion of neural progenitors, and ultimately an abundant generation of OPCs. In addition, the optimal concentration of RA in our hands was found to be about 100 nM, which is one hundred times lower than the concentration used by other groups. Further, induction with RA alone, without exogenous SHH, surprisingly generated a large population of OLIG2⁺ cells. An agonist of Smoothened was confirmed as an efficient replacement for SHH and indeed showed superior efficacy. Moreover, methods provided herein, remarkably, are the first to show OLIG2 induction through RA in the absence of fibroblast growth factor (FGF) signaling. The combination of RA and FGF signaling is known to promote OLIG2 expression during chicken development and has been used for in vitro differentiation of both human ESCs and iPSCs. A recent study suggested that basic FGF (bFGF) is important to the specification of oligodendrocytes of ventral forebrain origin, while it inhibits neuronal differentiation during the specification of oligodendrocytes from the spinal cord. Nonetheless, a high yield of OPCs was achieved in the absence of any exogenous FGF during in vitro differentiation in methods provided herein. Finally, the transition from adherent cultures to suspension cultures of cell spheres at day 12 proved to be an important step to enrich the population of OLIG2⁺ cells and to restrict differentiation of cultures to the oligodendrocyte lineage.

Described herein is a method of generating OLIG2+ OPCs by first preparing PSC colonies. OLIG2+ OPCs are further differentiated to O4+ OPCs and CD49f⁺ astrocytes generated within the O4+ OPC population are sorted and isolated from the mixed population.

As such, in one embodiment, the invention provides a method of generating and isolating an astrocyte. The method includes: a) generating a mixed population of cells by culturing an SC under conditions to induce neuronal differentiation; b) selecting for a CD49f+ cell from the mixed population of cells; and c) isolating the CD49f+ cell from the mixed population of cells, wherein the CD49f+ is a CD49f+ astrocyte, thereby generating and isolating the astrocyte.

In a related embodiment, the invention provides invention provides a method for isolating an astrocyte from a mixed population of cells. The method includes: a) selecting for a CD49f+ cell from the mixed population; and b) sorting and isolating the CD49f+ cell from the mixed population, wherein the CD49f+ cell is a CD49f+ astrocyte, thereby isolating the astrocyte.

To generated OLIG2+ OPCs, PSCs are seeded (plated) at low density and grown in an adherent culture for about 1-2 days. “Low density” means about 8,000 to about 11,000 cells/cm². Cells are preferably seeded at about 9,500 to about 10,500 cells/cm², more preferably at about 10,000 cells/cm². After 1-2 days, the PSCs form colonies, which are preferably about 75 μm to about 300 μm in diameter, more preferably about 100 μm to about 250 μm in diameter.

The term “PSCs” has its usual meaning in the art, i.e., self-replicating cells that have the ability to develop into endoderm, ectoderm, and mesoderm cells. Preferably, PSCs are hPSCs. PSCs include ESCs and iPSCs, preferably hESCs and hiPSCs. PSCs can be seeded on a surface comprising a matrix, such as a gel or basement membrane matrix. A preferable matrix is the protein mixture secreted by Engelbreth-Holm-Swarm (EHS) mouse sarcoma cells, sold under trade names including Matrigel™, Cultrex™, and Geltrex™. Other suitable matrices include, without limitation, collagen, fibronectin, gelatin, laminin, poly-lysine, vitronectin, and combinations thereof.

The medium in which PSCs are cultured preferably comprises an inhibitor of rho-associated protein kinase (ROCK), for example, GSK269962, GSK429286, H-1152, HA-1077, RKI-1447, thiazovivin, Y-27632, or derivatives thereof.

The PSC colonies are then cultured in a monolayer to confluence in a medium comprising a low concentration of RA, at least one inhibitor of TGFβ signaling, and at least one inhibitor of BMP signaling, wherein the first day of culturing in this medium is day 0. A “low concentration of RA” is about 10 nM to about 250 nM. The concentration of RA is preferably about 10 nM to about 100 nM, or about 25 nM to about 100 nM, or about 20 nM, 30 nM, 40 nM, 50 nM, 60 nM, 70 nM, 80 nM, 90 nM, and preferably, about 100 nM or less. Inhibitors of TGFβ signaling include, for example, GW788388, LDN193189, LY2109761, LY2157299, and LY364947. A preferred inhibitor of TGFβ signaling is the small molecule SB431542. Inhibitors of BMP signaling include, for example, DMH1, dorsomorphin, K02288, and Noggin. A preferred inhibitor of BMP signaling is the small molecule LDN193189.

Cells reach confluence and express PAX6 at about day 8, at which point the confluent cells are cultured in a medium comprising SHH or an agonist of Smoothened, and a low concentration of RA. Agonists of Smoothened include, for example, SAG and purmorphamine. The SHH can be recombinant human SHH. Preferably, the medium lacks SHH. The transition from PSCs to OLIG2⁺ progenitors is associated with massive proliferation causing the cultures to become overconfluent, resulting in the cells forming three-dimensional structures, ideally by about day 12. “Overconfluent” means that the cells begin piling up on one another, such that not all cells are in complete contact with the culture surface, and some cells are not in contact with the culture surface at all, but are only in contact with other cells. Preferably, at least about 50/6, 60%, or 70% of the overconfluent cells are OLIG2+ by about day 12.

If the OLIG2+ cells are to be further differentiated to O4+ cells, the overconfluent cells are lifted from the culture surface, which allows the formation of cell aggregates or spheres. OLIG2⁻ cells do not form aggregates, thus this process enriches for the OLIG2+ population, and OLIG2⁻ cells are eliminated gradually during subsequent media changes. For purposes of the present invention, the terms “aggregate” and “sphere” are used interchangeably and refer to a multicellular three-dimensional structure, preferably, but not necessarily, of at least about 100 cells.

Lifting can be performed mechanically, with a cell scraper or other suitable implement, or chemically. Chemical lifting can be achieved using a proteolytic enzyme, for example, collagenase, trypsin, trypsin-like proteinase, recombinant enzymes, such as that sold under the trade name Tryple™, naturally derived enzymes, such as that sold under the trade name Accutase™, and combinations thereof. Chemical lifting can also be done using a chelator, such as EDTA, or a compound such as urea. Mechanical lifting or detachment offers the advantage of minimal cell death, however it produces aggregates of variable size, thus suitable spheres need to be selected through a manual picking process. Good spheres are defined as those having a round-shape, golden/brown color, with darker core and with a diameter between about 300 μm and about 800 μm. Detaching the cells using chemical methods, such as enzymatic digestion predominantly produces spheres that are appropriate for further culture. Therefore, manual picking of spheres is not required, and the detachment steps can be adapted for automation and used in high throughput studies. However, enzymatic digestion increases cell death, resulting in a lower number of spheres.

Further provided herein is a method of generating O4+ OPCs from OLIG2+ OPCs. Three-dimensional aggregates of OLIG2+ OPCs are cultured in suspension in a medium comprising a Smoothened agonist and a low concentration of RA for about 8 days. The OLIG2+ OPCs can be generated a method of the invention, for example, as described above, or by other methods known in the art. After about 8 days in the medium comprising the Smoothened agonist and RA, the medium is changed to one comprising PDGF, HGF, IGF-1, and NT3, and optionally, insulin (preferably about 10 μg/ml to about 50 μg/ml, more preferably about 25 μg/ml), T3 (preferably about 20 ng/ml to about 100 ng/ml, more preferably about 60 ng/ml), biotin (preferably about 50 ng/ml to about 150 ng/ml, more preferably about 100 ng/ml), and/or cAMP (preferably about 100 nM to about 5 μM, more preferably about 1 RM). The medium preferably lacks bFGF and epidermal growth factor (EGF). If OLIG2+ cells are generated by the method of the invention, culture in suspension preferably begins on about day 12, and culture in the medium comprising PDGF, HGF, IGF-1, and NT3 preferably begins on about day 20.

After about 10 days in suspension in the medium comprising PDGF, HGF, IGF-1, and NT3, the cell aggregates are plated in an adherent culture at a density of about 2 spheres/cm². (This is preferably at about day 30 where the method started on day 0 with PSCs cultured in a medium comprising RA, at least one inhibitor of TGFβ signaling, and at least one inhibitor of BMP signaling.) The surface on which the cell aggregates are plated and cultured can comprise an extracellular matrix protein (e.g., collagen, fibronectin, laminin) and/or a positively charged poly-amino acid (e.g., poly-arginine, poly-lysine, poly-ornithine). Preferably the surface comprises laminin and/or poly-ornithine.

Upon plating the cell aggregates, the medium comprising PDGF, HGF, IGF-1, and NT3 can be continued (Option A), or a medium comprising AA and lacking growth factors (e.g., PDGF, HGF, IGF-1, NT3, bFGF, and/or EGF) can be used (Option B). The medium comprising AA can optionally comprise insulin, T3, biotin, and/or cAMP. Cells cultured in the medium comprising PDGF, HGF, IGF-1, and NT3 are optimally O4+ by about 45 days after plating. Preferably, at least about 35%, 40%, 45%, 50%, 55%, 60%, 65%, 70%, 75%, or 80% of these cells are O4+ by about 45 days after plating (day 75). Cells cultured in the medium comprising AA are optimally O4+ by about 25 days after plating. Preferably, at least about 20%, 25%, 30%, 35%, or 40% of these cells are O4+ by about 25 days after plating (day 55). Preferably, at least about 30%, 35%, 40%, 45%, 50%, 55%, or 60% of these cells are O4+ by about 33 days after plating (day 63). Preferably, at least about 35%, 40%, 45%, 50%, 55%, 60%, 65%, 70%, or 75% of these cells are O4+ by about 45 days after plating (day 75).

Mature oligodendrocytes expressing myelin basic protein can be generated by culturing the O4+ OPCs in the absence of PDGF, HGF, IGF-1, and NT3 for about three weeks, until cells are MBP+. Preferably, at least about 20%, 25%, 30%, 35%, 40%, or 45% of the O4+ OPCs are MBP+ after about 20 days in culture in the medium lacking PDGF, HGF, IGF-1, and NT3. This occurs on about day 95 for “Option A” cells, and on about day 60 for “Option B” cells. Culturing “Option B” cells until at least about day 75 results in a higher efficiency of MBP+ expressing cells.

At day 60-80, spheres and the cells migrating out of the spheres may be dissociated and sorted for CD49f-positive cells. After the sort, sorted cells may be further cultured in glial medium (see Table 10 below) to generate an isolated CD49f-positive astrocyte population.

In various embodiments, CD49f⁺ astrocyte isolation may be performed by FACS using a fluorescently labeled CD49f antibody. While this technique is illustrated in the Examples, the invention is in no way limited to use of FACS. It will be appreciated that isolating CD49f⁺ astrocytes may be accomplished using a number of techniques generally known in the art. For example, isolation may be accomplished using a number isolation techniques including flow cytometry, immunoseparation, immunocapture and the like. In some embodiments, a CD49f specific antibody may be conjugated to a solid substrate for isolation. As used herein, a solid substrate includes any solid support, such as a bead, slide or other type of solid support. In one embodiment, isolation is performed using a bead conjugated to a CD49f specific antibody. The bead may optionally be magnetic allowing for separation of a CD49f⁺ astrocyte bound to the bead via exposure to a magnetic source.

Also provided herein is a kit for generation and/or isolation of a CD49f⁺ astrocyte. In embodiments, the kit includes an antibody that selectively binds CD49f; and optionally a reagent for generating, culturing and/or isolating a CD49f+ astrocyte. In various embodiments, the antibody is optionally labeled with a fluorescent moiety and/or a binding moiety. The antibody may also be conjugated to a solid support, such as bead.

The invention may utilize a variety of techniques for conjugating or otherwise forming an attachment between two molecules. For example, a surface or molecule may be functionalized by addition of a functional group. A functional group may be a group capable of forming an attachment with another functional group. For example, a functional group may be biotin, which may form an attachment with streptavidin, another functional group. Exemplary functional groups may include, but are not limited to, aldehydes, ketones, carboxy groups, amino groups, biotin, streptavidin, nucleic acids, small molecules (e.g., for click chemistry), homo- and hetero-bifunctional reagents (e.g., N-succinimidyl(4-iodoacetyl) aminobenzoate (STAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-mafeimidomethyl)-cyclohexane-1-carboxylate (SMCC) and 6-hydrazinonicotimide (HYNIC), and antibodies. In some instances, the functional group is a carboxy group (e.g., COOH). As such, the kit of the present invention may include a functionalized reagent.

In various aspects, the kit optionally includes one or more cell culture reagents for generating a CD49f+ astrocyte, using for example a culture method provided herein. As such, the kit may include one or more components of a cell culture medium. In embodiments, the cell culture medium is a culture medium as set forth in any one of Tables 5-11 or any combination thereof.

Methods provided herein also encompass generation of organoids or other mixed population of cells by co-culturing an isolated Cd49f+ with a different cell type, such as a neuronal cell. Isolated astrocytes, as well as organoids or other mixed population of cells, may be used as therapeutic agents, as well as models for studying disease pathology, drug screening, and the like.

In one embodiment, provided herein is a model system for a neurological disease, preferably a neurodegenerative disease or disorder. In one aspect, the model system includes an astrocyte differentiated from an iPSC derived from a subject having a neurological disease. The model system can further include a non-human mammal into which the astrocyte has been transplanted. In embodiments, the non-human mammal is a mouse or a rat. Model systems provided herein can be used to study neurological diseases or disorders, including understanding underlying mechanisms and defining therapeutic targets.

Also provided herein are methods for treating and/or preventing a neurological disease or disorder in a subject by generating astrocytes via the method of the invention; and administering an effective amount of the cells to the subject.

Alongside its potential for autologous cell transplantation, iPSC technology is emerging as a tool for developing new drugs and gaining insight into disease pathogenesis. The methods and cells of the invention will aid the development of high-throughput in vitro screens for compounds that inhibit or prevent a neurological disease or disorder.

The cells, systems, and methods of the invention can also be useful for studying neurological diseases.

In an embodiment, unbiased screening for surface molecules in mixed cultures that include oligodendrocytes, neurons, and astrocytes identified CD49f as a novel marker for purifying hiPSC-astrocytes from neurons and oligodendrocyte lineage cells through FACS. CD49f, encoded by the gene ITGA6, is a member of the integrin alpha chain family of proteins and interacts with extracellular matrices, including laminin. The Brain RNA-Seq database (www.brainrnaseq.org/) confirmed that ITGA6 expression is higher in human fetal and mature astrocytes compared to neurons, oligodendrocytes and microglia. ITGA6 is also expressed in endothelial cells, but hiPSC differentiations toward the ectodermal lineage produce neural populations with no mesodermal/endothelial cell contribution.

As provided herein, CD49f can be used to sort astrocytes from monolayer cultures and 3D cortical organoids containing oligodendrocyte lineage cells (i.e., oligodendrocyte progenitor cells, immature and mature oligodendrocytes), neural progenitors, and neurons. CD49f astrocytes can express typical markers, display similar gene expression profiles to human primary astrocytes and perform critical astrocyte functions in vitro. Specifically, they support neuronal growth and synaptogenesis, generate spontaneous Ca²⁺ transients, respond to ATP, perform glutamate uptake, and secrete inflammatory cytokines in response to inflammatory stimuli.

Moreover, CD49f⁺ hiPSC-astrocytes acquire an A1-like reactive phenotype upon stimulation with TNFα, IL-1α, and C1q, while maintaining CD49f expression. Transcriptome analysis revealed a conserved A1 signature with similar losses of function as reported in rodent cells, including impaired glutamate uptake, phagocytosis, and support of neuronal maturation. Importantly, rodent and hiPSC-derived neurons treated with A1 conditioned medium show significant increases in apoptosis, providing a human in vitro model for A1-driven neurotoxicity. Single-cell transcriptome analysis revealed slightly different A1 profiles in astrocytes at different maturation stages—suggesting that disease responses may change at different stages of development and during aging.

Accordingly, provided herein is a novel human-based platform to model astrocytes in vitro, in which CD49f⁺ hiPSC-derived astrocytes from early stage organoids and monolayer cultures can be used to study reactive states and interrogate their role in neurodevelopmental and neurodegenerative diseases.

As used in this specification and the appended claims, the singular forms “a,” “an,” and “the” include plural referents, unless the context clearly dictates otherwise. The terms “a” (or “an”), as well as the terms “one or more,” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology, P-S. Juo, (2d ed. 2002) can provide one of skill with general definitions of some terms used herein.

Units, prefixes, and symbols are denoted in their Système International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

By “subject” or “individual” or “patient” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, pigs, and so on.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a neurological disease or disorder, according to the methods provided herein if the patient shows, e.g., total, partial, or transient alleviation or elimination of symptoms associated with the disease or disorder.

“Prevent” or “prevention” refers to prophylactic or preventative measures that prevent and/or slow the development of a targeted pathologic condition or disorder. Thus, those in need of prevention include those prone or susceptible to the disease or disorder. In certain embodiments, a neurological disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

The following examples are provided to further illustrate the advantages and features of the present invention, but they are not intended to limit the scope of the invention. While these examples are typical of those that might be used, other procedures, methodologies, or techniques known to those skilled in the art may alternatively be used.

### Example 1

### Differentiation of Oligodendrocytes from hESC Lines

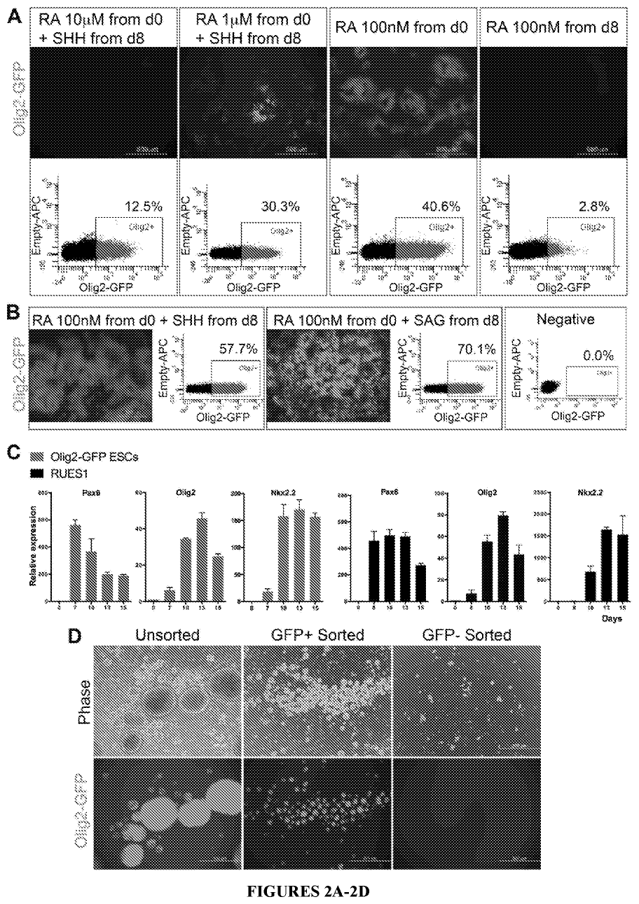

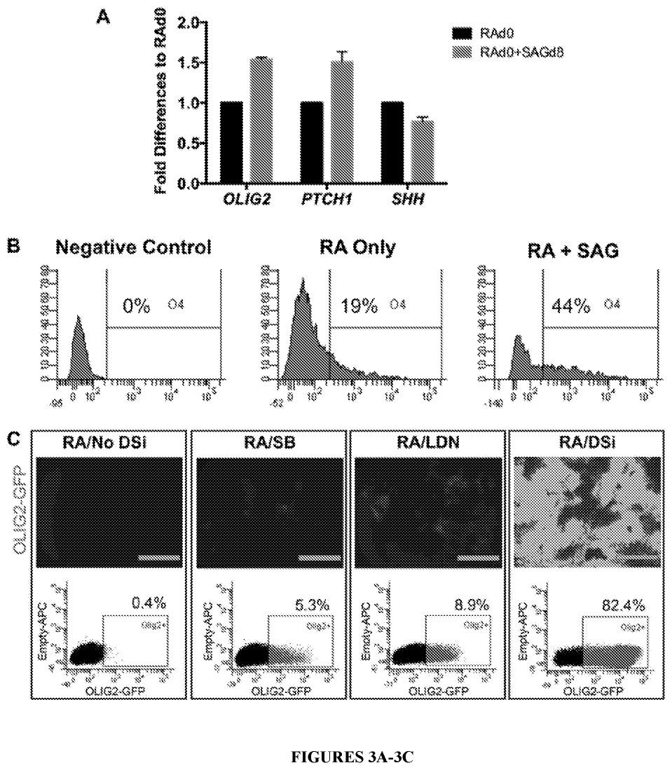

An OLIG2-GFP knock-in hESC reporter line was used to track OLIG2⁺ progenitors by live fluorescent imaging. First, PAX6⁺ cells were induced using dual inhibition of SMAD signaling in adherent cultures. Next, to mimic the embryonic spinal cord environment, different concentrations of RA and/or SHH were applied at various times and quantified OLIG2-GFP expression through flow cytometry (FIG. 2A). Application of 100 nM RA from the beginning of induction generated 40.6% of OLIG2⁺ progenitors, whereas addition of SHH at 100 ng/ml from day 8 increased the yield to 57.7% (FIG. 2B). Interestingly, cells without exogenous SHH during the first 12 days, showed an upregulation of SHH mRNA (FIG. 3A) and differentiated to O4⁺ cells, although at lower efficiency compared to cells treated with SHH (FIG. 3B).

Recombinant human SHH protein was then replaced with SAG, which increased the yield further to 70.1% OLIG2⁺ progenitors (FIG. 2B). At day 12, cells were detached and placed into low-attachment plates to promote their aggregation into spheres. The minimum number of cells required to form a sphere was at least 100 cells, and we noted that the majority of cells in the spheres were GFP⁺. To investigate this further, day 12 cultures were sorted for GFP, and it was observed that only the GFP⁺ cells formed aggregates (FIG. 2C). This suggests that the aggregation step alone provides enrichment for the OLIG2⁺ population.

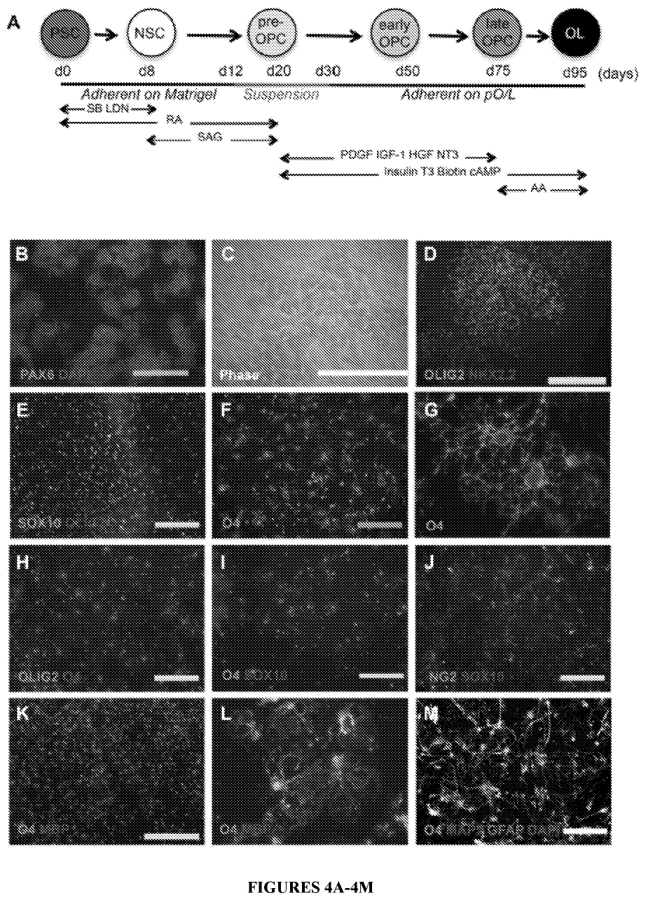

Next, the initial steps towards the generation of OLIG2⁺ progenitors were validated by differentiating a second hESC line (RUES1), and comparing the transcript levels of PAX6, OLIG2, and NKX2.2 by qRT-PCR. The upregulation of these transcription factors followed a similar temporal pattern to that of the OLIG2-GFP line, with PAX6 induction around day 7, OLIG2 peak around day 13, and sustainably high levels of NKX2.2 after day 10 (FIG. 2D). Based on these results, the non-genetically modified RUES1 line was used to develop the following steps of the protocol, from OLIG2⁺ progenitors to MBP⁺ mature oligodendrocytes (FIG. 4A). PAX6⁺ cells arose at day 7, and by day 12 they arranged into multilayered structures (FIG. 4B, 4C). From day 12 to day 30, cells were grown as spheres and then plated onto poly-L-ornithine/laminin (pO/L)-coated dishes for the remainder of the protocol.

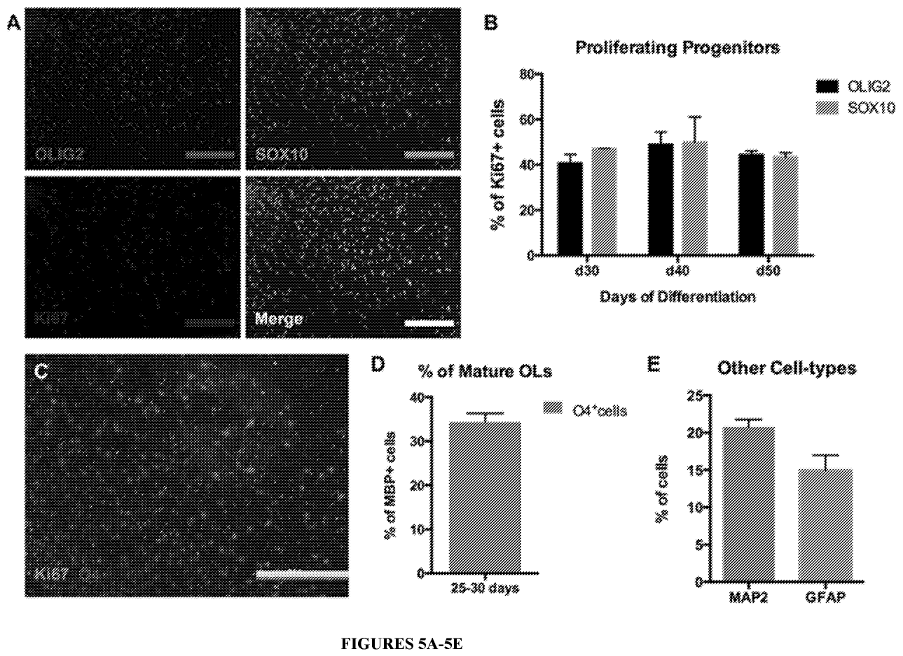

To promote maturation toward the O4⁺ stage, PDGF-AA, HGF, IGF1, and NT3 were added to the culture medium from day 20 onward. OLIG2⁺ progenitors upregulated NKX2.2, then SOX10, and finally matured to late OPCs identified by O4 live staining, and by their highly ramified processes (FIG. 4D-4G). O4⁺ OPCs expressing OLIG2, SOX10 and NG2 (FIG. 4H-4J), appeared as early as day 50 and their numbers increased dramatically around day 75. During the differentiation, 40-50% of progenitor cells were proliferative, as indicated by Ki67 staining. However, the highly ramified O4⁺ cells did not divide in vitro (FIG. 5A-5C). Additionally, 34±4% of O4⁺ OPCs differentiated into MBP⁺ mature oligodendrocytes after growth-factor withdrawal from the medium for at least two weeks (FIG. 4K-4L, FIG. 5D). These cultures also consisted of other cell-types, namely 15±2% GFAP⁺ astrocytes and 20±2% MAP2⁺ neurons of total cells respectively (FIG. 4M, FIG. 5E).

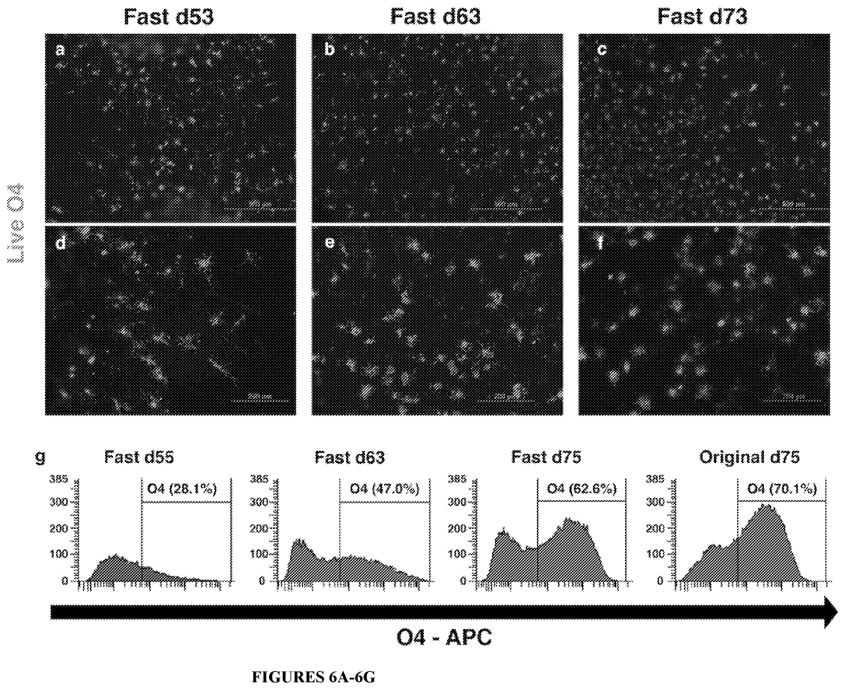

An alternative strategy to generate approximately 30% O4⁺ cells after only 55 days of culture was also used, significantly reducing the length and costs of differentiation. The mitogens PDGF, NT3, IGF-1 and HGF were withdrawn from the medium as early as at day 30, when the selected spheres were seeded. This resulted in the appearance of O4⁺ cells at day 55. The cultures were continued to increase the frequency of O4⁺ cells to levels comparable to the longer protocol (FIG. 6).

As shown in Table 1, O4 efficiencies ranged from 28% to 80% with nine different PSC lines, and the average was greater than 60% in four lines. Cells were stained with O4 antibody and analyzed by flow cytometry. One hESC line (RUES1) and eight hiPSC lines were tested. Technical replicates were performed using different batches of each line, at different passages. Results are also expressed as mean percentages±SEM.

TABLE 1
Percentages of O4⁺ OPCs After ~75 days of Differentiation

| Cell line | N | O4⁺ (%) | Mean ± SEM (%) |
|---|---|---|---|
| 102 | 4 | 40, 71, 72, 74 | 61.8 ± 7.6 |
| 104 | 4 | 55, 61, 61, 68 | 61.3 ± 2.7 |
| 107 | 1 | 48 | |
| 109 | 3 | 55, 60, 70 | 61.7 ± 4.2 |
| 110 | 3 | 29, 37, 73 | 46.2 ± 13.7 |
| 111 | 2 | 46, 47 | 46.4 ± 0.4 |
| 130 | 4 | 43, 47, 58, 76 | 56.1 ± 7.2 |
| 197 | 1 | 28 | |
| RUES1 | 5 | 36, 54, 68, 78, 80 | 62.9 ± 8.2 |

N = number of technical repeats.

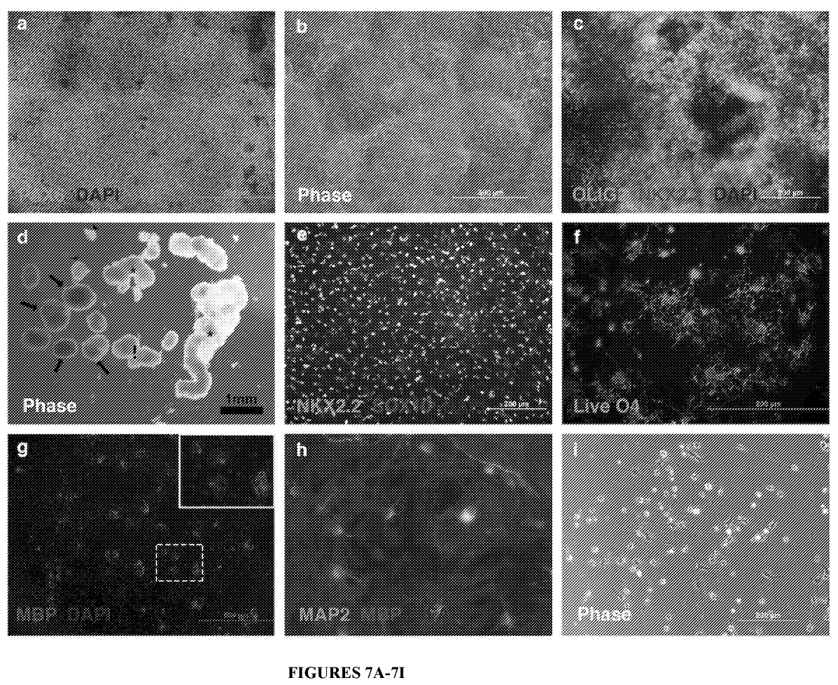

In both strategies, the withdrawal of the mitogens drives the terminal differentiation of OPCs to oligodendrocytes expressing MBP, although MBP⁺ cells do not align with axon fibers under these culture conditions (FIG. 7G, 7H). For myelination studies, O4⁺ OPCs can be purified through FACS and transplanted in vivo. O4⁺ cells can also be cryopreserved immediately after sorting and thawed 24-48 hours prior transplantation.

As described above, at various stages of the protocol, cultures were checked for the expression of appropriate markers by either qRT-PCR or immunofluorescence. When performing immunofluorescent analysis of the cultures at day 8 for PAX6 (FIG. 7A) and at day 12 for OLIG2 and NKX2.2 (FIG. 7C), the frequencies should be greater than 90% for the PAX6⁺, 70% for OLIG2⁺, and 30% for the OLIG2⁺/NKX2.2+ cells. By day 40-50, SOX10 should be expressed and should co-localize with OLIG2 and NKX2.2 (FIG. 7E). From day 50 to 75, live O4 staining can be performed to detect the appearance of O4⁺ cells and their expansion (FIG. 6A-6F, FIG. 7F). In human development, OPCs are characterized by PDGFRα and NG2 expression, followed by expression of O4. Underculture conditions described herein, by day 75, most O4⁺ cells have lost PDGFRα but have retained NG2 expression. At this stage no residual pluripotent cells were observed in culture.

Finally, O4⁺ cells can either be isolated via FACS or further differentiated to MBP⁺ oligodendrocytes (FIG. 7G, 7H). Other cell-types also exist in day 75 cultures, although at lower percentages. GFAP⁺ cells were generally found in about 15% of the total cell population and about 20% βIII-Tubulin⁺ cells (FIG. 7H). After the final differentiation step, when cells are cultured in Glial Medium for 2 weeks, about 35% of the O4⁺ cells should also express MBP.

### Example 2

### Differentiation of Oligodendrocytes from PPMS-iPSC Lines

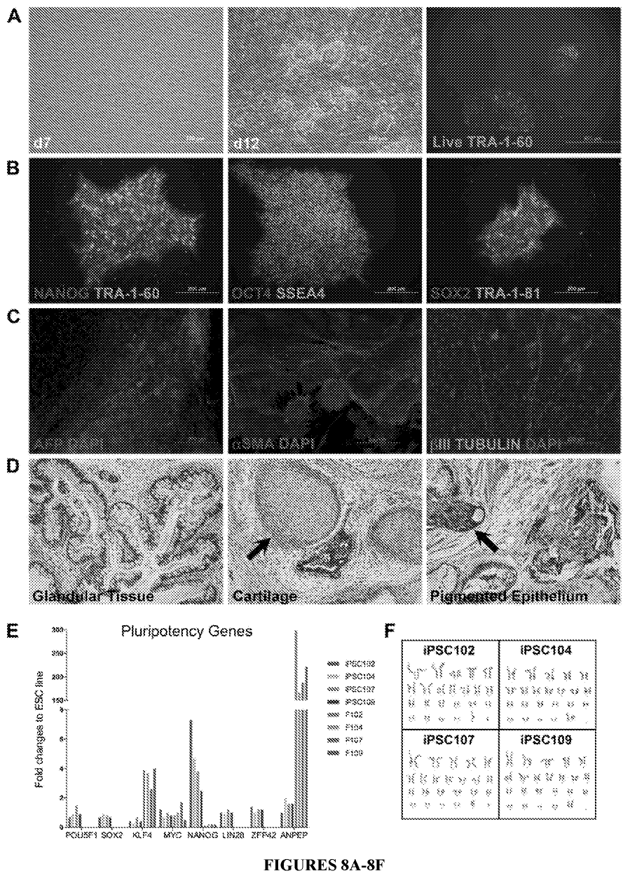

To show that the protocol provided herein can be applied to iPSC lines, skin biopsies were obtained from four PPMS patients. Fibroblast cultures were established from the biopsies, and iPSCs were generated using daily transfections with a cocktail of modified mRNAs, together with a cluster of miRNAs to improve the reprogramming efficiency for the most refractory lines (Stemgent). From day 12 to day 15 of reprogramming, TRA-1-60⁺ colonies (FIG. 8A) were identified by live staining, picked, expanded, and characterized by immunofluorescence for pluripotency markers (FIG. 8B).

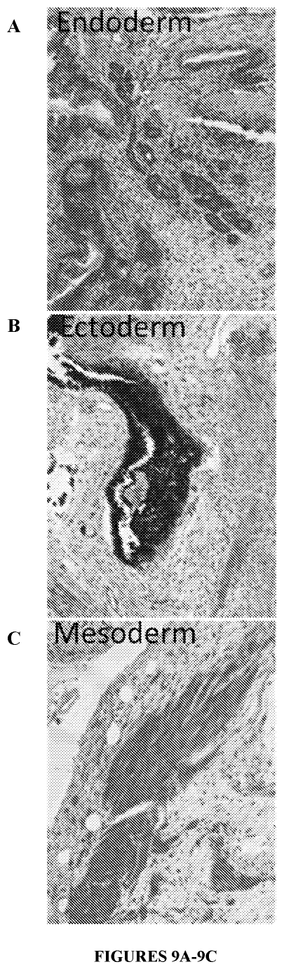

Expression profiling for seven pluripotency genes confirmed that all four iPSC lines exhibited a profile comparable to a reference hESC line and divergent from the parental fibroblasts (FIG. 8C). All iPSC lines displayed a normal karyotype (FIG. 8D) and were able to differentiate into cell types of the three germ layers, both in vitro, via spontaneous embryoid body differentiation (FIG. 8E), and in vivo via teratoma assay (FIG. 8F; FIG. 9).

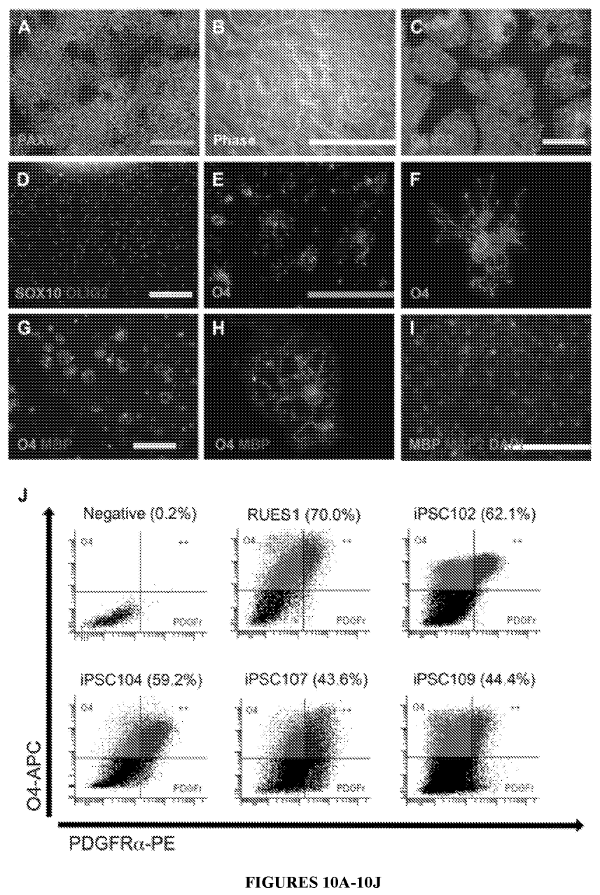

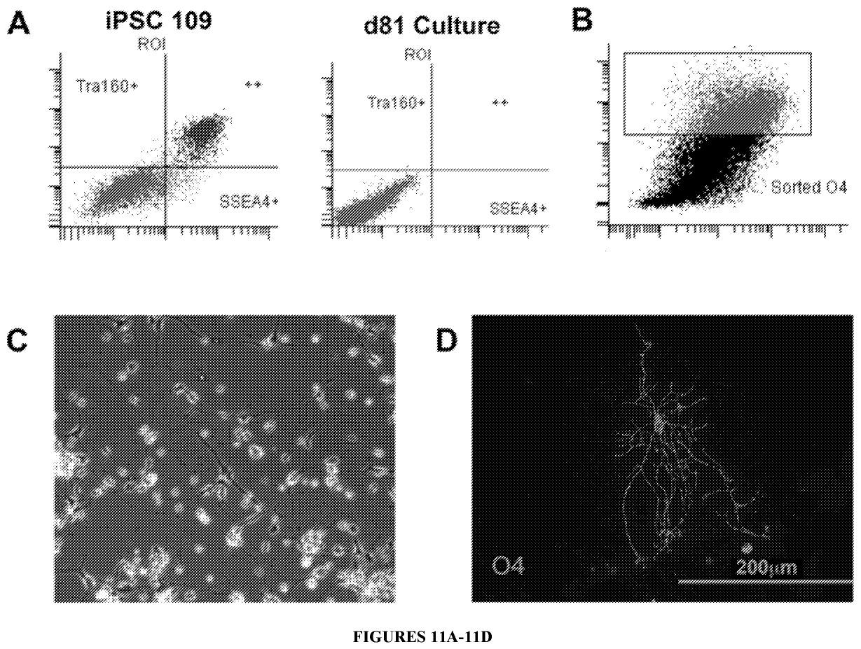

Next, whether the protocol was reproducible with PPMS-iPSC lines was assessed. All iPSC lines tested were found to perform similarly to the RUES1 line (FIG. 10A-10I). The protocol was greatly reproducible and highly efficient, as calculated by the frequency of sorted O4⁺ OPCs, with up to 70% O4⁺ cells from RUES1, and 43.6%-62.1% from the PPMS iPSC lines. Additionally, the O4⁺ fraction was found to contain a subpopulation of cells double positive with PDGFRα (FIG. 10J). O4⁺ cells could be easily purified by FACS, frozen and thawed, without losing their morphology (FIG. 11D).

### Example 3

### Axon Myelinate Mouse Brain by PPMS-Derived Late OPCs

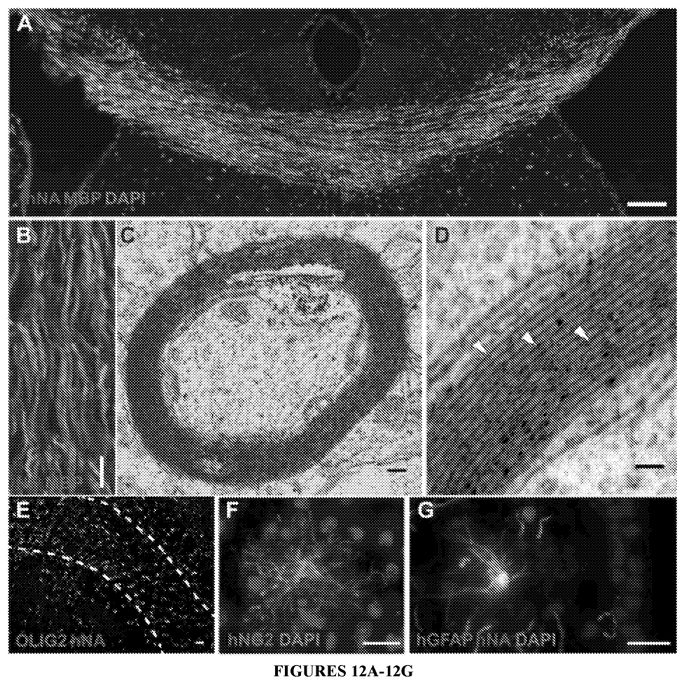

To verify that OPCs obtained through protocols provided herein were functionally myelinogenic, day 75 FACS-purified O4⁺ cells (10⁵ cells/animal) were injected into the forebrain of neonatal, immunocompromised shiverer mice (FIG. 11A). The injected cells were depleted of any contaminant iPSCs, as shown by flow cytometry analysis of pluripotency markers SSEA4 and TRA-1-60 (FIG. 11B). However, cultures were purified before in vivo transplantation to retain the potential for translation to clinical studies. Cells were frozen, thawed, and allowed to recover for 24-48 hours before transplantation (FIG. 11C). Animals were sacrificed at 12-16 weeks, at which point human hNA⁺ cells were distributed throughout the corpus callosum and forebrain white matter. The density of hNA⁺ cells in the corpus callosum at 12 weeks was 34,400 f 3,090 cells/mm³, and by 16 weeks, the number of human cells had approximately doubled since 12 weeks. The presence of cell clusters or overt tumorigenesis was not observed, and the proliferative fraction of engrafted hNA⁺ cells was 17% at 12 weeks, and decreased to only 8% Ki67⁺ at 16 weeks when only 5% of cells were PCNA⁺. Importantly, more than 80% of hNA⁺ cells in the corpus callosum co-expressed OLIG2 protein, suggesting that the engrafted cells were restricted to the oligodendrocyte lineage (FIG. 12E). Furthermore, human MBP⁺ oligodendrocytes were found diffusely throughout engrafted corpus callosum at 12 and 16 weeks (FIG. 12A). At 16 weeks, 31±3% of host mouse axons were ensheathed within the engrafted mouse corpus callosum (FIG. 12B).

Transmission electron microscopy on 16-week-old corpus callosum revealed mature compact myelin with the presence of alternating major dense and intraperiod lines (FIG. 12C, 12D); while uninjected shiverer/rag2 mice possessed thin and loosely wrapped myelin. Likewise, the thickness of myelin ensheathment, as assessed by g-ratio measurement, reflected a restoration of normal myelin in several callosal axons.

At 12 weeks, transplanted hNA⁺ cells remained as NG2⁺ OPCs in the corpus callosum (FIG. 12E), and by 16 weeks they started to migrate to the overlying cerebral cortex (FIG. 12F). Very few O4-sorted cells underwent differentiation as hGFAP⁺ astrocytes, and the majority of hGFAP⁺ cells were localized to the SVZ and around the ventricles (FIG. 12G), suggesting that the local environment may induce astrocytic differentiation in these regions. Similarly, hNESTIN-expressing cells were rarely found in corpus callosum and likewise concentrated in SVZ. Importantly, βIII-Tubulin⁺ neurons were not detected in any of the engrafted animals. Taken together, these data demonstrate that PPMS-derived O4-sorted cells were capable of mature oligodendrocyte differentiation in vivo and the formation of dense compact myelin resembling normal myelin in the brain.

### Example 4

### Detailed Experimental Procedures for Examples 1-3

Cell Lines

Three hESC lines and 4 hiPSC lines were used. RUES1 and HUES 45 are both NIH-approved hESC lines; OLIG2-GFP reporter line is derived from BG01 hESC line (University of Texas Health Science Center at Houston). Four iPSC lines were derived from skin biopsies of PPMS patients through the mRNA/miRNA method (Stemgent).

hPSC Culture Conditions

hESC and hiPSC lines were cultured and expanded with HUESM (Human Embryonic Stem Medium) medium and 10 ng/ml bFGF (Stemcell Technologies) onto mouse embryonic fibroblast (MEF) layer. For oligodendrocyte differentiation, cells were adapted to cultures onto Matrigel™-coated dishes and mTeSR1 medium (Stemcell Technologies). HUESM is composed by Knockout-DMEM, 20% Knock-out serum, glutamax 2 mM, NEAA 0.1 mM, 1×P/S and P-mercaptoethanol 0.1 mM, all purchased from Life Technologies (Grand Island, N.Y.). At all stages of differentiation cells are cultured in 5% CO₂ incubators.

Detailed Differentiation Protocol

PSCs were plated on Matrigel™ (BD Biosciences; San Jose, Calif.) at a density of 10×10³ cells/cm² in mTeSR1 medium (Stemcell Technologies; Vancouver, BC, Canada) containing 10 μM ROCK inhibitor, Y-27632 (Stemgent; Cambridge, Mass.) for 24 hours. This density of plated hPSCs was optimized to give a confluent well by day 8 and multilayered structures at day 12 of differentiation. This set up does not require significant PSC expansion, as only one well (80% confluent) of a 6-well plate contains enough cells to differentiate and isolate at least 2×10⁶ oligodendrocytes. Cells were incubated for 1-2 days, until hPSC colonies reached a diameter of 100-250 μm.

At the day of differentiation induction (day 0), medium was switched to Neural Induction Medium, which is mTeSR Custom Medium (Stemcell Technologies) containing the small molecules SB431542 10 μM (Stemgent) and LDN193189 250 nM (Stemgent), as well as 100 nM all-trans-RA (Sigma-Aldrich; St. Louis, Mo.). mTeSR Custom Medium has the same composition as the commercially available mTeSR-1 medium but without five factors that sustain pluripotency, namely lithium chloride, GABA, pipecolic acid, bFGF, and TGFβ1 (Stemcell Technologies). Instead of mTeSR Custom Medium, DMEM/F12 with the addition of about 25 μg/ml insulin could also be used. Media changes were performed daily until day 8, with fresh RA, SB431542, and LDN193189 added to the medium every day.

By day 8 cells should be confluent and PAX6 expression should be at its peak (FIG. 7A). At day 8, the medium was switched to N₂ Medium containing 100 nM RA, 1 μM SAG (EMD Millipore; Billerica, Mass.) or 100 ng/ml rhSHH (R&D Systems; Minneapolis, Minn.), changed daily, with fresh RA and SAG added to the medium every day.

By day 12, overconfluent cells were piling up and 3D structures were clearly visible, (FIG. 7B); this is an important checkpoint before proceeding with the differentiation. Cells expressed OLIG2 and NKX2.2 (FIG. 7C). At day 12, adherent cells were detached mechanically or enzymatically to allow for sphere formation. Sphere-formation enriched for the OLIG2⁺ progenitors. Only the OLIG2⁺ cells aggregated into spheres, whereas the OLIG2⁻ cells remained as single cells. The highest number of spheres was obtained, and ultimately of O4⁺ cells, using mechanical dispersion of the monolayer. More uniform spheres were obtained, in lower numbers, using enzymatic dispersion of the monolayer. For mechanical detachment, cells were detached using a cell lifter, breaking the monolayer of cells into small clumps so that nutrients could reach all the cells within the aggregate. Wells were inspected under the microscope to ensure that no cells were left attached. For enzymatic digestion, Accutase™ (1 ml/2 ml DMEM/F12 medium) was added to wells to dissociate the culture into a single-cell suspension.

Aggregates were re-plated into Ultra-low attachment plates in N₂B27 Medium containing 1 μM SAG, changing it every other day. At day 20, medium was switched to PDGF Medium, and 2/3 media changes were performed every other day. During media changes, gentle pipetting was used to break apart any aggregates sticking to one another. At day 30, spheres were plated onto plates coated with poly-L-ornithine hydrobromide (50 μg/ml; Sigma-Aldrich) and Laminin (20 μg/ml; Life Technologies) at a density of 2 spheres/cm² (about 20 spheres per well in a 6-well plate). This density was optimized to allow cells to migrate out from the sphere, proliferate and spread to the entire dish by the end of the protocol without the need for passaging. A p200 pipette was used to pick aggregates that were round, golden/brown with a dark center, having a diameter between 300 and 800 μm (FIG. 7D). Spheres that were completely transparent were avoided, as these do not differentiate to oligodendrocytes.

At this stage, plated spheres were cultured in a medium containing mitogen (Option A) or in a medium without any mitogen (Option B). Option A was optimized to obtain the highest yield of O4⁺ cells, while Option B was developed to provide a shorter and less costly version of the protocol.

Option A

Spheres were plated on pO/L plates, as described above, in PDGF Medium at day 30, changing 2/3 of the PDGF Medium every other day until day 75 of differentiation. The appearance of O4⁺ cells was assessed by live O4 staining from day 55 onwards (FIG. 6). At day 75, O4+ OPCs could be isolated by FACS (FIG. 7I). Alternatively, for terminal oligodendrocyte differentiation (FIG. 7G, 7H), cells were cultured in Glial Medium from day 75, changing 2/3 of the medium every 3 days for two weeks.

Option B

Spheres were plated on pO/L plates, as described above, in Glial Medium at day 30, changing 2/3 of the Glial Medium every other day until day 55 of differentiation. At day 55, O4⁺ cells were visualized by live O4 staining (FIG. 6A-F, FIG. 7F) or isolated by FACS (FIG. 6G). At day 75, O4+ OPCs could be isolated by FACS. Alternatively, cultures were kept in Glial Medium until day 75 to increase the efficiency of O4⁺ cells (FIG. 6). MBP+ cells were observed beginning at about day 60.

For both Option A and Option B protocols, aggregates at day 30, and cells at the end of the differentiation could be cryopreserved with a viability >70%. Aggregates' viability is based on the number of thawed spheres that re-attach onto pO/L coated dishes after thawing. The sorted O4⁺ cells could be frozen immediately after sorting. The expected post-thaw viability of the sorted O4⁺ cells is 70-80%.

Table 2 provides a list of media compositions used in the protocol.

TABLE 2
Detailed composition of culture media.

| Media | Components | Provider | Final Conc. | |
|---|---|---|---|---|
| N₂ Medium | DMEM/F12 | Life Technologies | 1X | |
| | Glutamax (100X) | Life Technologies | 1X | |
| | Non-Essential | Life Technologies | 1X | |
| | Amino Acids (100X) | | | |
| | β-Mercaptoethanol | Life Technologies | 1X | |
| | (1000X) | | | |
| | Penicillin- | Life Technologies | 1X | |
| | Streptomycin (100X) | | | |
| | N₂ Supplement (100X) | Life Technologies | | |
| N₂B₂₇ | N₂ Medium | Life Technologies | 1X | |
| | B₂₇ Supplement (50X) | | | |
| PDGF Medium | N₂B₂₇ Medium | R&D Systems | 10 | ng/ml |
| | PDGF | R&D Systems | 10 | ng/ml |
| | IGF-1 | R&D Systems | 5 | ng/ml |
| | HGF | EMD Millipore | 10 | ng/ml |
| | NT3 | Sigma-Aldrich | 25 | μg/ml |
| | Insulin | Sigma-Aldrich | 100 | ng/ml |
| | Biotin | Sigma-Aldrich | 1 | μM |
| | cAMP | Sigma-Aldrich | 60 | ng/ml |
| | T3 | | | |
| Glial Medium | N₂B₂₇ Medium | Sigma-Aldrich | 20 | μg/ml |
| | Ascorbic Acid | Sigma-Aldrich | 10 | mM |
| | HEPES | Sigma-Aldrich | 25 | μg/ml |
| | Insulin | Sigma-Aldrich | 100 | ng/ml |
| | Biotin | Sigma-Aldrich | 1 | μM |
| | cAMP | Sigma-Aldrich | 60 | ng/ml |
| | T3 | | | |

Derivation of Skin Fibroblasts from Punch Biopsies

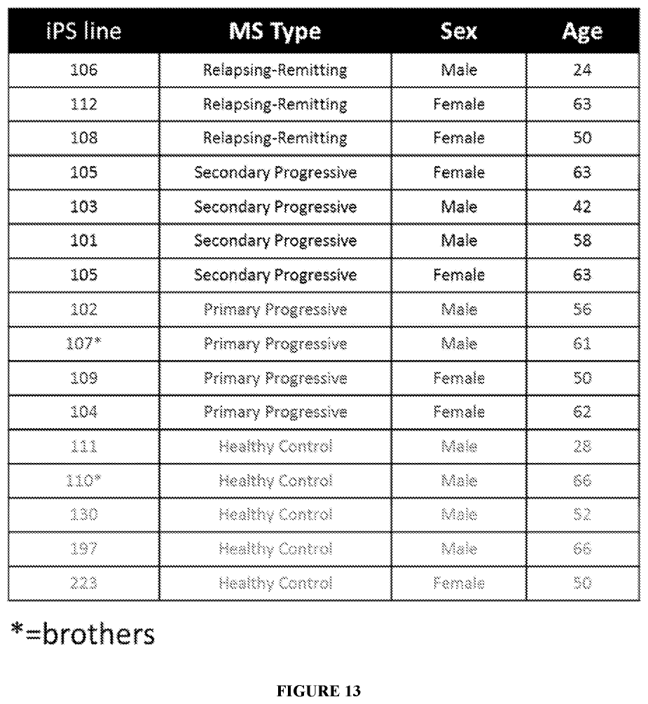

Skin biopsies were obtained from MS patients and healthy individuals (FIG. 13). Four de-identified patients at the Tisch Multiple Sclerosis Research Center of New York were diagnosed with PPMS according to the standard diagnostic criteria. Their biopsies were obtained upon institutional review board approval (BRANY) and informed consent. All patients are Caucasian. Patients 102 and 107 are male. 56 and 61 years old respectively; patients 104 and 109 are female, 62 and 50 years old respectively.

Skin biopsies of 3 mm were collected in Biopsy Collection Medium, consisting of RPMI 1460 (Life Technologies) and 1× Antibiotic-Antimycotic (Life Technologies). Biopsies were sliced into smaller pieces (<1 mm) and plated onto a TC-treated 35 mm dish for 5 minutes to dry and finally they were incubated in Biopsy Plating Medium, composed by Knockout DMEM, 2 mM GLUTAMAX™, 0.1 mM NEAA, 0.1 mM β-Mercaptoethanol, 10% Fetal Bovine Serum (FBS), 1× Penicillin-Streptomycin (P/S; all from Life Technologies) and 1% Nucleosides (EMD Millipore), for 5 days or until the first fibroblasts grew out of the biopsy. Alternatively, biopsies were digested with 1000U/ml Collagenase 1A (Sigma-Aldrich) for 1.5 hours at 37° C., washed, collected and plated onto 1% gelatin-coated 35 mm dish in Biopsy Plating Medium for 5 days. Fibroblasts were then expanded in Culture Medium, consisting of DMEM (Life Technologies), 2 mM GLUTAMAX™, 0.1 mM NEAA, 0.1 mM β-Mercaptoethanol, 10% FBS and 1×P/S changing medium every other day.

Reprogramming of Skin Fibroblasts

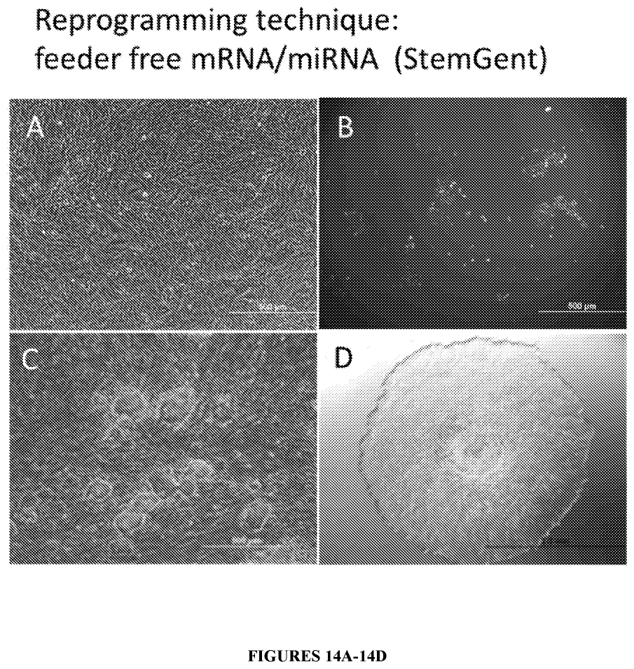

Skin fibroblasts at passage 3 to 5 were reprogrammed using the Stemgent mRNA/miRNA kit, which results in the generation of integration-free, virus free human iPSCs, through modified RNAs for OCT4, SOX2, KLF4, cMYC and LIN28 (FIG. 14). The addition of a specific cluster of miRNA has been found to increase the efficiency of reprogramming (Stemgent). Briefly, fibroblasts were plated onto Matrigel™-coated 6-well or 12-well plates in a 5.5×10³ cells/cm² density in culture medium. The following day, medium was replaced with NuFF-conditioned Pluriton reprogramming medium containing B18R. Cells were transfected for 11 consecutive days using STEMFECT™ as following: day 0 miRNA only, day 1 to day 3 mRNA cocktail only, d4 miRNA plus mRNA cocktail, day 5 to day 11 mRNA cocktail only. After day 11, visible colonies positively stained for live TRA-1-60 were picked and re-plated on MEFs with HUESM medium.

Teratoma Assay

Experiments were performed according to a protocol approved by the Columbia Institutional Animal Care and Use Committee (IACUC).

iPSC colonies were dissociated using Collagenase (Sigma-Aldrich) for 15 minutes at 37° C., washed, collected, and re-suspended in 200 μl HUESM. Cells were then mixed with 200 μl Matrigel™ (BD Biosciences) on ice, and were injected subcutaneously into immunodeficient mice (Jackson Laboratory; Bar Harbor, Me.). Teratomas were allowed to grow for 9-12 weeks, isolated by dissection, and fixed in 4% PFA overnight at 4° C. Fixed tissues were embedded in paraffin, sectioned at 10 μm thickness, and stained with hematoxylin and eosin (H&E).

Spontaneous Differentiation In Vitro

iPSCs were dissociated with Accutase™ (Life Technologies) for 5 minutes at 37° C. and seeded into Ultra-Low attachment 6-well plates in HUESM without bFGF, changing media every other day. After 3 weeks of culture, embryoid bodies (EBs) were plated onto 1% gelatin-coated TC-treated dishes for another 2 weeks. EBs and their outgrowth were fixed in 4% PFA for 8 minutes at RT and immunostained for the appropriate markers.

RNA Isolation and qRT-PCR

RNA isolation was performed using the RNeasy Plus Mini Kit with QIAshredder (Qiagen; Hilden, Germany). Briefly, cells were pelleted, washed with PBS, and re-suspended in lysis buffer. Samples were then stored at −80° C. until processed further according to manufacturer's instructions. RNA was eluted in 30 μl RNase free ddH₂O and quantified with a NanoDrop 8000 spectrophotometer (Thermo Scientific; Somerset, N.J.).

For qRT-PCR, cDNA was synthesized using the GoScript™ Reverse Transcription System (Promega; Madison, Wis.) with 0.5 μg of RNA and random primers. 20 ng of cDNA were then loaded to a 96-well reaction plate together with 10 μl GoTaq® qPCR Master Mix and 1 μl of each primer (10 nM) in a 20 μl reaction and the plate was ran in Stratagene Mx300P qPCR System (Agilent Technologies; Santa Clara, Calif.). Table 3 lists primer sequences.

TABLE 3
Sequences of Primers Used for qRT-PCR

| SEQ | | | SEQ | |
|---|---|---|---|---|
| ID. | Target | | ID. | |
| NO. | gene | Forward primer | NO. | Reverse primer |
| 1 | PAX6 | TTTGCCCGAGAAAG | 2 | CATTTGGCCCTTC |
| | | ACTAGC | | GATTAGA |
| 3 | OLIG2 | TGCGCAAGCTTTCC | 4 | CAGCGAGTTGGT |
| | | AAGA T | | GAGCATGA |
| 5 | NKX2.2 | GACAACTGGTGGCA | 6 | AGCCACAAAGAAAG |
| | | GATTTCGCTT | | GAGTTGGACC |
| 7 | PTCH1 | ATCTGCACCGGCCC | 8 | CCACCGCGAAGGCC |
| | | AGCTACT | | CCAAATA |
| 9 | SHH | AAACACCGGAGCGG | 10 | GGTCGCGGTCAGAC |
| | | ACAGGC | | GTGGTG |

Nanostring Analysis for Pluripotency

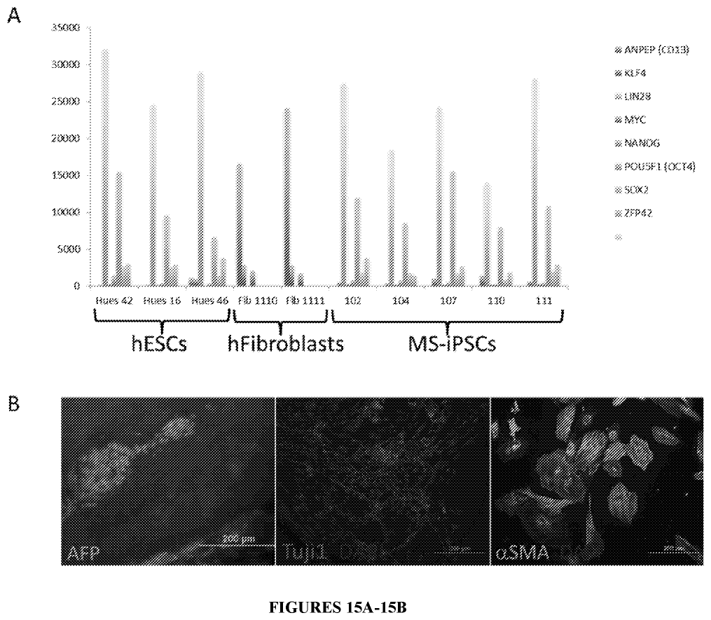

RNA was isolated from undifferentiated iPSCs and hESC HUES45 as previously described. RNA (100 ng/sample) was loaded for the hybridization with the specific Reporter Code Set and Capture Probe Set (NanoString Technologies; Seattle, Wash.) according to manufacturer's instructions. Data were normalized to the following housekeeping genes: ACTB, POLR2A, ALAS1. Data were expressed as fold changes to the expression of the hESC line (HUES45=1). See FIG. 15.

Karyotyping

All iPSC-lines were subjected to cytogenetic analysis by Cell Line Genetics to confirm a normal karyotype.

Immunostaining and Imaging

Cells were washed 3× in PBS-T (PBS containing 0.1% Triton-X100) for 10 minutes, incubated for 2 hours in blocking serum (PBS-T with 5% goat or donkey serum) and primary antibodies were applied overnight at 4° C. (Table 4). The next day, cells were washed 3× in PBS-T for 15 minutes, incubated with secondary antibodies for 2 hours at room-temperature (RT), washed 3× for 10 minutes in PBS-T, counterstained with DAPI for 15 minutes at RT and washed 2× in PBS. Invitrogen™ Alexa Fluor secondary antibodies, goat or donkey anti-mouse, rat, rabbit, goat and chicken 488, 555, 568, and 647 were used at 1:500 dilution (Life Technologies).

Images were acquired using an Olympus™ IX71 inverted microscope, equipped with Olympus DP30BW black and white digital camera for fluorescence and DP72 digital color camera for H&E staining. Fluorescent colors were digitally applied using the Olympus software DP Manager or with ImageJ™. For counting, at least three non-overlapping fields were imported to ImageJ®, thresholded and scored manually.

Flow Cytometry

Cells were enzymatically harvested by Accutase™ treatment for 25 minutes at 37° C. to obtain a single cell suspension. Cells were then re-suspended in 100 μl of their respective medium containing the appropriate amount of either primary antibody or fluorescence-conjugated antibodies and were incubated on ice for 30 minutes shielded from light. When secondary antibodies were used, primary antibodies were washed with PBS and secondary antibodies were applied for 30 minutes on ice. Stained or GFP expressing cells were washed with PBS and sorted immediately on a 5 laser BD Biosciences ARIA-IIu™ Cell Sorter using the 100 μm ceramic nozzle, and 20 psi. DAPI was used for dead cell exclusion. Flow cytometry data were analyzed using BD FACSDiva™ software.

Transplantation into Shiverer (Shi/Shi)×Rag2^{-/-} Mice.

All experiments using shiverer/rag2 mice (University of Rochester; Windrem, M. S. el al., Cell Stem Cell. 2:553-565 (2008)) were performed according to protocols approved by the University at Buffalo Institutional Animal Care and Use Committee (IACUC). FACS-sorted O4⁺ OPCs that had been previously cryopreserved were thawed and allowed to recover for 1-2 days prior to surgery by plating on pO/L dishes in PDGF Medium. Cells were prepared for injection by re-suspending cells at 1×10⁵ cells per μl.

Injections were performed as previously described. Sim, F. J. et al. (2011). Pups were anesthetized using hypothermia and 5×10⁴ cells were injected in each site, bilaterally at a depth of 1.1 mm into the corpus callosum of postnatal day 2-3 pups. Cells were injected through pulled glass pipettes, inserted directly through the skull into the presumptive target sites. Animals were sacrificed and perfused with saline followed by 4% paraformaldehyde at 12-16 weeks. Cryopreserved coronal sections of mouse forebrain (16 μm) were cut and sampled every 160 μm. Sim, F. J. et al. (2011). Human cells were identified with mouse antihuman nuclei (hNA) and myelin basic protein-expressing oligodendrocytes were labeled with MBP. Human astrocytes and OPCs were stained with human-specific antibodies against hGFAP and hNG2 respectively. Mouse neurofilament (NF) was stained by 1:1 mixture of SMI311 and SMI312. Invitrogen™ Alexa Fluor secondary antibodies, goat anti-mouse 488, 594, and 647 were used at 1:500 dilution (Life Technologies). For transmission electron microscopy, tissue was processed as described previously. Sim, F. J. et al., Molec. Cell. Neurosci. 20:669-682 (2002). Table 4 provides a list of primary antibodies used.

TABLE 4
Primary Antibodies

| Antigen | Dil. | Host | Provider |
|---|---|---|---|
| OCT4 | 1:250 | Rabbit | Stemgent |
| TRA-1-60 | 1:250 | Mouse | Millipore |
| SOX2 | 1:250 | Rabbit | Stemgent |
| TRA-1-81 | 1:250 | Mouse | Millipore |
| NANOG | 1:100 | Rabbit | Cell Signal. |
| SSEA4 | 1:250 | Mouse | Abcam |
| AFP | 1:300 | Rabbit | Dako |
| αSMA | 1:300 | Mouse | Sigma |
| βIII-Tubulin | 1:500 | Chicken | Neuromics |
| PAX6 | 1:250 | Rabbit | Covance |
| OLIG2 | 1:500 | Rabbit | Millipore |
| NKX2.2 | 1:75 | Mouse | DSHB |
| SOX10 | 1:100 | Goat | R&D Sys. |
| NG2 | 1:200 | Mouse | BD Biosci. |
| PDGFRα-PE | 1:5 | Mouse | BD Biosci. |
| O4 | 1:30 | Mouse | Goldman lab |
| MBP | 1:200 | Rat | Millipore |
| MAP2 | 1:5000 | Chicken | Abcam |
| GFAP | 1:750 | Rabbit | Dako |
| hNA clone 235-1 | 1:100 | Mouse | Millipore |
| GFAP (in vivo) | 1:800 | Mouse | Covance |
| NG2 (in vivo) | 1:800 | Mouse | Millipore |
| SMI311, SMI312 | 1:800 | Mouse | Covance |
| (mNeurofilament) | | | |

### Example 5

### A Screen for Astrocyte-Specific Surface Markers Identifies CD49f

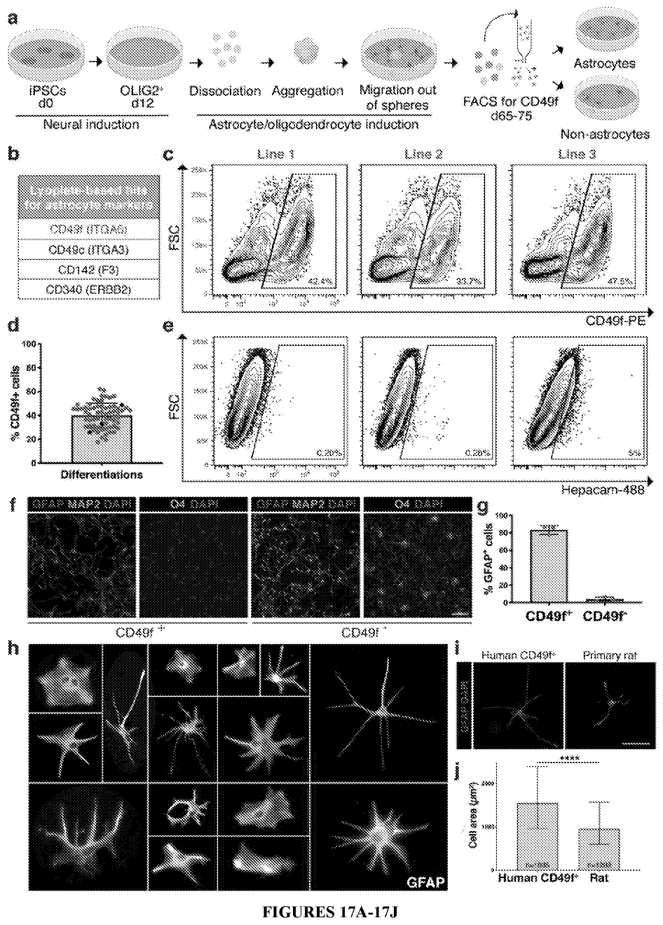

To enable the isolation of pure hiPSC-astrocytes, a screen of surface antigens was performed on a mix of neural cells derived from an oligodendrocyte protocol. This protocol generates OLIG2⁺ progenitors through retinoic acid and sonic hedgehog signaling, mimicking embryonic development in the spinal cord. When OLIG2⁺-enriched neural spheres are plated down, neurons, astrocytes and oligodendrocyte progenitor cells migrate out in order, and after day 65 immature oligodendrocytes can be purified using the O4 sulfate glycolipid antigen. An analogous sorting strategy to isolate astrocytes for functional studies was developed (FIG. 17a). To identify astrocyte-specific markers, day 78 cultures were digested into single cells and a panel of 242 antibodies to surface antigens within the O4⁺ and O4⁻ populations was tested. Sorted cells were re-plated, fixed and stained for glial fibrillary acidic protein (GFAP), an astrocyte-specific cytoplasm intermediate filament. Among the candidates that showed an enrichment of GFAP⁺ cells, 4 antigens whose mRNA expression was reported to be higher in astrocytes than in other CNS cell types were identified (FIG. 17b). CD49f was then selected for further validation using three independent iPSC lines from healthy subjects, from which an average of 40% CD49f⁺ cells were isolated (FIG. 17c, 17d, 25a, 25b). Of note, HepaCAM was minimally expressed by hiPSC-derived cells at this stage (FIG. 17e, 25c, 25d), emphasizing the need for an alternative marker. GFAP quantification in the FACS-sorted fractions revealed an average of 83% GFAP⁺ cells in the CD49f⁺ versus 3% in the CD49f⁻ population, which was enriched in MAP2⁺ cells and O4⁺ oligodendrocytes (FIG. 17f,17g). Interestingly, CD49f⁺ astrocytes were highly heterogeneous in morphology, echoing the complexity and diversity of astrocytes reported in vivo (FIG. 17h). Consistent with previous studies of human astrocytes, CD49f astrocytes were also larger than primary rat astrocytes (FIG. 17i, 17j).

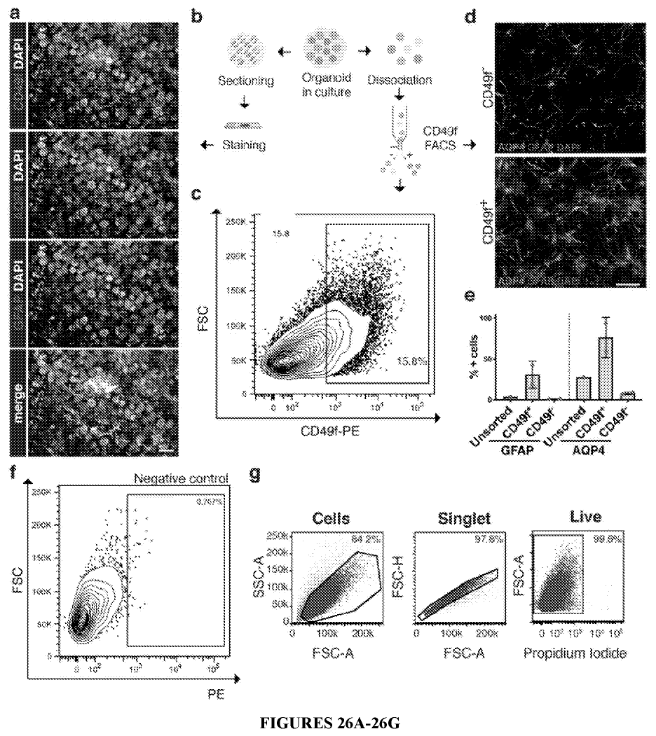

As CD49f is a laminin receptor, and the differentiation protocol described herein uses laminin coating, astrocytes from 3D cortical organoids cultured in the absence of laminin were analyzed. The organoid protocol generates iPSC-derived cortical astrocytes, whereas astrocyte protocols provided here are patterned towards spinal cord. Nevertheless, in sorted cells from digested cortical organoids, CD49f was still co-expressed with AQP4 and GFAP (FIG. 26), and astrocytes were enriched in the CD49f fraction, with an average of 31% GFAP⁺ cells and 77% AQP4⁺ cells, as opposed to 1% GFAP⁺ cells and 8% AQP4⁺ cells in the CD49f⁻ fraction (FIG. 26b-g). These findings confirm that CD49f expression is neither dependent on laminin coating nor spinal cord-specific.

### Example 6

### Transcriptome Profile of CD49f⁺ Astrocytes Reveals Typical Astrocyte Markers

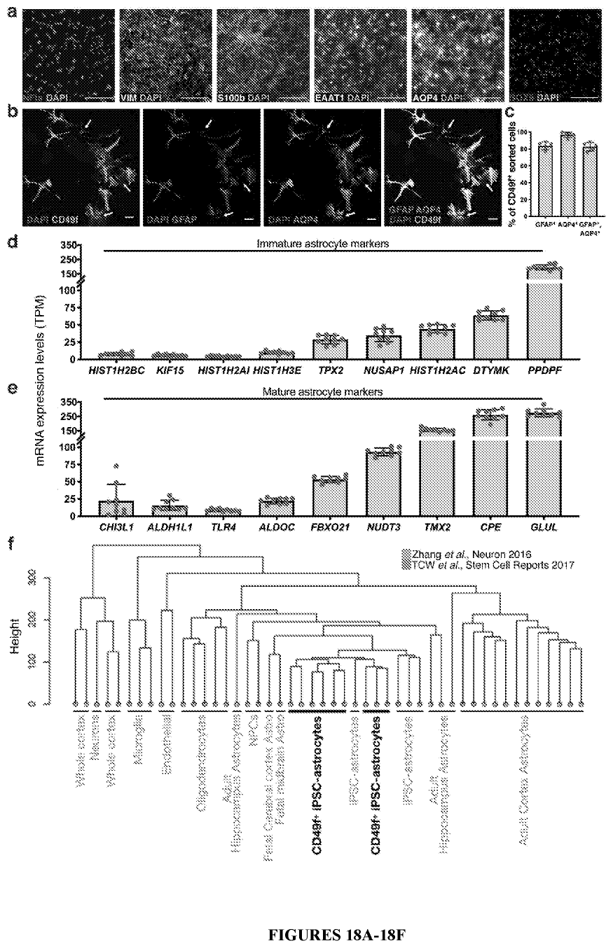

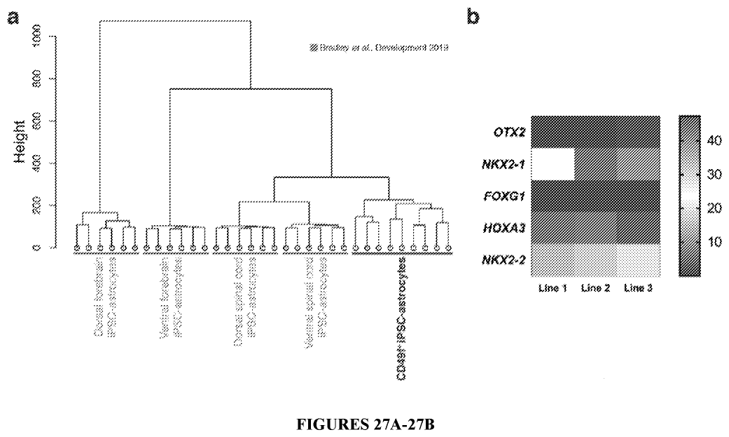

To verify the identity of CD49f⁺ cells, the presence of additional astrocyte makers was assessed. CD49f astrocytes stained positive for AQP4, SOX9, EAAT1, NFIa, VIM, and S100β (FIG. 18a). Interestingly, about 97% of the cells were AQP4⁺, but only 84% were GFAP⁺ (FIG. 18b, 18c), in line with findings that GFAP does not identify all CNS astrocytes and with the reported heterogeneity of GFAP levels in different brain regions. Transcriptomic analysis via RNA-Seq of CD49f⁺ cells revealed expression of several mature and immature astrocyte genes, with low inter-line variability (FIG. 18d, 18e). Hierarchical clustering of RNA-Seq data confirmed that hiPSC-astrocytes cluster close to fetal primary human astrocytes, and to hiPSC-astrocytes generated with an alternative differentiation protocol, but are distinct from neurons, oligodendrocytes, microglia, and endothelial cells (FIG. 18f). Spinal cord identity of the CD49f+ cells generated using the protocol provided herein was also confirmed, by performing hierarchical clustering of RNA-Seq data with a recent study of regionally specified hiPSC-astrocytes (FIG. 27a-b). Gene expression data has been made available in a user-friendly, searchable online database (nyscfseq.appspot.com/).

### Example 7

### Single-Cell RNA Transcriptome Analysis Demonstrates that CD49f Enriches for Mature hiPSC-Derived Astrocytes

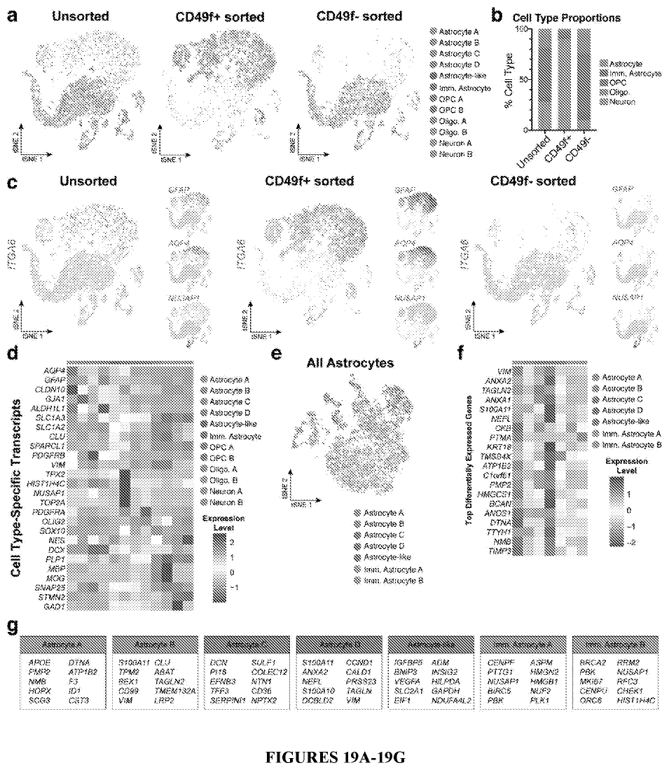

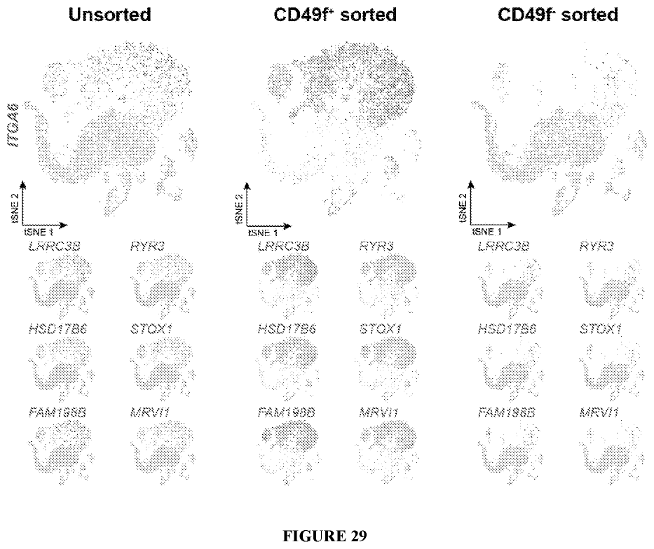

The expression of both mature and immature astrocyte markers in bulk transcriptome analysis of CD49f⁺ iPSC-astrocytes could reflect either the cells being in an intermediate state of maturation, or a mixture of immature and mature astrocytes. To resolve this question, single-cell RNA sequencing of unsorted cultures following differentiation as well as sorted CD49f⁺ astrocytes and CD49f⁺ cells was performed. Unsorted cultures contained the following subpopulations: mature astrocytes (28%; defined as GFAP⁺), immature astrocytes (5%; defined as NUSAP1+), oligodendrocyte progenitor cells (OPC, 48%), oligodendrocytes (13%), and neurons (6%). The sorted CD49f fraction was enriched for mature astrocytes (90%), while the sorted CD49f⁻ cells were depleted of mature astrocytes (10%) and enriched for all other cell types (FIG. 19a, 19b). ITGA6 expression primarily overlaps with that of mature markers GFAP and AQP4, but not immature marker NUSAP1 or OPC markers PDGFRA and CSPG4, confirming that sorting based on CD49f enriched for mature astrocytes (FIG. 19c, 19d, 28). Additionally, CD49f⁺ hiPSC-astrocytes expressed the human-specific astrocyte markers LRRC3B, HSSD17B6, FAM198B, RYR3, STOX1, and MRVI1 (FIG. 29).

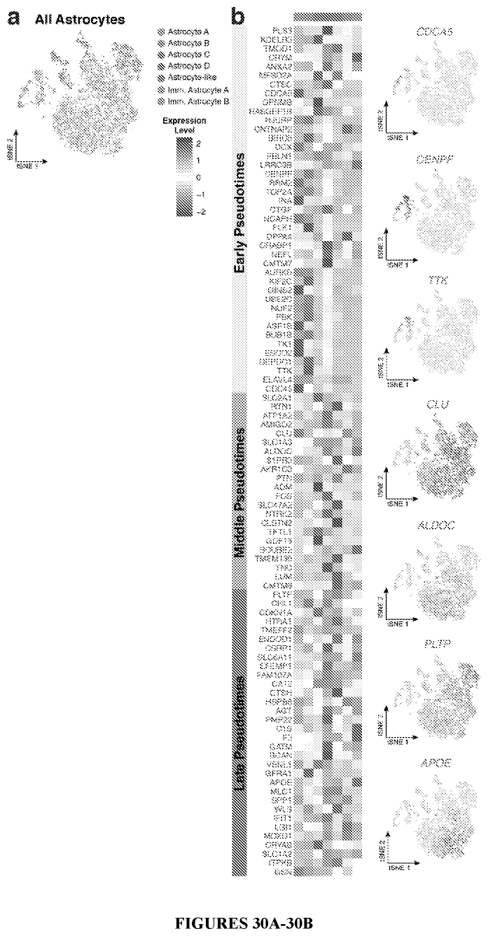

To further evaluate potential astrocyte heterogeneity indicated by the scRNA-seq analysis, only astrocytes defined by the initial clustering scheme were subsetted and reintegrated, and two immature astrocyte clusters, four mature astrocyte clusters, and one astrocyte-like cluster were subsequently identified (FIG. 19e, 19f). Immature astrocyte clusters, which are enriched in the CD49f⁻ fraction, had higher expression of early pseudotime transcripts, while mature astrocyte clusters, enriched in the CD49f⁺ fraction, had higher expression of middle and late pseudotime transcripts (FIG. 30).

### Example 8

### CD49f⁺ Astrocytes can be Isolated from Human Fetal Brains and are Present in Human Adult Brains

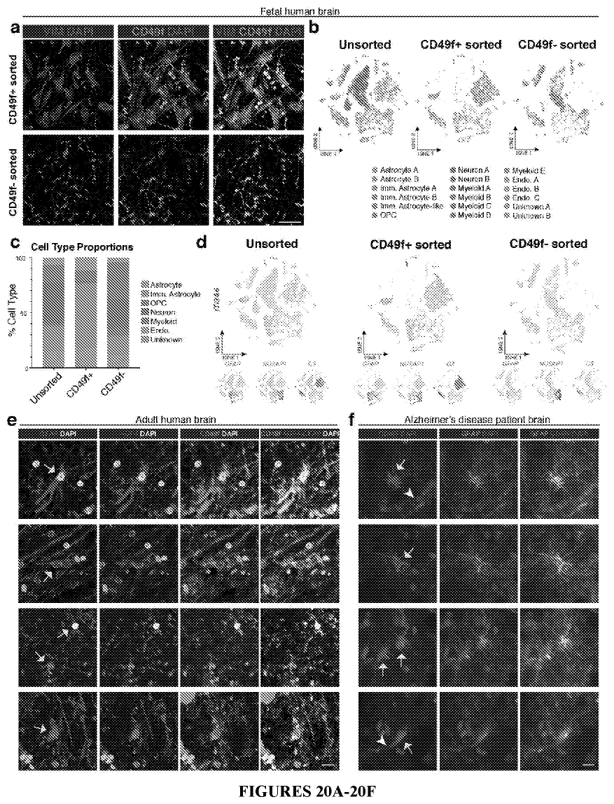

To assess whether CD49f can be used to isolate astrocytes from primary human tissues, cells from human fetal brain were dissociated and sorted, and the CD49f⁺ fraction was found to be highly enriched with vimentin astrocytes (FIG. 20a). Single-cell transcriptomic analysis showed that total digested fetal brain cells consisted of mature astrocytes, immature astrocytes, OPCs, neurons, myeloid cells, and endothelial cells (FIG. 20b). Sorting for CD49f resulted in an enrichment of astrocytes (from 37% to 76%) and endothelial cells (from 6% to 11%) (FIG. 20c). ITGA6 expression overlapped with expression of mature astrocyte marker GFAP and immature astrocyte marker C3 (FIG. 20d). Without being limited by theory, this indicates potential variation in ITGA6 expression across immature astrocyte populations, as C3⁺ immature astrocytes appear to have higher ITGA6 expression than NUSAP1⁺ immature astrocytes, which make up a large proportion of the immature astrocytes in the CD49f⁻ fraction of the sort (FIGS. 20b, 20d).

Whether CD49f is maintained in astrocytes from adult human brains was investigated next. Staining showed that CD49f co-localizes with AQP4 and GFAP in adult brain tissue sections from a healthy donor (FIG. 20e) as well as in an AD patient (FIG. 20f), corroborating CD49f as a novel marker for human astrocytes in vivo as well as in hiPSC cultures. Brain sections also showed CD49f⁺ endothelial cells that were not GFAP⁺ (FIG. 20f).

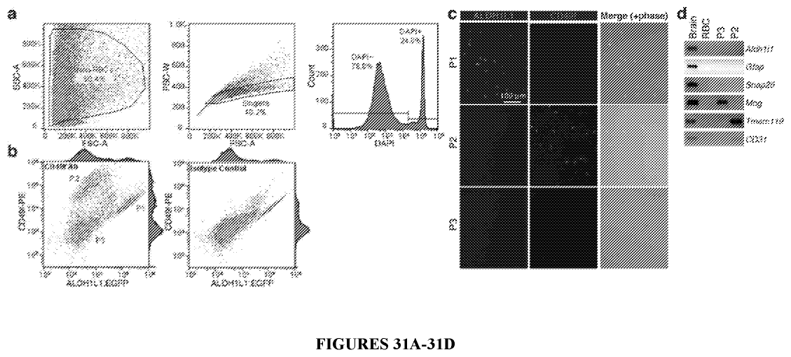

Interestingly, when CD49f isolation from whole mouse brain of Aldh1/1^{eGFP} mice was tested, no CD49f⁺ astrocytes were ALDH1L1⁺ (FIG. 31), suggesting that CD49f could be human-specific.

### Example 9

### CD49f⁺ Astrocytes Perform Physiological Astrocyte Functions In Vitro

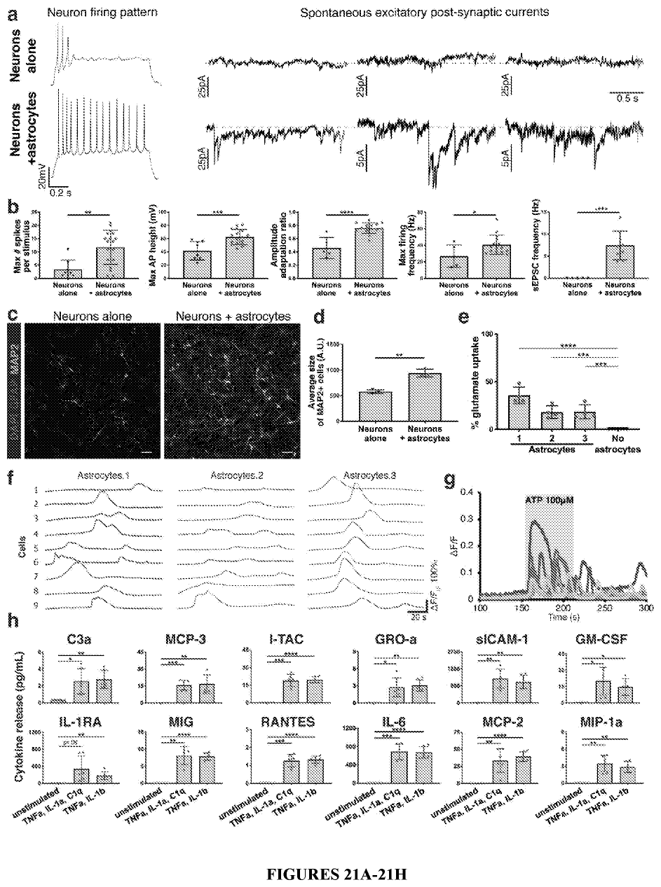

To assess the capacity of astrocytes to support neuronal function in vitro, co-cultures of CD49f⁺ hiPSC-astrocytes with hiPSC-derived neurons were set up. Neurons that were co-cultured with astrocytes for two weeks displayed more developed electrophysiological properties than neurons alone, including increases in number of action potentials per Is depolarizing stimulus, maximum firing frequency, maximum height of action potential, and amplitude adaptation ratio (FIGS. 21a, 21b). Furthermore, only neurons co-cultured with astrocytes exhibited spontaneous excitatory post synaptic currents (sEPSC) indicating advanced synaptogenesis (FIGS. 21a, 21b), as previously reported for adult human and rodent astrocytes. Neurons co-cultured with CD49f⁺ astrocytes had a larger MAP2 area than those cultured without astrocytes (FIGS. 21c, 21d), highlighting an increase in neurite length in these cells that also corroborates previous observations. Together, these data demonstrate that CD49f⁺ hiPSC-astrocytes are functional in supporting neurite outgrowth, neuronal function, and synapse formation.

Other important astrocyte physiological functions in vitro were tested next. CD49f⁺ hiPSC-astrocyte cultures efficiently take up glutamate, mimicking a crucial function in vivo for preventing neuronal glutamate excitotoxicity (FIG. 21e). Whether they would exhibit calcium transients was also investigated, as it is known that astrocytes have both spontaneous and inducible calcium signals that can be detected in vivo in different brain regions and in vitro. Cytosolic calcium levels were monitored and—like primary purified human astrocytes in vitro and mouse astrocytes visualized in vivo—spontaneous calcium transients were present in CD49f⁺ astrocytes from all lines (FIG. 21f). Furthermore, CD49f⁺ astrocytes robustly responded to 60s application of extracellular ATP at 100 μM (FIG. 21g).

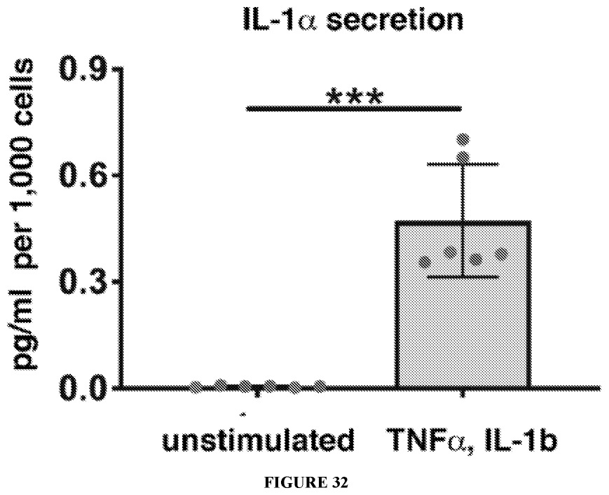

Astrocytes are immunocompetent cells, able to respond to inflammatory stimuli by releasing additional pro-inflammatory molecules. To test this in vitro, CD49f⁺ astrocytes were stimulated with either with IL-1β and TNFα or with TNFα, IL-1α, and C1q, which drives the neurotoxic A1 state reported in rodent cells. Pro-inflammatory cytokine secretion was significantly increased following both types of stimulation. In particular, IL-6 and soluble ICAM-1 showed the greatest fold changes compared to unstimulated cells, and notably, IL-la was secreted upon IL-1β and TNFα stimulation (FIG. 21h, FIG. 32).

### Example 10

### Human A1-Like Reactive Astrocytes Lose Functional Capacity for Phagocytosis and Glutamate Uptake

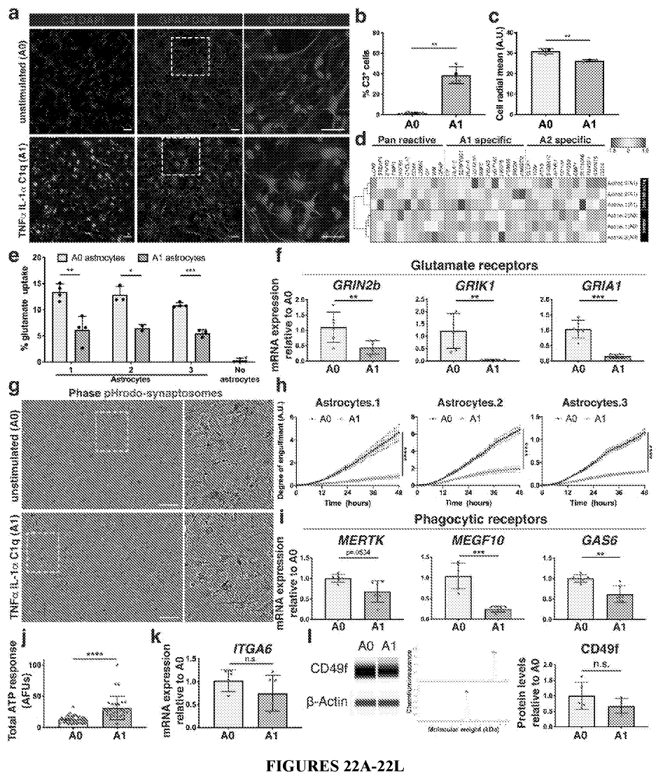

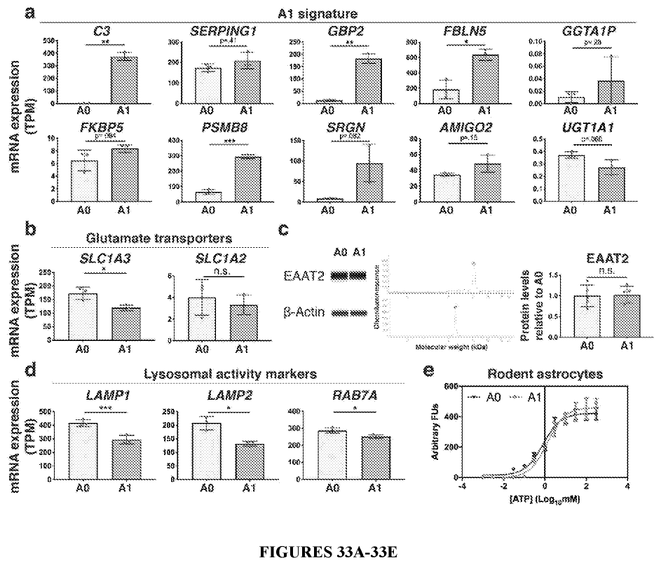

Studies have modeled inflammation-stimulated reactivity in hiPSC-derived astrocytes in vitro. The transcriptomic profile and functionality of CD49f⁺ hiPSC-astrocytes stimulated with TNFα, IL-1α, and C1q to drive an A1 reactive state was characterized next. Human iPSC-derived A1-like astrocytes were C3-positive like their rodent counterparts (FIGS. 22a, 22b), and they also exhibited the morphological changes observed in reactive astrocytes in vivo, losing finer processes and becoming hypertrophied (FIGS. 22a, 22c). To evaluate conservation of the defined rodent A1 signature in human cells, RNA-Seq on human stimulated (A1) vs. unstimulated (A0) astrocytes was performed (FIG. 22d), followed by assessment which transcripts were upregulated in reactive astrocytes in rodents, classified as pan-reactive, specifically induced by neuroinflammation (A1) or induced by ischemia (A2). Human A1-like cells also upregulated most rodent A1-specific genes (FIGS. 22d, 33). SLC1A3, encoding the glutamate transporter GLAST, was downregulated in A1-like astrocytes, while SLC1A2, encoding the glutamate transporter EAAT2 (GLT-1) was unchanged at both the RNA and protein level (FIG. 33b-c). Importantly, downregulation of glutamate receptors corresponded to impaired glutamate uptake in A1-like reactive cells (FIG. 22e-f). Moreover, the decrease in mRNA levels of phagocytic receptors MERTK and MEGF10 and bridging molecule GAS6 paralleled a significant decrease in phagocytosis of synaptosomes in hiPSC-derived A1-like reactive astrocytes (FIGS. 22g, 22h, 22i). This was accompanied by a decrease in expression of lysosomal markers LAMP1, LAMP2, and RAB7A (FIG. 33d). When assessing ATP response, hiPSC-derived A1-like astrocytes had a stronger response than A0 astrocytes (FIG. 22j), as opposed to rodent astrocytes, where ATP response does not change in the A1 state (FIG. 33e). Of note, hiPSC-derived A1-like astrocytes retained both ITGA6 expression and CD49f protein levels, verifying CD49f as a reactivity-independent marker (FIG. 22k-l). The full gene expression dataset from iPSC-astrocytes is available in the online database mentioned above. Taken together, characterization of human A1-like astrocytes, enabled by the technique for astrocyte isolation provided herein, establishes hiPSC-derived astrocytes as a powerful in vitro model to investigate molecular mechanisms linked to A1 pathogenesis.

### Example 11

### Human A1 Reactive Astrocytes are Neurotoxic

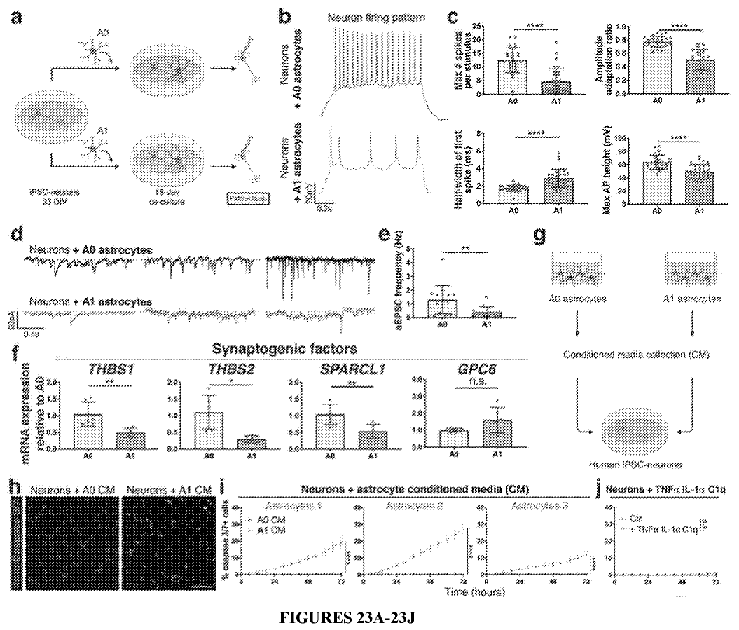

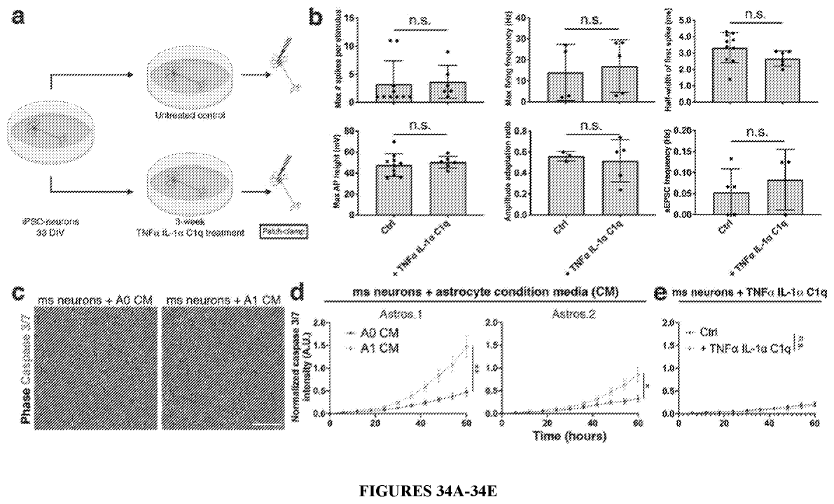

To evaluate the effects of hiPSC-derived A1-like astrocytes on neurons, unstimulated (A0) or A1-like astrocytes were co-cultured with hiPSC-neurons for 18 days and electrophysiological analysis was performed (FIG. 23a). Neurons co-cultured with A1-like astrocytes had a less mature firing pattern than neurons cultured with unstimulated astrocytes, including a lower number of spikes per stimulus, a larger spike half-width, a smaller amplitude adaptation ratio, and a smaller spike height (FIGS. 23b, 23c). Neurons co-cultured with A1-like astrocytes also had a smaller sEPSC frequency than neurons cultured with unstimulated astrocytes (FIGS. 23d, 23e). This is consistent with the significant downregulation of synaptogenic factors seen in A1 astrocytes, which could lead to dysfunctional astrocytes that are unable to support neuronal function and synapse formation, as previously shown in rodents (FIG. 23f). Direct treatment of hiPSC-neurons with TNFα, IL-1α, and C1q had no effect on neuronal maturation (FIG. 34a-b), supporting the hypothesis that the impaired maturation observed in neurons was astrocyte-mediated. To assess apoptosis, neurons were treated with astrocyte conditioned media collected from A0 and A1 astrocytes and measured caspase 3/7 levels. Both hiPSC-neurons and primary mouse neurons exhibited increased apoptosis following treatment with A1-like conditioned media, but not in response to direct treatment with TNFα, IL-1α, and C1q (FIG. 23g-j, FIG. 34c-e). These findings demonstrate the specific neurotoxicity of A1 hiPSC-derived astrocytes.

### Example 12

### Astrocyte Maturity Influences Response to A1 Stimulation

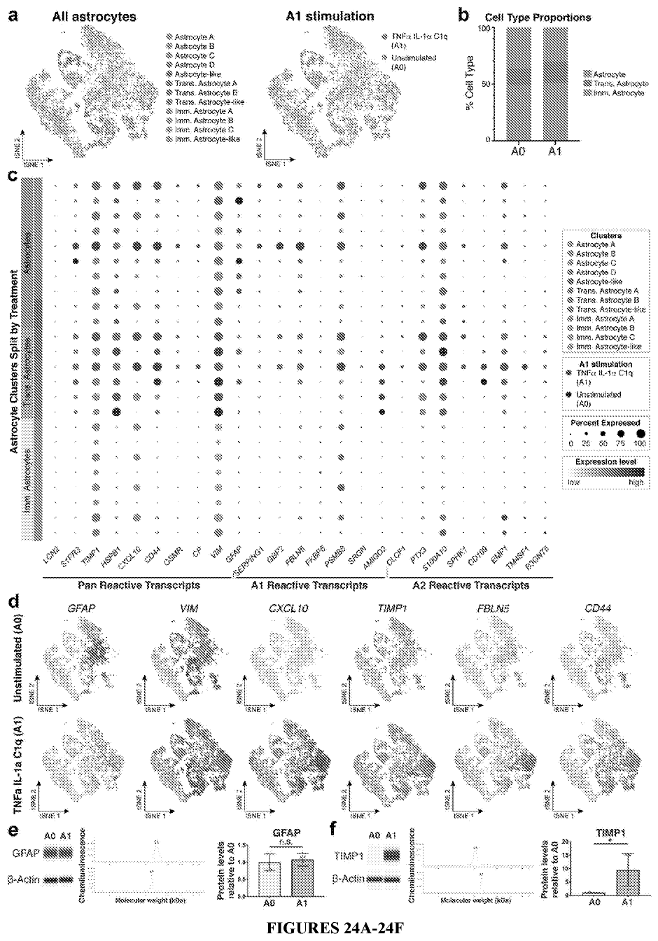

Given that unsorted cultures contain astrocytes at sequential stages of maturation (FIG. 19a), their response to TNFα, IL-1α, and C1q stimulation was assessed by performing single-cell RNA-Seq 24 hours after treatment with this inflammatory cocktail. After subsetting and reintegrating only astrocytes at all levels of maturation, 5 mature astrocyte clusters, 3 transitioning astrocyte clusters, and 4 immature astrocyte clusters were identified (FIG. 24a-b). Intriguingly, comparing single-cell data from A0 versus A1 indicated a maturation-dependent response to inflammatory stimuli, with mature astrocytes showing a greater response than immature astrocytes (FIG. 24c). Expression data also revealed CXCL10, TIMP1, FBLN5 and CD44 as better markers of reactivity than GFAP and VIM (FIG. 24d). This was confirmed at the protein level: GFAP levels were similar between A0 and A1 astrocytes (FIG. 24e), while TIMP1 levels were upregulated 9.5-fold in A1 astrocytes (FIG. 24f).

### Example 13

### Detailed Experimental Procedures for Examples 5-12

Human iPSC Lines

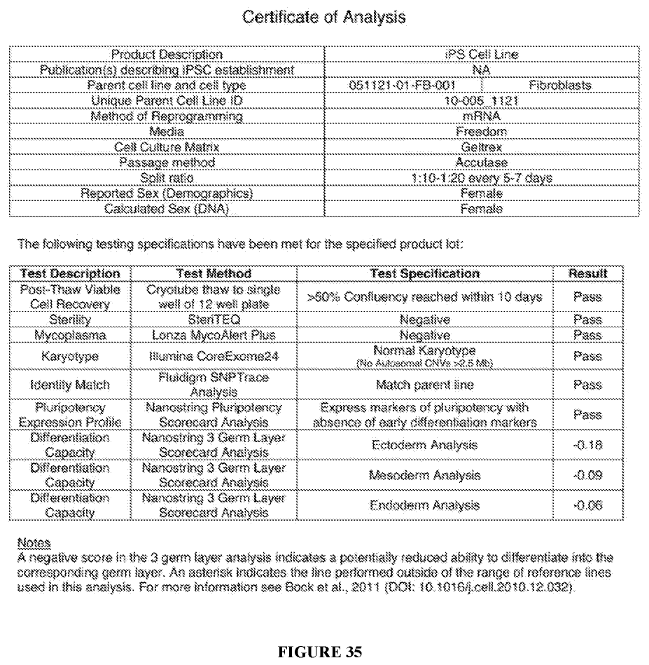

All iPSC lines were derived from skin biopsies of healthy donors. The participants were enrolled in a study approved by the Western Institutional Review Board (WIRB). This IRB-approved protocol includes the collection of biological samples, research use of these samples, and biobanking of samples. A broad consent form is utilized. iPSC lines 050743-01-MR-023 (51 y.o. male; line 1), 051106-01-MR-046 (57 y.o. female; line 2), 051121-01-MR-017 (52 y.o. female; line 3), 051104-01-MR-040 (56 y.o. female), 050659-01-MR-013 (65 y.o. female) were reprogrammed using the NYSCF Global Stem Cell Array® with the mRNA/miRNA method (StemGent), where line-to-line variability has been minimized due to the fully automated reprogramming process. iPSC lines were cultured and expanded onto Matrigel™-coated dishes in mTeSR1 medium (StemCell Technologies) or StemFlex™ medium (ThermoFisher). Lines were passaged every 3-4 days using enzymatic detachment with Accutase™ (ThermoFisher; A1110501) for 5 minutes and re-plated in mTeSR1™ medium with 10 μM ROCK Inhibitor (Y27632, Stemgent) for 24 hours. All five lines were used for CD49f+ astrocyte isolation and lines 1, 2, and 3 were then used for subsequent functional studies. All iPSC lines made though the NYSCF Global Stem Cell Array® undergo a rigorous quality check including a sterility check, mycoplasma testing, karyotyping, and a pluripotency check. A certificate of analysis (CoA) is provided upon delivery of the first cryovial. A representative CoA (from line 051121-01-MR-017) is shown in FIG. 35.

Human Brain Samples

For healthy brain immunohistochemistry, a fresh sample from the subventricular zone of a 94-year-old male brain was obtained from Advanced Tissue Services. For Alzheimer's disease brain immunohistochemistry, a fresh frozen sample of prefrontal cortex from an 80-year-old male patient diagnosed with Alzheimer's disease (Braak score VI/VI) was obtained from Rhode Island Hospital's Brain Tissue Resource Center (Title 45 CRF Part 46.102(f)). For sorting from fetal brain tissue, de-identified fetal cortical tissues from gestational week 18 (no abnormalities) were obtained under approval from the Albert Einstein College of Medicine Institutional Review Board (IRB; Study protocol 2019-10439). Due to the nature of the tissue collection procedure, the precise location of the brain where tissue originated was not able to be determined.

Animals

All animal procedures were conducted in accordance with guidelines from the National Institute of Health and Stanford University's Administrative Panel on Laboratory Animal Care (#10726) and NYU School of Medicine's Institutional Animal Care and Use Committee (#IA18-00249). All rodents were housed with food and water available ad libitum in a 12-h light/dark environment. For experiments using mice, adult female Aldh1/1eGFP transgenic mice (postnatal day, P30) on a C57BL/6J background (GENSAT, MMRRC 036071-UCD) were used. For experiments using rats, Sprague Dawley dams with 4-6-day old postnatal pups (P4-6) were purchased from Charles River (Strain code: 400). All astrocyte purification was completed in rat pups before P7.

Human Healthy Brain Immunohistochemistry

Chunks were drop fixed in 4% paraformaldehyde overnight at 4° C., washed 3× in PBS, then stored in 30% sucrose in PBS at 4° C. overnight. These were then embedded in OCT (Fisher Scientific; 50-363-579), cryosectioned at 20 μm thickness and stained using the immunofluorescence protocol described below. Tissue was incubated at 40° C. for 10 minutes and blocked with PBS containing 0.1% saponin and 2.5% donkey serum for 1 hour. Primary antibodies (see Table 11) were applied overnight at 4° C. The next day, slides were washed 3× in PBS, incubated with secondary antibodies (Alexa Fluor) and HOECHST for 1 hour at room temperature, washed 3× for 10 minutes in PBS. Secondary antibodies were used at 1:500 dilution (all Alexa Fluor from ThermoFisher). Slides were mounted and imaged on a Zeiss Confocal Microscope.

Human AD Brain Immunohistochemistry

Tissue was drop fixed in 4% PFA (overnight, 4° C.), incubated with 30% sucrose (24 hrs, 4° C.), embedded in OCT, and cryosectioned onto slides (20 μm thickness). Tissue was incubated at 40° C. for 10 minutes, blocked with 10% normal goat serum, 0.1% Tween-20 for 1 hour, then stained for CD49f (Biolegend 313602, 1:1000) and GFAP (Sigma G3893, 1:1000), and DAPI (overnight, 4 C). After secondary antibodies were applied (Abcam ab150160, 1:5000; Abcam ab150113, 1:5000), TrueBlack™ (Biotium 23007) staining was conducted according to manufacturer's protocol. Slides were imaged on a Keyence BZ-X fluorescence microscope with a 60× oil-emersion objective. Images were taken at z-stack, then full-focus merged by channel in FIJI® software. Secondary-only controls were performed, showing no observable non-specific staining.

Primary Rat Astrocyte Purification, Culture, and Staining

Astrocytes were purified by immunopanning and cultured in serum-free conditions. Briefly cortices from 5-6 postnatal day 4-6 Sprague Dawley rat pups (Charles River) were dissected out and meninges and choroid plexus removed. The cortices were minced with a scalpel and digested in Papain for 40 minutes at 34° C. under constant CO₂/O₂ gas equilibration. The digested brain pieces were washed with CO₂/O₂-equibiriated ovomucoid inhibitor solution, triturated, and spun down through a cushion gradient containing low and high ovomucoid inhibitor layers. The resulting cell pellet was passed through a 20 μm nylon mesh to create a single cell suspension. The cells were then incubated in a 34° C. water bath for 30-45 minutes to allow cell-specific antigens to return to the cell surface. Negative selection was performed using Goat anti-mouse IgG+IgM (H+L), Griffonia (Bandeiraea) simplicifolia lectin 1 (BSL-1), Rat anti-mouse CD45, and O4 hybridoma supernatant mouse IgM, followed by positive selection for astrocytes using mouse anti-human integrin β5 (ITGB5). Purified astrocytes were detached from the panning plate with trypsin at 37° C. for 3-4 min, neutralized by 30% fetal calf serum, counted, pelleted, and resuspended in 0.02% BSA in DPBS. All isolation and immunopanning steps occurred at room temperature, except for the heated digestion, incubation, and trypsinization steps. Cells were plated at 70,000 cells per well in 6 well plates containing 2 mL/well of serum-free Astrocyte Growth Medium (50% Neurobasal Medium, 50% Dulbecco's Modified Eagle Medium (DMEM), 100 U/mL Penicillin & 100 μg/mL Streptomycin, 1 mM sodium pyruvate, 292 μg/mL L-glutamine, 5 μg/mL N-acetyl-L-cysteine (NAC), 100 μg/mL BSA, 100 μg/ml Transferrin, 16 μg/mL putrescine dihydrochloride, 60 ng/mL (0.2 μM) progesterone, and 40 ng/mL sodium selenite. Immediately before plating, the astrocyte trophic factor Heparin-binding EGF-like growth factor (HBEGF) was added (5 ng/mL) and media equilibrated to 37° C. in a 10% CO₂ incubator). Cells were incubated at 37° C. in 10% CO₂ and grown for 1 week. Cells were washed with room-temperature DPBS 3×, fixed with ice-cold methanol for 20 minutes, and washed 3× with DPBS. Cells were incubated for one hour in blocking solution (PBS containing 5% donkey serum). Primary GFAP antibody (Dako; Z0334) was applied overnight at 4° C. at 1:1000. The next day, cells were washed 3× in PBS, incubated with secondary antibodies (Alexa Fluor) and HOECHST for 1 hour at room temperature, and washed 3× for 10 minutes in PBS. Secondary antibodies were used at 1:500 dilution (all Alexa Fluor from ThermoFisher). Fluorescent imaging was performed on the Opera Phenix High-Content Screening System™ (PerkinElmer) using Harmony™ analysis software.

Primary Mouse Astrocyte Sort

Using Aldh1/1^{eGFP} transgenic mice on a C57BL/6J background (GENSAT™, MMRRC 036071-UCD), a single cell suspension from the brain was created, with modifications. Briefly, following CO₂ euthanasia, brains of four adult female (P30) mice were dissected out and then the hindbrain was removed. The remaining brains were minced with a scalpel and enzymatically digested in a CO₂-equilibrated papain solution for 40 minutes in a 34° C. water bath, one brain per tube in a sealed glass bottle. The digested brain pieces were washed with CO₂-equilibrated ice cold ovomucoid inhibitor solution, triturated, and spun down through a cushion gradient containing low and high ovomucoid inhibitor layers. The resulting cell pellet was passed through a 20 μm nylon mesh to create a single cell suspension. The cells from each brain were then pooled, split into 3 conditions, and stained for 30 minutes on ice with either CD49f antibody (BD 555736), its isotype control (BD 555844), or mock stained with the working buffer. Resulting cells were then washed three times and underwent fluorescence-activated cell sorting on a Sony™ SH800Z. Red blood cells, doublets, debris, and DAPI⁺ events were excluded, and gates were drawn around the Aldh1/1⁺, Aldh1/1⁻CD49f⁺, and Aldh1/1⁻CD49f⁻ populations and were sorted into either 100 μl working buffer and imaged or directly into 350 μl Buffer RLT™ (Qiagen). The cells were imaged on a Keyence BZ-X fluorescence microscope. RNA was extracted from the cells sorted into Buffer RLT™ using the RNeasy™ kit (Qiagen). In order to determine the identity of the Aldh1/1⁻ populations, reverse transcription PCR (RT-PCR) was performed using previously verified primers targeting Aldh1/1, Gfap, Snap25, Mog, Tmem119, and Cd31 (PECAM-1). Samples containing lysed unsorted brain cells and sorted red blood cells (RBCs) were also run, along with negative controls.

Differentiation of hiPSCs into Astrocyte

Cells were cultured in a 37° C. incubator, at 5% CO₂. hiPSCs were induced along the neural lineage and differentiated as described herein. hiPSCs were plated at 1-2×10⁵ cells per well on a Matrigel™-coated six-well plate in hPSC maintenance media with 10 μM Y27632 (Stemgent; O4-0012) for 24 hours. Cells were then fed daily with hPSC maintenance media. Once colonies were ˜100-250 μm in diameter (day 0), differentiation was induced by adding neural induction medium (see Table 5 below). Cells were fed daily until day 8. On day 8, medium was switched to N2 medium (see Table 7 below) and cells were fed daily until day 12. On day 12, cells were mechanically dissociated using the StemPro™ EZPassage™ Disposable Stem Cell Passaging Tool (ThermoFisher; 23181010). Cells from each well were split into two wells of an ultra-low attachment 6-well plate and plated in N2B27 medium (see Table 8 below). From day 12 onwards, two-third media changes were performed every other day. On day 20, cells were switched to PDGF medium using a two-third media change (see Table 9 below). On the same day, aggregates that are round, with a diameter between 300 and 800 μm, and with a brown center were picked. Picked spheres were plated (20 spheres per well of a 6-well plate) onto Nunclon-Δ plates coated with 0.1 mg mL⁻¹ poly-L-ornithine (Sigma) followed by 10 μg mL⁻¹ laminin (PO/Lam coating, ThermoFisher; 23017015). Spheres were allowed to attach for 24 hours and were gently fed with PDGF medium every other day (2/3 media change). At day 60-80, spheres and the cells migrating out of the spheres were dissociated with Accutase™ (ThermoFisher; A1110501) for 30 minutes and passed through a 70 μm strainer. The resulting single-cell suspension was sorted for CD49f-positive cells. After the sort, cells were frozen in Synth-a-Freeze (ThermoFisher; A1254201) or plated onto PO/Lam coated 96-well plates for functional analyses. 24 hours after plating, medium was switched to glial medium (see Table 10 below) and cells were fed with two-third media changes every other day. Spheres remaining on the strainer at the time of the sort were plated back onto a PO/Lam Nunclon-A plates for up to three times (named first, second and third round) to maximize astrocyte yield.

Tables 5-10 provide a list of media used in the protocol.

TABLE 5
Detailed Composition of Neural Induction Medium
Neural induction medium (d0-d7)

| mTesr Custom | | | StemCell Technologies; |
|---|---|---|---|
| PenStep (100x) | 1x | | Life Technologies; 15070063 |
| SB431542 | 10 | mM | Stemgent; 04-0010 |
| LDN193189 | 250 | nM | Stemgent; 04-0074 |
| Retinoic acid | 100 | nM | Sigma-Aldrich; R2625 |

TABLE 6
Detailed Composition of Basal Medium
Basal medium

| | | |
|---|---|---|
| DMEM/F12, GlutaMAX | | ThermoFisher; 10565018 |
| PenStrep (100x) | 1x | Life Technologies; 15070063 |
| 2-Mercaptoethanol (1000x) | 1x | Life Technologies; 21985023 |
| MEM non-essential amino acids | 1x | Life Technologies; 11140-050 |
| (NEAA) solution (100x) | | |

TABLE 7
Detailed Composition of N2 Medium
N2 medium (d8-11)

| | | |
|---|---|---|
| Basal medium | | |
| N2 supplement (100x) | 1x | Life Technologies; 15070063 |
| Retinoic acid | 100 nM | Sigma-Aldrich; R2625 |
| Smoothened agonist (SAG) | 1 μM | EMD Millipore; 566660 |

TABLE 8
Detailed Composition of N2B27 Medium
N2B27 medium (d12-19)

| Basal medium | | | |
|---|---|---|---|
| N2 supplement (100x) | 1x | | Life Technologies; 15070063 |
| B27 Supplement without | 1x | | Life Technologies; 12587-010 |
| VitA (50x) | | | |
| Insulin solution, human | 25 | μg/mL | Sigma-Aldrich; 19278 |
| Retinoic acid | 100 | nM | Sigma-Aldrich; R2625 |
| Smoothened agonist (SAG) | 1 | μM | EMD Millipore; 566660 |

TABLE 9
Detailed Composition of PDGF Medium
PDGF medium (d20-sort)

| Basal medium | | | |
|---|---|---|---|
| N2 supplement (100x) | 1x | | Life Technologies; 15070063 |
| B27 Supplement without | 1x | | Life Technologies; 12587-010 |
| VitA (50x) | | | |
| Insulin solution, human | 25 | μg/mL | Sigma-Aldrich; 19278 |
| PDGFaa | 10 | ng/mL | R&D Systems; 221-AA-050 |
| IGF-1 | 10 | ng/mL | R&D Systems; 291-G1-200 |
| HGF | 5 | ng/mL | R&D Systems; 294-HG-025 |
| NT3 | 10 | ng/mL | EMD Millipore; GF031 |
| T3 | 60 | ng/mL | Sigma-Aldrich; T2877 |
| Biotin | 100 | ng/mL | Sigma-Aldrich; 4639 |
| cAMP | 1 | μM | Sigma-Aldrich; D0260 |

TABLE 10
Detailed Composition of Glial Medium
Glial medium (post-sort)

| Basal medium | | | |
|---|---|---|---|
| N2 supplement (100x) | 1x | | Life Technologies; 15070063 |
| B27 Supplement without | 1x | | Life Technologies; 12587-010 |
| VitA (50x) | | | |
| Insulin solution, human | 25 | μg/mL | Sigma-Aldrich; 19278 |
| T3 | 60 | ng/mL | Sigma-Aldrich; T2877 |
| Biotin | 100 | ng/mL | Sigma-Aldrich; 4639 |
| cAMP | 1 | μM | Sigma-Aldrich; D0260 |
| HEPES | 10 | mM | Sigma-Aldrich; H4034 |
| Ascorbic acid | 20 | μg/mL | Sigma-Aldrich; A4403 |

FACS for CD49f⁺ Astrocyte Isolation

Cells were lifted by incubation with Accutase™ for 30 minutes. Cell suspension was triturated 8-10 times and passed through a 70 μm cell strainer (Sigma; CLS431751) then diluted >7× with DMEM/F12 medium. Cells were spun in a 15 mL conical tube at 300 g for 5 minutes at room temperature. The cell pellet was resuspended in 200 μL of FACS buffer (PBS, 0.5% BSA, 2 mM EDTA, 20 mM Glucose) with 1:50 PE Rat Anti-Human CD49f antibody (BD Biosciences; 555736) and incubated on ice for 20 minutes. Cells were then washed in FACS buffer, pelleted at 300 g for 5 minutes and resuspended in FACS buffer containing propidium iodide for dead cell exclusion. The respective unstained, CD49f-only stained, and propidium iodide-only stained controls were run in parallel. CD49f⁺ cells were isolated via FACS on an ARIA-IIu™ Cell Sorter (BD Biosciences) using the 100 μm ceramic nozzle, at 20 or 23 psi. Data were analyzed using FlowJo™ v9.

For initial screening, BD Lyoplate-Human cell surface marker screening panel (BD Biosciences: 560747) and O4 antibody were used on day 78 cultures digested as described above.

For A1 stimulation, cells were treated with TNFα (30 ng/mL), IL-1α (3 ng/mL), and C1q (400 ng/mL) for 24-48 hours.

Differentiation into Oligocortical Organoids and Single-Cell Digestion

hiPSC line 051121-01-MR-017 was induced along the neural lineage and differentiated into oligocortical organoids. At days 117 and 169, organoids were digested into a single-cell suspension for FACS sorting. For digestion, 4 organoids were pooled and incubated in papain (Worthington: LK003150) for 30 minutes at 37° C. on a shaker. The cell suspension was triturated 10 times and placed back at 37° C. for 10 more minutes. The ovomucoid protease inhibitor was added and the cell suspension was spun down at 300 g for 4 minutes, resuspended in FACS buffer, and filtered through a 45 μm filter. The cell suspension was then stained for PE Rat Anti-Human CD49f antibody with appropriate controls and CD49f positive and negative fractions were isolated as described above. CD49f⁺ and CD49f⁻ cell fractions were plated down on poly-ornithine and laminin-coated plates and fixed in 4% PFA for immunofluorescence analysis.

Oligocortical organoids were also fixed in 4% paraformaldehyde 1 hour at R.T., washed 3× in PBS, then stored in 30% sucrose in PBS at 4° C. overnight. Organoids were then embedded in O.C.T. compound and cryosectioned at 20 μm thickness.

Immunofluorescence

Cells were fixed in 4% paraformaldehyde for 10 minutes, washed 3× in PBS, and incubated for one hour in blocking solution (PBS containing 0.1% Triton-X100 and 5% donkey serum). Primary antibodies (see Table 11) were applied overnight at 4° C. The next day, cells were washed 3× in PBS, incubated with secondary antibodies (Alexa Fluor) and HOECHST for 1 hour at room temperature, and washed 3× for 10 minutes in PBS. Secondary antibodies were used at 1:500 dilution (all Alexa Fluor from ThermoFisher). For fluorescent image analysis and quantification of GFAP, AQP4, C3, and cell radial mean plates were imaged on the Opera Phenix High-Content Screening System™ (PerkinElmer) using Harmony™ analysis software. For MAP2 quantification plates were imaged on the ArrayScan™ XTI live high content platform (Thermo Fisher) and used CellProfiler™ software.

Antibodies used in these studies are shown in Table 11.

TABLE 11
Antibodies For Immunofluorescence Studies

| | | | Cat. No. |
|---|---|---|---|
| Name | Dilution | Vendor | (RRID) |
| GFAP | 1:1000 | EMD Millipore | MAB360 |
| | | | (AB_11212597) |
| S100B | 1:1000 | Sigma | S2532 |
| | | | (AB_477499) |
| Vimentin | 1:250 | Abcam | ab8978 |
| | | | (AB_306907) |
| C3D | 1:1000 | DAKO | A0063 |
| | | | (AB_578478) |
| NFIa | 1:250 | Active Motif | 39397 |
| | | | (AB_2314931) |
| AQP4 | 1:500 | Sigma | HPA014784 |
| | | | (AB_1844967) |
| CD49f | 1:1000 | BioLegend | 313602 |
| | | | (AB_345296) |
| SOX9 | 1:250 | Cell Signaling | 82630T |
| | | | (AB_2665492) |
| EAAT1 | 1:250 | Abcam | ab416 |
| | | | (AB_304334) |
| MAP2 | 1:1000 | Abcam | ab5392 |
| | | | (AB_2138153) |
| O4 | 1:50 | Gift from Dr. J. Goldman | |
| CD49f | 1:50 (FACS) | BD Biosciences | 555736 |
| | | | (AB_396079) |

Immunofluorescence on Slides

Slides with cryosectioned organoids were incubated for one hour in blocking solution (PBS containing 0.1% saponin and 2.5% donkey serum). Primary antibodies (see Table 11) were applied overnight at 4° C. The next day, slides were washed 3× in PBS, incubated with secondary antibodies (Alexa Fluor) and HOECHST for 1 hour at room temperature, washed 3× for 10 minutes in PBS. Secondary antibodies were used at 1:500 dilution (all Alexa Fluor from ThermoFisher). Slides were mounted and imaged on a Zeiss Confocal Microscope.

Glutamate Uptake Assay

CD49 f⁺ astrocytes were incubated for 30 minutes in Hank's balanced salt solution (HBSS) buffer without calcium and magnesium (Gibco), then for 3 hours in HBSS with calcium and magnesium (Gibco) containing 100 μM glutamate. At the same time, identical volumes of HBSS with calcium and magnesium (Gibco) containing 100 μM glutamate were incubated in empty cell-free wells for determining the percentage of glutamate uptake. Samples of medium were collected after 3h and analyzed with a colorimetric glutamate assay kit (Sigma-Aldrich; MAK004-1KT), according to the manufacturer's instructions. Samples of HBSS with calcium and magnesium (Gibco) without glutamate were also run as negative controls. For A1 astrocytes, cells were treated with 3 ng/mL IL-1α (Sigma; I3901), 30 ng/mL tumor necrosis factor alpha (R&D Systems; 210-TA-020) and 400 ng/mL C1q (MyBioSource; MBS143105) for 24h prior to the experiment. Three iPSC lines and two to four technical replicates per lines were used. p-values were calculated using a one-way ANOVA with Dunnett's correction for multiple comparisons for comparing astrocytic glutamate uptake to the no astrocyte control or using multiple t-tests with Holm-Sidak's correction for multiple comparisons for comparing A1 astrocytes to A0 astrocytes.

Intracellular Ca²⁺ Imaging on hiPSC-Astrocytes

CD49f⁺ astrocytes from three iPSC lines were cultured on glass coverslips coated with 0.1 mg/ml poly-L-ornithine followed by 10 μg/ml laminin. For Ca²⁺ dye loading, cells were treated with Rhod-3/AM (ThermoFisher; R10145) for 30 minutes at 37° C., washed twice with glial medium and imaged 30-60 minutes later. Live fluorescence imaging of spontaneous Ca²⁺ activity was done with an ArrayScan™ XTi high-content imager (ThermoFisher) equipped with live cell module maintaining 37° C., 5% CO₂ and >90% relative humidity environment. Whole field of view images at 20× magnification were acquired with Photometrics™ X1 cooled CCD camera (ThermoFisher) at 4 Hz for 2 minutes. For Ca²⁺ imaging experiments involving drug application cells were grown on 1.5× PO/Lam coated plastic coverslips (Nunc Thermanox) and then transferred to a heated (31° C.) recording chamber mounted onto an upright Olympus™ BX61 microscope. Fluorescence was recorded at 2 Hz by a cooled CCD camera (Hamamatsu™ Orca R²). Images were taken 2 minutes before and 3 minutes after the addition of ATP (100 μM), and drug application was done via whole chamber perfusion for a period of 60s. For quantification of the change in intensity over time, astrocytes were outlined as regions of interest (ROIs) and analyzed with ImageJ™ software. [Ca²⁺]ᵢ transients are expressed in the form of ΔF(t)/F₀, where F₀ is a baseline fluorescence of a given region of interest and ΔF is the difference between current level of fluorescence F(t) and F₀. Fluctuations of ΔF(t)/F₀ of less than 0.05 were considered non-responses.

Intracellular Ca²⁺ Imaging on Rodent Astrocytes

Postnatal rat astrocytes were purified by immunpanning (see above), plated at a density of 5,000 cells/35 mm glass-bottom dish (MatTek, No. 1.5) coated with poly-D-lysine, and maintained in serum-free culture conditions for 5-7 days. For imaging, astrocytes were pre-incubated for 15 minutes with 2 μM Fluo-4 AM (Invitrogen, F-14201) and washed with 1×PBS and replaced with normal astrocyte growth medium. Fluorescent image stacks were taken at 0.7 s intervals and intensity analyzed for 10-30 randomly selected cells per stack in ImageJ™. Data was collected from three separate preparations of astrocyte cultures from at least three different plates of cells per preparation.

Neuronal Differentiation and Co-Culture with Astrocytes

For neuronal differentiation, hiPSCs (line 3) were plated in a 12-well plate in hPSC maintenance media with 10 μM ROCK inhibitor (Y2732, Stemgent). The next day, the cells were induced and fed daily with neural induction media (DMEM/F12 (ThermoFisher; 11320033) 1:1 Neurobasal (ThermoFisher; 21103049) with 1× Glutamax, 1× N2 supplement, 1× B27 supplement without Vitamin A) with SB431542 (20 μM), LDN193189 (100 nM), XAV939 (1 μM). On day 10, the media was switched to neural induction media with XAV939 (1 μM), and the daily media changes continued. On day 15, cells were dissociated using Accutase™ and either frozen in Synth-a-freeze, or plated in neuronal media (Brainphys™ (StemCell Technologies; 05790) with 1× B27 supplement (ThermoFisher; 17504001), and 10 μM ROCK inhibitor) at 50 k/well in a PO/Lam coated 96-well plate (Corning; 353376). On day 16, the media was switched to neuronal media with BDNF (40 ng/mL), GDNF (40 ng/mL), Laminin (1 μg/mL), dbcAMP (250 μM), ascorbic acid (200 μM), PD0325901 (10 μM), SU5402 (10 μM), DAPT (10 μM). Cells were fed every other day. PD0325901, SU5402, and DAPT were taken out of the media after two weeks. CD49f⁺ astrocytes were plated on top of neurons on day 33 of neuronal differentiation and cells were fed every other day with neuronal media until day 50.

To study the effect of A1 astrocytes on neuronal maturation, CD49f⁺ astrocytes (15,000/well of a 96 well plate) were plated on top of neurons on day 33 of neuronal differentiation and cells were fed twice per week with neuronal media with or without TNFα, IL-1α, and C1q until day 51-53. To evaluate the potential direct effect of cytokines on neuronal maturation, neurons cultured alone were fed twice per week, starting at day 34 with neuronal media with or without TNFα, IL-1α, and C1q until day 53.

Electrophysiology

For whole-cell recordings, hiPSC-derived neurons from one iPSC line (line 3) were visualized using an upright Olympus BX61 microscope equipped with a 40× objective and differential interference contrast optics. Neurons were constantly perfused with Brainphys™ medium (STEMCELL Technologies, Catalog #05790) preheated to 30-31° C. Patch electrodes were filled with internal solutions containing 130 mM K⁺gluconate, 6 mM KCl, 4 mM NaCl, 10 mM Na⁺HEPES, 0.2 mM K⁺EGTA; 0.3 mM GTP, 2 mM Mg²⁺ ATP, 0.2 mM cAMP, 10 mM D-glucose. The pH and osmolarity of the internal solution were adjusted to resemble physiological conditions (pH 7.3, 290-300 mOsmol). Current- and voltage-clamp recordings were carried out using a Multiclamp™ 700B amplifier (Molecular Devices), digitized with Digidata™ 1440A digitizer and fed to pClamp™ 10.0 software package (Molecular Devices). For spontaneous EPSC recordings, neurons were held at chloride reversal potential of −75 mV. Data processing and analysis were performed using ClampFit™ 10.0 (Molecular Devices) and Prism software. CD49f⁺ astrocytes for co-cultures were from three iPSC lines. p-values to compare neurons alone to neurons with astrocytes or neurons with A0 astrocytes to neurons with A1 astrocytes were calculated using a two-tailed, unpaired t-test.

Neurotoxicity

Primary mouse cortical neurons (ThermoFisher; A15586) were thawed and plated into PO/Lam coated 96-well plates at a density of 20 k/well in Neurobasal media (ThermoFisher; 21103049) with 1× Glutamax, 1× B27 supplement (ThermoFisher; 17504001), and 1× PenStrep (Life Technologies; 15070063). Cells were fed the next day, then every other day until the addition of astrocyte conditioned media ten days later. CD49f⁺ astrocytes were plated into PO/Lam coated 24-well plates at a density of 200 k/well and kept in PDGF medium for 24 hours. The next day, the medium was switched to Neurobasal medium with 1× Glutamax, 1× PenStrep and 1× B27 supplement, and half-media changes were performed every other day, for one week. At day 8, a full media change was performed, with 600 μL of Neurobasal medium, 1× Glutamax, 1× PenStrep and 1× B27 supplement, minus antioxidants (ThermoFisher; 10889038) per well, with or without TNFα, IL-1α, and C1q. Astrocyte conditioned media (ACM) from CD49f⁺ A0 astrocytes (unstimulated) and A1 astrocytes (cultured with TNFα, IL-1α, and C1q) were collected 48 hours later and added to the mouse neuronal cultures without concentration. Mouse neurons were treated with 70% ACM with 5 μM IncuCyte Caspase-3/7 Green Apoptosis Assay Reagent™ (Sartorius; 4440) and imaged every 6 hours for 60 hours on an Incucyte™ S3 epifluorescence time lapse microscope (Sartorius). Mouse neurons were also treated with fresh media controls, with or without TNFα, IL-1α, and C1q. Two to four wells were analyzed per condition. For image analysis, we took 3 images per well using a 10× objective lens from random areas of the 96-well plate and plotted the total integrated intensity, known as the total sum of the objects' fluorescent intensity in the image. Data was normalized to the confluence per image. Data analysis was done using the Incucyte™ analysis software (Sartorius). Graphpad Prism™ software was used to perform a two-way ANOVA to determine statistical significance per line across conditions.

Human iPSC-neurons from one iPSC line (line 3) were differentiated as previously described until the addition of astrocyte conditioned media from three iPSC lines at day 66 of the differentiation. CD49f⁺ astrocytes were plated into PO/Lam coated 24-well plates at a density of 200 k/well in PDGF medium. The next day, the medium was switched to Brainphys™ medium with 1× PenStrep and 1× B27 supplement, then half-media changes were performed every other day. At day 8, a full media change was performed, with 600 μL of Brainphys™, 1× PenStrep, and 1× B27 supplement minus antioxidants per well, with or without TNFα, IL-1α, and C1q. Astrocyte conditioned media (ACM) from CD49f A0 astrocytes (unstimulated) and A1 astrocytes (cultured with TNFα, IL-1α, and C1q) was collected 48 hours later and applied to the hiPSC-neuron cultures without any concentration. hiPSC-neurons were treated with 67% ACM with 5 μM IncuCyte™ Caspase-3/7 Green Apoptosis Assay Reagent (Sartorius; 4440) and 1:2000 IncuCyte™ NucLight Rapid Red Reagent for nuclear labeling (Sartorius; 4717) and imaged every 6 hours for 72 hours on an Incucyte™ S3 epifluorescence time lapse microscope (Sartorius). hiPSC-neurons were also treated with fresh media controls, with or without TNFα, IL-1α, and C1q. Four to eight wells were analyzed per condition. For image analysis, we took 3 images per well using a 10× objective lens from random areas of the 96-well plate and plotted the percentage of caspase-3/7 positive nuclei. Data analysis was done using the Incucyte™ analysis software (Sartorius). Graphpad Prism™ software was used to perform a two-way ANOVA to determine statistical significance per line across conditions.

Synaptosome Engulfment Assay

Live CD49f⁺ astrocytes were plated on PO/Lap coated 96 well plates and treated with TNFα, IL-1α, and C1q for 48 hours. Cells were then incubated with 2 μL/mL pHrodo-conjugated synaptosomes in glial medium with or without TNFα, IL-1α, and C1q and imaged every hour with an Incucyte™ S3 epifluorescence time lapse microscope (Sartorius) for 2 days. Three iPSC lines and three or four wells/line were analyzed per condition. For image analysis, 3 images per well were taken using a 10× objective lens from random areas of the 96-well plate and plotted the total integrated intensity, known as the total sum of the objects' fluorescent intensity in the image. Data was normalized to the confluence per image. Data analysis was done using the Incucyte™ analysis software (Sartorius). Graphpad Prism™ software was used to perform a two-way ANOVA to determine statistical significance per line across conditions.

Bulk RNA Sequencing and Analysis

RNA isolation was performed using the RNeasy™ Plus Micro Kit (Qiagen; 74034). Media was aspirated off CD49f cells in culture, and cells were lysed in Buffer RLT™ Plus with 1:100 β-mercaptoethanol. Samples were then stored at −80° C. until processed further according to manufacturer's instructions. RNA was eluted in 17 μl RNase free ddH2O and quantified with a Qubit™ 4 Fluorometer (ThermoFisher; Q33227). Paired-end RNAseq data were generated with the Illumina HiSeq™ 4000 platform following the Illumina protocol. The raw sequencing reads were aligned to human hg19 genome using star aligner (Dobin et al. 2013) (version 2.4.0 g1). Following read alignment, featureCounts™ (v1.6.3) was used to quantify the gene expression at the gene level based on Ensembl™ gene model GRCh37.70. For re-analysis of human primary astrocyte, raw RNAseq data was downloaded from gene expression omnibus (GEO: accession GSE73721). Similarly, for comparison with a recently published hiPSC-derived astrocyte datasets, three bulk RNAseq raw data was downloaded from GSE97904, GSE133489, GSE99951. The RNAseq data from each of these published studies were processed using the same star/featureCounts pipeline as described above and then the gene level read counts were combined with the gene count data of samples described herein. Genes with at least 1 count per million (CPM) in more than 2 samples in the merged data were considered expressed and hence retained for further analysis, otherwise removed. Then the read count data were normalized using trimmed mean of M-values normalization (TMM) method to adjust for sequencing library size difference and then corrected for batch using linear regression. To examine similarities among samples, hierarchical cluster analysis was performed based on the respective transcriptome-wide gene expression data using R programming language. Meanwhile, a separate expression abundance, transcript per million (TPM), was also calculated using salmon (v0.14.1) with 50 bootstraps and fragment-level GC biases correction enabled for optimizing the abundance estimation.

Single-Cell RNA Sequencing

For A1-like astrocyte analysis, day 73 (line 3) or day 80 (line 1) unsorted hiPSC-derived cultures (to include astrocytes at different stages of development) were left untreated or were treated for 24 hours with TNFα, IL-1α, and C1q, then harvested in parallel using papain (Worthington; LK003153) and processed using the 10× Single Cell™ 3′ v2 or v3.1 protocols. For CD49f sorting experiment, day 73 (line 3) unsorted cultures were harvested using papain, sorted for CD49f, and the unsorted, CD49f, and CD49f fractions were processed using the 10× Single Cell 3′ v3.1 protocol. For fetal samples, unsorted, sorted CD49f⁺, and sorted CD49f⁻ cells were processed using the 10× Single Cell™ 3′ v3.1. All samples were filtered through 40 μm Flowmi™ Cell Strainers (Scienceware; H13680-0040) to obtain a single cell suspension. In brief, the Chromium™ Single Cell A Chip (10× Genomics; PN-1000009) or the Chromium™ Next GEM Chip G Single Cell Kit (10× Genomics; PN-1000120) was loaded with ˜7,000 cells/sample and library preparation was performed as per the Chromium™ Single Cell 3′ Library & Gel Bead Kit manufacturer's recommendations (10× Genomics; PN-120237 and PN-1000121). The Chromium™ i7 Multiplex Kit (10× Genomics; PN-120262) was used. Quality control was performed using the Qubit™ 4 Fluorometer (ThermoFisher; Q33227) and the Agilent 4200 TapeStation™ system. The resulting prepared cDNA library was sequenced on a NovaSeq/HiSeg™ 2×150 bp, and ˜50,000 reads per cell were obtained.

Single-Cell Data Analysis.

Sequenced samples were initially processed using Cell Ranger™ software version 3.0.2 (10× Genomics) and were aligned to the GRCh38 (hg38) human reference genome. Through the Cell Ranger pipeline, digital gene expression matrices (DGE) were generated containing the raw unique molecular identifier (UMI) counts for each sample. In order to compare between samples, DGEs were merged using the muscat R™ package. Doublets were removed using the hybrid method of the scds package, therefore excluding the predicted 1% per thousand cells with the highest doublet scores. Quality control and filtering was completed using the scater R™ package. Cells were removed if their feature count, number of expressed features, and/or percentage of mitochondrial genes was outside of the median value±2.5 median absolute deviations. Genes were removed if they were undetected across all cells or if they were expressed by fewer than 20 cells. For hiPSC samples, of the 53,599 cells and 28,158 genes originally identified, 43,127 cells (81%) and 13,710 genes (49%) met these criteria and were included in the following analyses. For fetal samples, of the 29,585 cells and 28,161 genes originally identified, 22,281 cells (75%) and 14,146 genes (50%) met these criteria and were included in the following analyses.

Next, samples were integrated, clustered, and dimensionally reduced using Seurat™ version 3.1.0. The top 2000 variable genes, identified using FindVariableFeatures™ function, were used to integrate and cluster samples. Integration was performed using the first 30 dimensions of the Canonical Correlation Analysis (CCA) cell embeddings. Clusters and tSNE plots were generated using the first 20-30 principle components, and a resolution of 0.1-0.3 was used to cluster all cells (please see Table 12 below for specifics depending on round of analysis). Clusters were then identified manually based on a known set of neural cell-specific markers. New subset objects were made in order to analyze specific astrocyte-related clusters and/or samples (please see Table 12 below for subsetting strategy). This process was repeated separately for fetal samples.

Table 12. Principle components and resolution used for analyses. Object key: so_astro_plated, data from only astrocyte-related clusters from only A0/A1-like plated hiPSC-derived samples; so_sorted, data from only sorted hiPSC-derived samples; so_sorted_astro, data from only astrocyte-related clusters from only sorted hiPSC-derived samples; so_fetal, data from all fetal samples.

| | | Object from | | | |
|---|---|---|---|---|---|
| | | which subset | Principle | | |
| Purpose | Object name | was generated | Components | Resolution | FIG. |
| Analyze | so_astro_plated | so_astro | 20 | 0.3 | 24A-D |
| hiPSC astrocyte- | | | | | |
| Related clusters from | | | | | |
| plated samples | | | | | |
| Analyze only | so_sorted | so | 30 | 0.3 | 19A-D |
| sorted samples | | | | | 28 |
| | | | | | 29 |
| Analyze | so_sorted_astro | so_sorted | 20 | 0.2 | 19E-G |
| astrocyte-related | | | | | 30 |
| clusters from only | | | | | |
| sorted samples | | | | | |
| Analyze all | so_fetal | N/A | 30 | 0.1 | 20B-D |
| fetal samples | | | | | |

Through these analyses, in all hiPSC samples, 13 subpopulations were identified, including mature astrocytes, transitioning astrocytes, immature astrocytes, neural progenitor cells, oligodendrocytes, and neurons. In fetal samples, 18 subpopulations were identified, including mature astrocytes, immature astrocytes, oligodendrocyte precursor cells, myeloid cells, endothelial cells, and cells of unknown origin. tSNE plots, feature plots, and dot plots were generated using Seurat standard functions. Additionally, differential gene expression tables (data not shown) were generated using the FindAllMarkers™ function. Additionally, differential gene expression tables (data not shown) were generated using the FindAllMarkers™ function.

qRT-PCR

Reverse transcription was performed using the iScript™ cDNA Synthesis Kit (Biorad; 1708891) with 500 ng of RNA per reaction. Real-Time PCR was then performed on an Applied Biosystems™ 7300 Real-time PCR system with 5 ng cDNA per sample in triplicate using Taqman™ gene expression master mix (ThermoFisher; 4369514) and the following pre-designed Taqman™ gene expression assays (ThermoFisher; 4331182): ITGA6 (Hs01041011_m1), MEGF10 (Hs01002798_m1), MERTK (Hs00179024_m1), ITGA6 (Hs01090305_m1), GRIN2b (Hs01002012_m1), GRIA1 (Hs00181348_m1), GRIK1 (Hs00543710_m1), THBS1 (Hs00962908_m1), THBS2 (Hs01568063_m1), SPARCL1 (Hs00949886_m1), GPC6 (Hs00170677_m1), and ACTB (Hs01060665_g1). StepOnePlus™ Software (ThermoFisher) was used to determine Ct values. Expression values were normalized to ACTB and to A0 samples. CD49f⁺ astrocytes from three lines were untreated or treated with TNFα, IL-1α, and C1q for 24 hours before mRNA was collected. Two independent experiments were performed per line. Three technical replicates were run per sample. Graphpad Prism software was used to perform a paired t-test analysis to determine statistical significance across conditions.

Fetal Brain Digestion for Single Cell Suspension

Tissues were chopped and incubated in papain (Worthington; LK003150) for 30 minutes at 37° C. on a shaker. The cell suspension was triturated 10 times and placed back at 37° C. for 10 more minutes. The ovomucoid protease inhibitor was added and the cell suspension was spun down at 300 g for 4 minutes, resuspended in distilled water for 30 seconds for red blood cell lysis, diluted in FACS buffer, spun down at 300 g for 4 minutes, resuspended in FACS buffer, and filtered through a 45 μm filter. The cell suspension was then stained for PE Rat Anti-Human CD49f antibody with appropriate controls and CD49f positive and negative fractions were FACS-isolated as described above, except using a 130 μm nozzle, which is recommended for larger and adherent cells to reduce the likelihood of clogging; however, it may reduce the purity of the sorted populations. Unsorted, CD49f⁺, and CD49f⁻ cell fractions were processed using the 10× Single Cell 3′ v3.1 protocol as described above or plated down on poly-ornithine and laminin-coated plates, fixed in 4% PFA in PBS 3 days later for 10 minutes, then washed 3× and stored in PBS.

Protein Quantification

Unstimulated astrocytes (A0) or astrocytes stimulated for 24 hours with TNFα, IL-1α, and C1q were lysed with protein lysis buffer consisting of RIPA™ buffer (Sigma; R0278), cOmplete™ Mini EDTA-free Protease Inhibitor 534 Cocktail (Sigma; 11836170001), Phosphatase Inhibitor Cocktail 3™ (Sigma; P0044), and Phosphatase 535 Inhibitor Cocktail 2™ (Sigma; P5726). Protein concentration for lysate samples was determined using a Pierce BCA Protein Assay Kit (Thermo Scientific; 23225). To quantify protein levels, equal amounts of protein per sample were run on the Wes™ (ProteinSimple). The following primary antibodies were used at a 1 to 50 dilution: ITGA6 antibody (Novus Biologicals; NBP1-85747), EAAT2 antibody (Santa Cruz Biotechnology; sc-365634), GFAP antibody (Dako; Z0334), TIMP-1 antibody (R&D Systems; AF970), beta-actin antibody (Santa Cruz Biotechnology; sc-47778). ProteinSimple™ Detection Modules were used for as secondary antibodies. Protein was collected from three cell lines and two independent experiments. Two technical replicates were run per sample. Protein levels were determined using Compass™ software (ProteinSimple™) as the area under the curve and were normalized to beta-actin. Graphpad Prism™ software was used to perform a paired t-test to determine statistical significance between conditions.

Quantification and Statistical Analysis

The software used for quantification is specified for each assay. Briefly, Graphpad Prism™ software was used for all statistical analyses. The statistical test used, value of n, and meaning of n are indicated in the figure legends and the corresponding methods section. The definition of center and dispersion and precision measures are indicated in the figure legends. Statistical significance is defined as p<0.05 (*=p<0.05; **=p<0.01, ***=p<0.001, ****=p<0.0001).

Additional Resources

RNA sequencing datasets from this study are available in a user-friendly searchable online database (nyscfseq.appspot.com).

Although the objects of the disclosure have been described with reference to the above example, it will be understood that modifications and variations are encompassed within the spirit and scope of the disclosure. Accordingly, the disclosure is limited only by the following claims.

What is claimed is:

1. A method for isolating an astrocyte from a mixed population of cells comprising:
a) selecting for a CD49f+ cell from the mixed population; and
b) sorting and isolating the CD49f+ cell from the mixed population, wherein the CD49f+ cell is a CD49f+ astrocyte, thereby isolating the astrocyte.

2. The method of claim 1, wherein the mixed population of cells comprises neuronal cells derived from stem cells (SCs).

3. The method of claim 2, wherein the SCs are induced pluripotent stem cells (iPSCs).

4. The method of claim 3, wherein the iPSCs are generated by reprogramming a somatic cell.

5. The method of claim 1, wherein the astrocyte is mammalian.

6. The method of claim 5, wherein the astrocyte is human.

7. The method of claim 2, wherein the mixed population of cells has been cultured for less than about 120, 110, 100, 90, 80, 70 or 60 days.

8. The method of claim 1, wherein the mixed population of cells is an organoid.

9. The method of claim 1, wherein selecting comprises contacting the mixed population of cells with an antibody that selectively binds CD49f.

10. The method of claim 9, wherein the antibody is fluorescently labeled and/or bound to a bead, the bead optionally being magnetic.

11. The method of claim 10, wherein the astrocyte is sorted by flow cytometry.

12. The method of claim 11, wherein the astrocyte is sorted by fluorescence-activated cell sorting (FACS).

13. The method of claim 1, wherein the CD49f+ astrocyte expresses one or more genes selected from CHI3L1, ALDH1L1, TLR4, ALDOC, FBXO21, NUDT3, TMX2, CPE, GLUL, HISTIH2AI, HISTIH3E, TPX2, NUSAP1, PPDPF or any combination thereof.

14. A method of generating and isolating an astrocyte comprising:
a) generating a mixed population of cells by culturing a stem cell (SC) under conditions to induce neuronal differentiation;
b) selecting for a CD49f+ cell from the mixed population of cells; and
c) isolating the CD49f+ cell from the mixed population of cells, wherein the CD49f+ is a CD49f+ astrocyte, thereby generating and isolating the astrocyte.

15. The method of claim 14, wherein a) comprises:
i) culturing the SC in a neural induction medium;
ii) culturing the cells of a) in a culture medium comprising retinoic acid and smoothened agonist (SAG); and
iii) culturing the cells of ii) in a culture medium comprising platelet-derived growth factor (PDGF), hepatocyte growth factor (HGF), insulin-like growth factor 1 (IGF-1), and neurotrophin 3 (NT3).

16. The method of claim 15, further comprising culturing the SC in a medium containing an inhibitor of rho-associated protein kinase (ROCK) prior to i).

17. The method of claim 16, wherein the inhibitor of ROCK is Y27632.

18. The method of claim 15, wherein the neural induction medium is as set forth in Table 1.

19. The method of claim 18, wherein the SC is cultured in the neural induction medium for about 7 to 8 days.

20. The method of claim 15, wherein the medium of ii) is as set forth in Tables 6 and 7.

21. The method of claim 20, wherein the cells of ii) are cultured for about 3, 4 or 5 days.

22. The method of claim 15, wherein the cells of i), ii) and iii) are cultured on an adherent matrix.

23. The method of claim 15, wherein the medium of iii) is as set forth in Table 5.

24. The method of claim 15, wherein the cells of iii) are cultured for about 40 to 60 days.

25. The method of claim 15, wherein the astrocyte is isolated after a total culture duration of about 60 to 80 days.

26. The method of claim 25, further comprising expanding the isolated CD49f+ astrocyte by culturing the cell in a cell expansion medium.

27. The method of claim 26, wherein the cell expansion medium is as set forth in Table 6.

28. The method of claim 14, wherein the isolated CD49f+ astrocyte is co-cultured with a neuronal cell.

29. The method of claim 14, wherein the SC is an induced pluripotent stem cell (iPSC).

30. The method of claim 29, wherein the iPSC is generated by reprogramming a somatic cell.

31. The method of claim 14, wherein the astrocyte is mammalian.

32. The method of claim 31, wherein the astrocyte is human.

33. The method of claim 14, selecting comprises contacting the mixed population of cells with an antibody that selectively binds CD49f.

34. The method of claim 33, wherein the antibody is fluorescently labeled.

35. The method of claim 34, wherein the astrocyte is sorted by flow cytometry.

36. The method of claim 35, wherein the astrocyte is sorted by fluorescence-activated cell sorting (FACS).

37. A kit comprising:
a) an antibody that selectively binds CD49f; and
b) a reagent for generating, culturing and/or isolating a CD49f+ astrocyte.

38. The method of claim 36, wherein the antibody is optionally labeled and is optionally bound to a bead, and wherein the bead is optionally magnetic.

39. The method of claim 37, wherein the antibody is fluorescently labeled.

40. The kit of claim 37, wherein the reagent comprises a buffer for performing fluorescence-activated cell sorting (FACS).

41. The kit of claim 38, wherein the reagent comprises a component of a cell culture medium.

42. The kit of claim 42, wherein the cell culture medium is a culture medium as set forth in any one of Tables 5-11 or any combination thereof.

43. The kit of claim 37, wherein the reagent comprises an inhibitor of transforming growth factor beta (TGFβ) signaling, an inhibitor of bone morphogenetic protein (BMP) signaling, an inhibitor of rho-associated protein kinase (ROCK), or any combination thereof.

44. The kit of claim 37, wherein the reagent comprises retinoic acid (RA), smoothened agonist (SAG), or a combination thereof.

45. The kit of claim 44, wherein the reagent further comprises SB431542, LDN193189, or a combination thereof.

46. A method comprising culturing the isolated CD49f+ astrocyte of any of claims 1-36 with a neuronal cell.

47. The method of claim 46, wherein an organoid is generated.

48. A method of treating a neurological disease or disorder in a subject comprising administering to the subject an effective amount of the isolated CD49f+ astrocyte of any of claims 1-36, thereby treating the neurological disease or disorder in the subject.

49. The method of claim 48, wherein the neurological disease or disorder is a neurodegenerative disease.

50. The method of claim 48, wherein the SC is an iPSC generated from a somatic cell of the subject.

51. The method of claim 50, wherein the subject is human.

52. A non-human mammal comprising the isolated astrocyte of any of claims 1-36.
